(19) United States
(12) Patent Application Publication
(10) Pub. No.: US 2020/0231562 A1
(43) Pub. Date: Jul. 23, 2020

(54) NOVEL MACROLIDE ANTIBIOTICS

(71) Applicants: HELMHOLTZ-ZENTRUM FÜR INFEKTIONSFORSCHUNG GMBH, Braunschweig (DE); Leibniz-Institut Für Naturstoff-Forschung Und Infektionsbiologie E.V. (HKI), Jena (DE); GOTTFRIED WILHELM LEIBNIZ UNIVERSITAT HANNOVER, Hannover (DE)

(72) Inventors: Frank Surup, Manden (DE); Heinrich Steinmetz, Hildesheim (DE); Kathrin Mohr, Braunschweig (DE); Konrad Viehrig, Saarbrucken (DE); Rolf Müller, Blieskastel (DE); Markus Nett, Jena (DE); Sebastian Schieferdecker, Jena (DE); Hans-Martin Dahse, Jena (DE); Michael Wolling, Hannover (DE); Andreas Kirschning, Celle (DE)

(21) Appl. No.: 15/324,608

(22) PCT Filed: Jul. 7, 2015
(86) PCT No.: PCT/EP2015/001384

§ 371 Date: Jan. 6, 2017

(30) Foreign Application Priority Data
Jul. 7, 2014 (EP) 14002319.3

(51) Int. Cl. C07D 313/00; C07H 17/08; A61P 31/04

(52) U.S. Cl. CPC C07D 313/00; A61K 45/06; A61P 31/04; C07H 17/08

(57) ABSTRACT

The present invention relates to a compound according to general formula (I), which exhibits bioactivity; methods for the production of the compound; to pharmaceutical compositions comprising one or more of the compound(s); and to the use of the compound(s) as a medicament.

### FIELD OF THE INVENTION

The present invention relates to a compound according to general formula (I), which exhibits bioactivity; methods for the production of the compound; to pharmaceutical compositions comprising one or more of the compound(s); and to the use of the compound(s) as a medicament, e.g. an antibiotic.

### BACKGROUND OF THE INVENTION

Myxobacteria are a prolific source of novel secondary metabolites, e. g. the anticancer drug epothilone, the antibacterial sorangicin and the antifungal soraphen. In recent years, full genome sequencing of various strains led to the identification of novel secondary metabolites with antibacterial, antifungal or anticancer activity.

In view of the rapid decline in the effectiveness of antibiotics due to the emergence of resistance, there is a need for a constant supply of new antibiotics for effective treatment of bacterial infections. For instance, the steadily rising occurrence of methicillin-resistant Staphylococcus aureus (MRSA) and vancomycin-resistant Staphylococcus aureus (VRSA) strains poses a serious public health threat, since Staphylococcus aureus (S. aureus) inter alia causes severe infections of the skin, respiratory disease, and food poisoning.

Therefore, the problem underlying the present invention is to provide novel compounds having antibacterial and/or antiproliferative activity, especially compounds having antimicrobial activity against resistant or multiresistant Gram-positive bacteria.

### SUMMARY AND DESCRIPTION OF THE INVENTION

The present invention relates to a compound of the general formula (I):

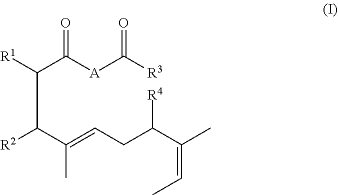

or a pharmacologically acceptable salt thereof, wherein

A represents a group of the formula:

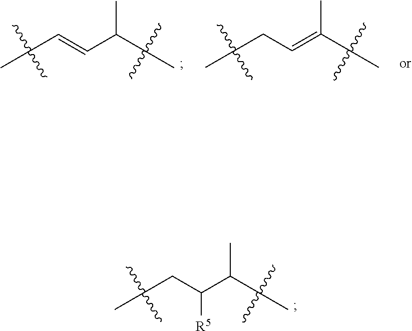

R¹, R², R³ and, if present, R⁵ each independently represents a hydrogen atom; a halogen atom; a hydroxyl group; an amino group; a mercapto group; a C₁-C₁₂ alkyl group; a C₂-C₁₂ alkenyl group; a C₂-C₁₂ alkynyl group; a heteroalkyl group containing 1 to 11 carbon atoms; a C₃-C₁₀ cycloalkyl group, a heterocycloalkyl group containing 3 to 10 ring atoms, a (C₁-C₆)alkyl-(C₃-C₇)cycloalkyl group, a (C₁-C₆)heteroalkyl-(C₃-C₇)cycloalkyl group, a C₆-C₁₄ aryl group, a heteroaryl group containing 5 to 14 ring atoms, an ar-(C₁-C₆)alkyl group, or a heteroar-(C₁-C₆)alkyl group containing 5 to 10 ring atoms;

R⁴ represents a hydrogen atom; a halogen atom; a hydroxyl group; an amino group; a C₁-C₁₂ alkyl group; or a heteroalkyl group containing 1 to 11 carbon atoms; or

R⁴ and R³ are taken together to form an oxygen or sulphur atom, or a group —NH—.

Compounds are usually described herein using standard nomenclature or the definitions presented below. For compounds having asymmetric centers, it should be understood that (unless otherwise specified) all of the optical isomers and mixtures thereof are encompassed. Compounds with two or more asymmetric elements can also be present as mixtures of diastereomers. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope (i.e., an atom having the same atomic number but a different mass number). By way of general example, and without limitation, isotopes of hydrogen include tritium and deuterium and isotopes of carbon include ¹¹C, ¹³C, and ¹⁴C.

Compounds according to the formulas provided herein, which have one or more stereogenic centers, have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single enantiomers (optically active forms) can be obtained by asymmetric synthesis, synthesis from optically pure precursors or by resolution of the racemates. Resolution of the racemates can be accomplished, for example, by conventional methods such as crystallization in the presence of a resolving agent, or chromatography, using, for example a chiral HPLC column.

Certain compounds are described herein using a general formula that includes variables, e.g. R¹, R², R³, and R⁴. Unless otherwise specified, each variable within such a formula is defined independently of any other variable, and any variable that occurs more than one time in a formula is defined independently at each occurrence. Thus, for example, if a group is shown to be substituted with 0-2 R*, the group may be unsubstituted or substituted with up to two R* groups and R* at each occurrence is selected independently from the definition of R*. Also, combinations of substituents and/or variables are permissible only if such combinations result in stable compounds, i.e., compounds that can be isolated, characterized and tested for biological activity.

As used herein a wording defining the limits of a range of length such as, e. g., “from 1 to 5” means any integer from 1 to 5, i. e. 1, 2, 3, 4 and 5. In other words, any range defined by two integers explicitly mentioned is meant to comprise and disclose any integer defining said limits and any integer comprised in said range. For example, the term “C₁-C₆” refers to 1 to 6, i.e. 1, 2, 3, 4, 5 or 6, carbon atoms.

A “pharmacologically acceptable salt” of a compound disclosed herein is an acid or base salt that is generally considered in the art to be suitable for use in contact with the tissues of human beings or animals without excessive toxicity or carcinogenicity, and preferably without irritation, allergic response, or other problem or complication. Such pharmaceutical salts include mineral and organic acid salts of basic residues such as amines, as well as alkali or organic salts of acidic residues such as carboxylic acids.

Suitable pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is any integer from 0 to 4 (i.e., 0, 1, 2, 3, or 4) and the like. Similarly, pharmaceutically acceptable cations include, but are not limited to sodium, potassium, calcium, aluminum, lithium and ammonium. Those of ordinary skill in the art will recognize further pharmacologically acceptable salts for the compounds provided herein. In general, a pharmacologically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of nonaqueous media, such as ether, ethyl acetate, ethanol, isopropanol or acetonitrile, is preferred.

It will be apparent that each compound of formula (I) may, but need not, be present as a hydrate, solvate or non-covalent complex. In addition, the various crystal forms and polymorphs are within the scope of the present invention, as are prodrugs of the compounds of formula (I) provided herein.

A “prodrug” is a compound that may not fully satisfy the structural requirements of the compounds provided herein, but is modified in vivo, following administration to a subject or patient, to produce a compound of formula I provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

A “substituent,” as used herein, refers to a molecular moiety that is covalently bonded to an atom within a molecule of interest. For example, a “ring substituent” may be a moiety such as a halogen, alkyl group, haloalkyl group, hydroxy, cyano, amino, nitro, mercapto, or other substituent described herein that is covalently bonded to an atom (preferably a carbon or nitrogen atom) that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e. a compound that can be isolated, characterized and tested for biological activity. When a substituent is oxo (i.e., ═O), then 2 hydrogens on the atom are replaced. An oxo group that is a substituent of an aromatic carbon atom results in a conversion of —CH— to —C(═O)— and a loss of aromaticity. For example a pyridyl group substituted by oxo is a pyridone.

As used herein, “comprising”, “including”, “containing”, “characterized by”, and grammatical equivalents thereof are inclusive or open-ended terms that do not exclude additional, unrecited elements or method steps. “Comprising”, etc. is to be interpreted as including the more restrictive term “consisting of”.

As used herein, “consisting of” excludes any element, step, or ingredient not specified in the claim.

When trade names are used herein, it is intended to independently include the trade name product formulation, the generic drug, and the active pharmaceutical ingredient(s) of the trade name product.

In general, unless defined otherwise, technical and scientific terms used herein have the same meaning as commonly understood by one of ordinary skill in the art to which this invention belongs, and are consistent with general textbooks and dictionaries.

The expression “optionally substituted” refers to groups in which one or more hydrogen atoms, e.g. 1 to 5 hydrogen atoms, and more preferably 1 to 3 hydrogen atoms, have been replaced each independently of the others by fluorine, chlorine, bromine or iodine atoms or by OH, ═O, SH, ═S, NH₂, ═NH, CN or NO₂ groups. The expression “optionally substituted” refers furthermore to groups in which one or more hydrogen atoms, e.g. 1 to 5 hydrogen atoms, and more preferably 1 to 3 hydrogen atoms, have been replaced each independently of the others by unsubstituted C₁-C₆alkyl, (C₁-C₆) haloalkyl (e.g. a fluoromethyl, trifluoromethyl, chloromethyl, (1- or 2-)haloethyl (e.g. (1- or 2-) chloroethyl), or (2- or 3-) halopropyl (e.g. (2- or 3-) fluoropropyl) group), (C₁-C₆) hydroxyalkyl (e.g. a hydroxymethyl, (1- or 2-)hydroxyethyl, or (2- or 3-) hydroxypropyl group), unsubstituted C₂-C₆alkenyl, unsubstituted C₂-C₆alkynyl, unsubstituted C₁-C₆heteroalkyl, unsubstituted C₃-C₇cycloalkyl, unsubstituted C₂-C₇heterocycloalkyl, unsubstituted C₆-C₁₀aryl, unsubstituted C₁-C₈heteroaryl, unsubstituted C₇-C₁₂aralkyl or unsubstituted C₂-C₁₁heteroaralkyl groups.

The expression alkyl or alkyl group denotes a saturated, straight-chain or branched hydrocarbon group that contains from 1 to 20 carbon atoms, preferably from 1 to 12 carbon atoms, more preferably from 1 to 6 carbon atoms, wherein said alkyl group may be optionally substituted. Examples of an unsubstituted alkyl group include a methyl, ethyl, propyl, isopropyl, n-butyl, iso-butyl, sec-butyl, tert-butyl, n-pentyl, n-hexyl, 2,2-dimethylbutyl, and n-octyl group, and examples of an optionally substituted alkyl group include haloalkanes, e.g. a trifluoromethyl group, or a difluoromethyl group; a hydroxymethyl group, 2-hydroxyethyl group, and a methoxymethyl group.

The expression alkenyl or alkenyl group refers to an at least partially unsaturated, straight-chain or branched hydrocarbon group that contains one or more double bond(s) and from 2 to 20 carbon atoms, preferably from 2 to 12 carbon atoms, more preferably from 2 to 6 carbon atoms, wherein said alkenyl group may be optionally substituted. Examples of an unsubstituted alkenyl group include an ethenyl (vinyl), propenyl (allyl), iso-propenyl, butenyl, isoprenyl and hex-2-enyl group. Preferably, an alkenyl group has one or two, especially one, double bond(s).

The expression alkynyl or alkynyl group refers to an at least partially unsaturated, straight-chain or branched hydrocarbon group that contains one or more triple bond(s) and from 2 to 20 carbon atoms, preferably from 2 to 12 carbon atoms, more preferably from 2 to 6, e.g. 2, 3 or 4, carbon atoms, wherein said alkynyl group may be optionally substituted. Examples of an unsubstituted alkynyl group include an ethynyl (acetylenyl), propynyl, butynyl or propargyl group. Preferably, an alkynyl group has one or two, especially one, triple bond(s).

As used herein, the expression “heteroalkyl” or heteroalkyl group also includes heteroalkenyl (group) and heteroalkynyl (group), and accordingly refers to an alkyl, alkenyl or alkynyl (straight chain or branched) group as defined above, in which one or more, preferably 1, 2, 3 or 4, carbon atoms have been replaced each independently of the others by an oxygen, nitrogen, phosphorus, boron, selenium, silicon or sulphur atom, preferably by an oxygen, sulphur or nitrogen atom, or by an SO or SO₂ group, wherein said heteroalkyl group may be optionally substituted. As a result, the expression heteroalkyl encompasses groups containing 1 to 19 carbon atoms, preferably from 1 to 11 carbon atoms, more preferably from 1 to 5, e.g. 1, 2, 3 or 4, carbon atoms, and accordingly may also be referred to as C₁-C₁₉, C₁-C₁₁, and C₁-C₅ heteroalkyl, respectively. Further, the expression heteroalkyl also encompasses groups derived from a carboxylic acid, such as, for example, acyl, acylalkyl, alkoxycarbonyl, acyloxy, acyloxyalkyl, carboxyalkylamide or alkoxycarbonyloxy. Examples of heteroalkyl groups are alkylamino, dialkylamino, alkylaminoalkyl, dialkylaminoalkyl, acylalkyl, alkoxycarbonyl, alkylcarbamoyl, alkylamido, alkylcarbamoylalkyl, alkylamidoalkyl, alkylcarbamoyloxyalkyl, alkylureidoalkyl, alkoxycarbonyloxy, alkoxy, or alkoxyalkyl. The expression alkoxy or alkoxy group refers to an alkyl group, in which one or more non-adjacent CH₂ group(s) are replaced by oxygen, wherein the alkyl moiety of the alkoxy group may be optionally substituted. The expression alkylthio or alkylthio group refers to an alkyl group, in which one or more non-adjacent CH₂ group(s) are replaced by sulfur, wherein the alkyl moiety of the alkylthio group may be optionally substituted.

Further examples of heteroalkyl groups are groups of formulae: R^{a}—O—Y^{a}—, R^{a}—S—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; R^{d} being a hydrogen atom, a C₁-C₆ alkyl, a C₂-C₆ alkenyl or a C₂-C₆ alkynyl group and Y^{a} being a direct bond, a C₁-C₆ alkylene, a C₂-C₆ alkenylene or a C₂-C₆ alkynylene group. Specific examples of heteroalkyl groups include acyl, methoxy, trifluoromethoxy, ethoxy, n-propyloxy, isopropyloxy, tert-butyloxy, methoxymethyl, ethoxymethyl, methoxyethyl, methylamino, ethylamino, dimethylamino, diethylamino, isopropylethylamino, methylaminomethyl, ethylaminomethyl, diisopropyl-aminoethyl, dimethylaminomethyl, dimethylaminoethyl, acetyl, propionyl, butyryloxy, acetyloxy, methoxycarbonyl, ethoxycarbonyl, isobutyrylamino-methyl, N-ethyl-N-methylcarbamoyl, N-methylcarbamoyl, cyano, nitrile, isonitrile, thiocyanate, isocyanate, isothiocyanate and alkylnitrile.

The expression cycloalkyl or cycloalkyl group refers to a saturated or partially unsaturated cyclic group that contains one or more rings (preferably 1 or 2), containing from 3 to 14 ring carbon atoms, preferably from 3 to 10 (more preferably 3, 4, 5, 6 or 7) ring carbon atoms, wherein the cycloalkyl group may be optionally substituted. In an embodiment a partially unsaturated cyclic group has one, two or more double bonds, such as a cycloalkenyl group.

Specific examples of an unsubstituted cycloalkyl group are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, cyclopentylcyclohexyl, and a cyclohex-2-enyl group.

The expression heterocycloalkyl or heterocycloalkyl group refers to a cycloalkyl group as defined above in which one or more, preferably 1, 2 or 3, ring carbon atoms have been replaced each independently of the others by an oxygen, nitrogen, or sulphur atom (preferably oxygen or nitrogen), or by a SO or SO2 group. A heterocycloalkyl group has preferably 1 or 2 ring(s) containing from 3 to 10 (more preferably 3, 4, 5, 6 or 7, and most preferably 5, 6 or 7) ring atoms. Examples are an aziridinyl, oxiranyl, thiiranyl, oxaziridinyl, dioxiranyl, azetidinyl, oxetanyl, thietanyl, diazetidinyl, dioxetanyl, dithietanyl, pyrrolidinyl, tetrahydrofuranyl, thiolanyl, phospholanyl, silolanyl, azolyl, thiazolyl, isothiazolyl, imidazolidinyl, pyrazolidinyl, oxazolidinyl, isoxazolidinyl, thiazolidinyl, isothiazolidinyl, dioxolanyl, dithiolanyl, piperazinyl, morpholinyl, thiomorpholinyl, trioxanyl, azepanyl, oxepanyl, thiepanyl, homopiperazinyl, or urotropinyl group. Further examples are a 2-pyrazolinyl group, and also a lactam, a lactone and a cyclic imide. The heterocycloalkyl group can be optionally substituted, and may be saturated or mono-, di- or tri-unsaturated. As a result, the expression heterocycloalkyl group also encompasses a group derived from a carbohydrate or saccharide, such as furanoses or pentoses, e.g. arabinose, ribose, xylose, lyxose or desoxyribose, or pyranoses/hexoses or derivatives thereof, e.g. allose, altrose, glucose, mannose, gulose, idose, galactose, talose, 6-carboxy-D-glucose, 6-carboxy-D-galactose, N-acetylchitosamine, glucosamine, N-acetylchondrosamin, fucose, rhamnose, or chinovose.

The expression alkylcycloalkyl or alkylcycloalkyl group refers to a group containing both cycloalkyl and also an alkyl, alkenyl or alkynyl group in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two ring systems having from 3 to 10 (preferably 3, 4, 5, 6 or 7) carbon atoms, and one or two alkyl, alkenyl or alkynyl groups having 1 or 2 to 6 carbon atoms, the cyclic groups being optionally substituted.

The expression heteroalkylcycloalkyl or heteroalkylcycloalkyl group refers to alkylcycloalkyl groups as defined above in which one or more (preferably 1, 2 or 3) carbon atoms have been replaced each independently of the others by an oxygen, nitrogen, silicon, selenium, phosphorus or sulphur atom (preferably oxygen, sulphur or nitrogen). A heteroalkylcycloalkyl group preferably contains 1 or 2 ring systems having from 3 to 10 (preferably 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylhetero-cycloalkyl and heteroalkylheterocycloalkenyl, the cyclic groups being optionally substituted and saturated or mono-, di- or tri-unsaturated.

The expressions aryl, Ar or aryl group refer to an aromatic group that contains one or more rings containing from 6 to 14 ring carbon atoms (C₆-C₁₄), preferably from 6 to 10 (C₆-C₁₀), more preferably 6 ring carbon atoms, wherein the aryl group may be optionally substituted. Examples of an unsubstituted aryl group include a phenyl, naphthyl, biphenyl, or indanyl group.

The expression heteroaryl refers to an aromatic group that contains one or more rings containing from 5 to 14 ring atoms, preferably from 5 to 10 (more preferably 5 or 6) ring atoms, and contains one or more (preferably 1, 2, 3 or 4) oxygen, nitrogen, phosphorus or sulphur ring atoms (preferably O, S or N), wherein the heteroaryl group may be optionally substituted. Examples of an unsubstituted. Examples of an unsubstituted heteroaryl group include 2-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl.

The expression aralkyl or aralkyl group refers to a group containing both aryl and also alkyl, alkenyl, alkynyl and/or cycloalkyl groups in accordance with the above definitions, wherein the aralkyl group may be optionally substituted. As a result, the expression aralkyl group encompasses groups such as, for example, arylalkyl, arylalkenyl, arylalkynyl, arylcycloalkyl, arylcycloalkenyl, alkylarylcycloalkyl and alkylarylcycloalkenyl groups, wherein all of said groups may be optionally substituted. Specific examples of an unsubstituted aralkyl group include toluene, xylene, mesitylene, styrene, benzyl chloride, o-fluorotoluene, 1H-indene, tetralin, dihydronaphthalene, indanone, phenylcyclopentyl, cumene, cyclohexyl-phenyl, fluorene and indan. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl or heteroaralkyl group refers to an aralkyl group as defined above in which one or more (preferably 1, 2, 3 or 4) carbon atoms have been replaced each independently of the others by an oxygen, nitrogen, silicon, selenium, phosphorus, boron or sulphur atom (preferably oxygen, sulphur or nitrogen), that is to say to groups containing both aryl or heteroaryl and also alkyl, alkenyl, alkynyl and/or heteroalkyl and/or cycloalkyl and/or heterocycloalkyl groups in accordance with the above definitions, wherein the heteroaralkyl group may be optionally substituted. A heteroaralkyl group preferably contains one or two aromatic ring systems (1 or 2 rings) containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, 1, 2, 3 or 4 of those carbon atoms having been replaced each independently of the others by oxygen, sulphur or nitrogen atoms. Examples of heteroaralkyl groups are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroaryl-heteroalkyl, heteroarylcycloalkyl, heteroarylcycloalkenyl, heteroarylheterocycloalkyl, hetero-arylheterocycloalkenyl, heteroarylalkylcycloalkyl, heteroarylalkylheterocycloalkenyl, hetero-arylheteroalkylcycloalkyl, heteroarylheteroalkylcycloalkenyl, heteroalkylheteroarylalkyl and heteroarylheteroalkylheterocycloalkyl groups; all of which groups may be optionally substituted and the cyclic moieties of said groups being saturated or mono-, di- or tri-unsaturated. Specific examples of a heteroaralkyl group include a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

The expression “halogen” or “halogen atom” as preferably used herein means fluorine, chlorine, bromine, or iodine.

The activity and more specifically the bioactivity of the compounds according to the present invention can be assessed using appropriate assays known to those skilled in the art, e.g. in vitro or in vivo assays. For instance, the antimicrobial activity against Gram-positive bacteria and the cytotoxic activity against cell lines may be determined via a growth inhibition assay, such as the assays provided in Examples 2 and 3 below, which are thus embodiments of standard in vitro assays.

Preferred is a compound of formula (I), or a pharmacologically acceptable salt thereof, wherein

R¹, R² and, if present, R⁵ each independently represents a hydrogen atom; a halogen atom; a hydroxyl group; an amino group; a mercapto group; a C₁-C₁₂ alkyl group; a C₂-C₁₂ alkenyl group; a C₂-C₁₂ alkynyl group; a heteroalkyl group containing 1 to 11 carbon atoms; a C₃-C₁₀ cycloalkyl group, a heterocycloalkyl group containing 3 to 10 ring atoms, a (C₁-C₆)alkyl-(C₃-C₇)cycloalkyl group, a (C₁-C₆)heteroalkyl-(C₃-C₇)cycloalkyl group, a C₆-C₁₄ aryl group, a heteroaryl group containing 5 to 14 ring atoms, an ar-(C₁-C₆)alkyl group, or a heteroar-(C₁-C₆)alkyl group containing 5 to 10 ring atoms; and R³ and R⁴ are taken together to form an oxygen atom.

Further preferred is a compound of formula (I), or a pharmacologically acceptable salt thereof, wherein

R¹ and R² each independently represents a hydrogen atom; a halogen atom; a hydroxyl group; a heteroalkyl group containing 1 to 11 carbon atoms; or a heterocycloalkyl group containing 3 to 10 ring atoms. R³, R⁴ and, if present R⁵, are as defined above.

In the compound of formula (I), R¹ can represent a hydrogen atom; a hydroxyl group or a heteroalkyl group containing 1 to 11 carbon atoms; and R² can represent a hydroxyl group; or a heterocycloalkyl group containing 3 to 10 ring atoms.

In the compound of formula (I), R³ and R⁴ are preferably taken together to form an oxygen atom. Preferably, R¹ can represent a heteroalkyl group containing 1 to 11 carbon atoms. R² can preferably represent a heterocycloalkyl group containing 3 to 10 ring atoms.

In the compound of formula (I), R⁵, if present, can preferably represent a hydrogen atom; a halogen atom; a hydroxyl group; an amino group; a C₁-C₁₂ alkyl group; a C₂-C₁₂ alkenyl group; a C₂-C₁₂ alkynyl group; or a heteroalkyl group containing 1 to 11 carbon atoms.

The compound of formula (I) may be selected from the group consisting of:

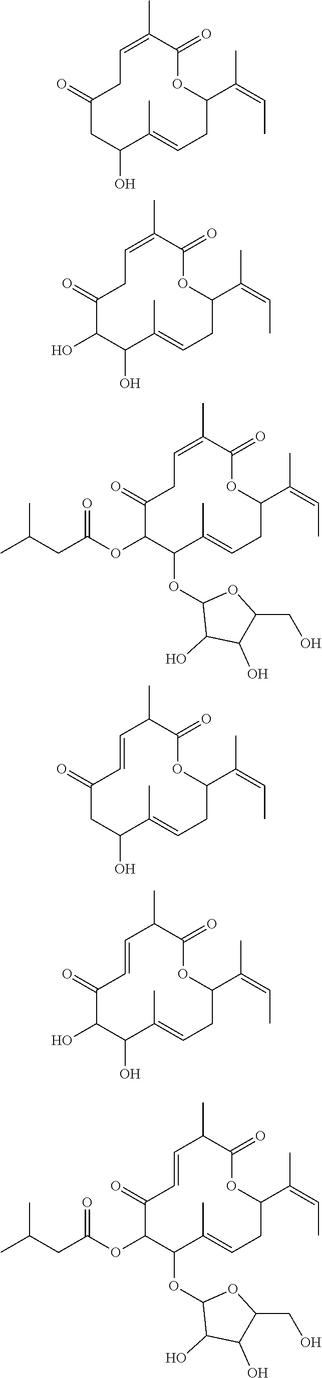

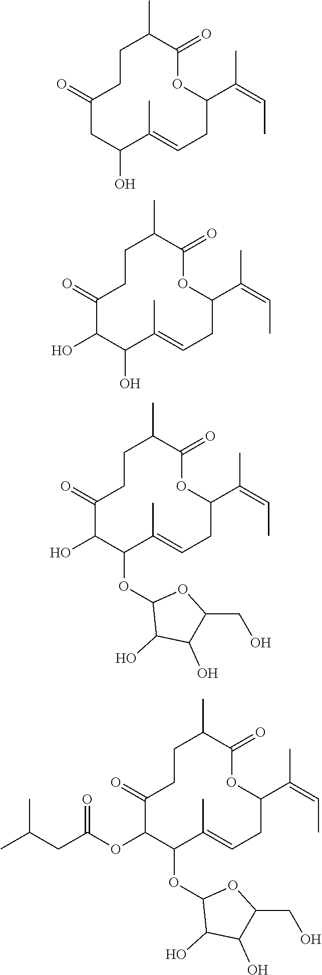

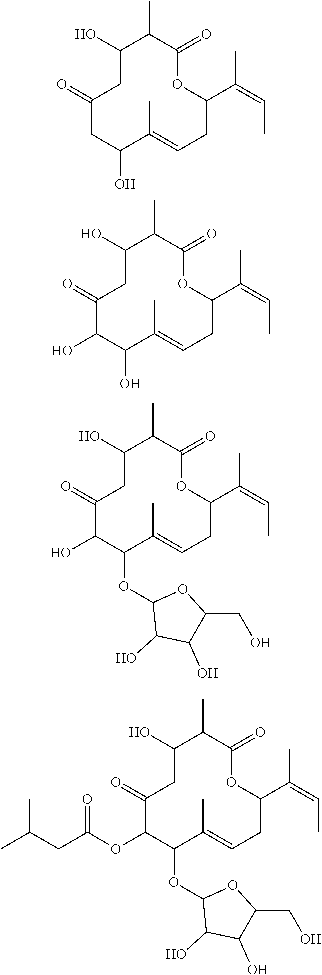

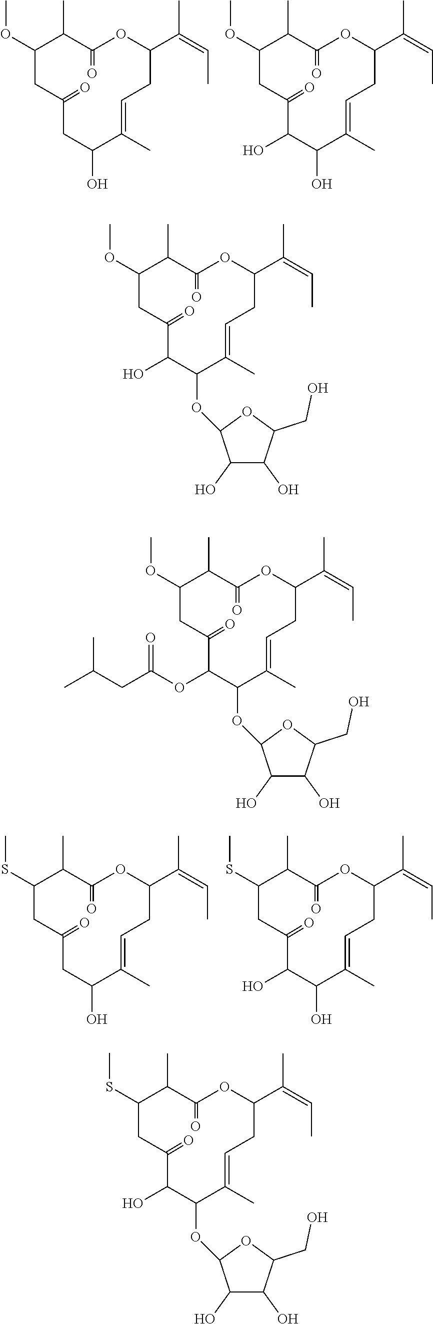

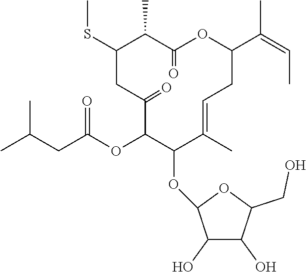 [SMILES: C/C=C(/C)C1C/C=C(\C)C(OC2OC(CO)C(O)C2O)C(OC(=O)CC(C)C)C(=O)CC(SC)[C@H](C)C(=O)O1]

In a preferred embodiment, the compound of formula (I) is compound (1)—disciformycin A; compound (2)—disciformycin B; compound (3)—gulmirecin A; compound (4)—gulmirecin B; compound (5)—disciformycin C, or compound (6)—disciformycin D having the structure as depicted below:

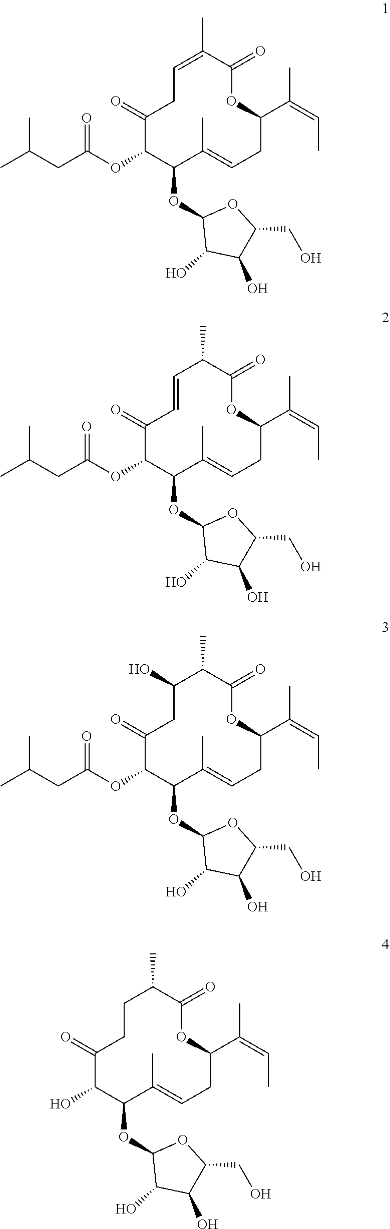

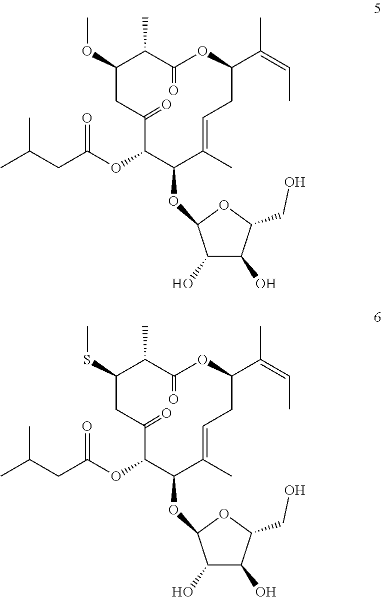

The therapeutic use of a compound of formula (I), of a pharmacologically acceptable salt, solvate or hydrate thereof, and also of a formulation and/or pharmaceutical composition containing the same is within the scope of the present invention. The present invention also relates to the use of a compound of formula (I) as active ingredient in the preparation or manufacture of a medicament.

A pharmaceutical composition according to the present invention comprises at least one compound of formula (I) and, optionally, one or more carrier substance(s), excipient(s) and/or adjuvant(s). Pharmaceutical compositions may additionally comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Furthermore, one or more other active ingredients may (but need not) be included in the pharmaceutical compositions provided herein. For instance, the compounds of the invention may advantageously be employed in combination with another antibiotic or antifungal agent, an anti-viral agent, an anti histamine, a non-steroidal anti-inflammatory drug, a disease modifying anti-rheumatic drug, another cytostatic drug, a drug with smooth muscle activity modulatory activity or mixtures of the aforementioned.

A pharmaceutical composition may be formulated for any appropriate route of administration, including, for example, parenteral administration. The term parenteral as used herein includes subcutaneous, intradermal, intravascular such as, e.g., intravenous, intramuscular, spinal, intracranial, intrathecal, intraocular, periocular, intraorbital, intrasynovial and intraperitoneal injection, as well as any similar injection or infusion technique.

Carrier substances are, for example, cyclodextrins such as hydroxypropyl β-cyclodextrin, micelles or liposomes, excipients and/or adjuvants. Customary excipients include, for example, inert diluents such as, e.g., calcium carbonate, sodium carbonate, lactose, calcium phosphate or sodium phosphate, granulating and disintegrating agents such as, e.g., corn starch or alginic acid, binding agents such as, e.g., starch, gelatin or acacia, and lubricating agents such as, e.g., magnesium stearate or stearic acid. Examples of adjuvants are aluminum hydroxide, aluminum phosphate, calcium phosphate hydroxide, paraffin oil, squalene, thimerosal, detergents, Freund's complete adjuvant, or Freund's incomplete adjuvant.

For the prevention and/or treatment of bacterial infections, especially infections with Gram-positive bacteria, or other conditions, e.g. a prolifertive disease, the dose of the biologically active compound according to the invention may vary within wide limits and may be adjusted to individual requirements. Active compounds according to the present invention are generally administered in a therapeutically effective amount. The expression “therapeutically effective amount” denotes a quantity of the compound(s) that produces a result that in and of itself helps to ameliorate, heal, or cure the respective condition or disease. Preferred doses range from about 0.1 mg to about 140 mg per kilogram of body weight per day (about 0.5 mg to about 7 g per patient per day). The daily dose may be administered as a single dose or in a plurality of doses. The amount of active ingredient that may be combined with the carrier materials to produce a single dosage form will vary depending upon the host treated and the particular mode of administration. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient) and the severity of the particular disease undergoing therapy.

The invention further relates to a combination preparation containing at least one compound according to the invention and at least one further active pharmaceutical ingredient. The combination preparation of the invention can be used as a medicament, in particular in the treatment or prophylaxis of bacterial infections with Gram-positive bacteria, such as an infection with Bacillus subtilis, Mycobacterium aurum, Mycobacterium smegmatis, Mycobacterium vaccae, Paenibacillus polymyxa, or staphylococci, e.g. Staphylococcus aureus, Staphylococcus aureus Newman, Staphylococcus aureus (MRSA), Staphylococcus aureus N315 (MRSA), Staphylococcus aureus Mu50 (MRSA/VRSA), Staphylococcus auricularis, and Staphylococcus carnosus.

Preferably, in the combination preparation of the invention the further active pharmaceutical ingredient is another antibiotic. The other antibiotic can be selected from the group consisting of β-lactam antibiotics, including penams, carbapenams, oxapenams, penems, carbapenems, monobactams, cephems, carbacephems, oxacephems, and monobactams; aminoglycoside antibiotics, including amikacin, arbekacin, astromicin, bekanamycin, dibekacin, framycetin, gentamicin, hygromycin B, isepamicin, kanamycin, neomycin, netilmicin, paromomycin, paromomycin sulfate, ribostamycin, sisomicin, spectinomycin, streptomycin, tobramycin, and verdamicin; quinolone antibiotics, including ciprofloxacin, enoxacin, gatifloxacin, grepafloxacin, levofloxacin, lomefloxacin, moxifloxacin, nalidixic acid, norfloxacin, ofloxacin, sparfloxacin, temafloxacin, and trovafloxacin; or glycopeptide antibiotics, e.g. vancomycin, telavancin, bleomycin, ramoplanin, and decaplanin; linezolid; or daptomycin.

Preferred compounds of the invention will have certain pharmacological properties. Such properties include, but are not limited to, bioavailability (especially with regard to oral administration), metabolic stability and sufficient solubility, such that the dosage forms can provide therapeutically effective levels of the compound in vivo.

The compound according to the invention as well as the pharmaceutical composition or combination preparation according to the invention can be used as a medicament, which can be administered to a patient (e.g. parenterally to a human or an other mammal), with dosages as described herein, and will be present within at least one body fluid or tissue of the patient. As used herein, the term “treatment” encompasses both disease-modifying treatment and symptomatic treatment, either of which may be prophylactic, i.e., before the onset of symptoms, in order to prevent, delay or reduce the severity of symptoms, or therapeutic, i.e., after the onset of symptoms, in order to reduce the severity and/or duration of symptoms. In particular, the conditions or diseases that can be ameliorated, prevented or treated using a compound of formula (I), a pharmaceutical composition or a combination preparation according to the invention include bacterial infections and antiproliferative diseases, e.g. cancer. Thus, the present invention also provides methods for treating patients suffering from said diseases. A method for the treatment of a subject which is in need of such treatment, comprises the administration of a compound, a pharmaceutical composition, or a combination preparation according to the invention. The term “subject” refers to patients, including, but not limited to primates (especially humans), domesticated companion animals (such as dogs, cats, horses) and livestock (such as cattle, pigs, sheep).

A recombinant polyketide synthase (PKS) capable of synthesizing a compound according to general formula (I), wherein the PKS comprises at least one polypeptide, or a functional variant thereof, according to any one of SEQ ID NOs. 11 to 19 or SEQ ID NOs. 34 to 46.

The term “functional variant” as used herein denotes a polypeptide having a sequence that is at least 85%, 90%, 95% or 99% identical to a polypeptide sequence described herein. A “functional variant” of a polypeptide may retain amino acids residues recognized as conserved for the polypeptide in nature, and/or may have non-conserved amino acid residues. Amino acids can be, relative to the native polypeptide, substituted (different), inserted, or deleted, but the variant has generally similar (enzymatic) activity or function as compared to a polypeptide described herein. A “functional variant” may be found in nature or be an engineered mutant (recombinant) thereof.

The term “identity” refers to a property of sequences that measures their similarity or relationship. Identity is measured by dividing the number of identical residues by the total number of residues and multiplying the product by 100.

The terms “protein”, “polypeptide”, “peptide” as used herein define an organic compound made of two or more amino acid residues arranged in a linear chain, wherein the individual amino acids in the organic compound are linked by peptide bonds, i.e. an amide bond formed between adjacent amino acid residues. By convention, the primary structure of a protein is reported starting from the amino-terminal (N) end to the carboxyl-terminal (C) end.

Preferably, the PKS enzyme of the invention is a not naturally occurring PKS. The PKS of the invention may also be a hybrid PKS comprising modules, domains, and/or portions thereof, or functional variants thereof, from two or more PKSs or from one or more nonribosomal peptide-synthetase(s) (NRPSs). The PKS of the invention preferably includes one or more of the open reading frame(s) (ORF(s)) indicated in Table A or Table B below. Preferably, the PKS of the invention includes 1, 2, 3, 4, 5 or 6 ORF(s) selected from the group consisting of difB, difC, difD, difE, difF, and difG; or 1, 2, 3, 4, 5, or 6 ORF(s) selected from the group consisting of gulA, gulB, gulC, gulD, gulE, and gulF.

TABLE A
Annotated ORFs in the dif gene cluster, closest homologues
found by BLAST-search, and SEQ ID NOs. of the nucleotide
and corresponding amino acid sequence.

| ORF | closest homologue | source organism | SEQ ID NO |
|---|---|---|---|
| difA | cytochrome 450 | P. mucilaginosus 3016 | 2, 11 |
| difB | polyketide synthase | S. aurantiaca | 3, 12 |
| difC | polyketide synthase | M. xanthus Dk 1622 | 4, 13 |
| difD | polyketide synthase | S. aurantiaca | 5, 14 |
| difE | polyketide synthase | N. punctiforme PCC 73102 | 6, 15 |
| difF | polyketide synthase | S. cellulosum So ce56 | 7, 16 |
| difG | polyketide synthase | S. aurantiaca | 8, 17 |
| difH | hypothetical protein | S. cellulosum So ce56 | 9, 18 |
| difI | CRP-like protein | M. fulvus HW-11 | 10, 19 |

TABLE B
Annotated ORFs in the gul gene cluster, closest homologues found by
BLAST-search, and SEQ ID NOs. of the nucleotide and
corresponding amino acid sequence.

| ORF | closest homologue | source organism | SEQ ID NO |
|---|---|---|---|
| gulG | cytochrome P450 | P. mucilaginosus 3016 | 21, 34 |
| gulA | polyketide synthase | S. aurantiaca Sg a15 | 22, 35 |
| gulB | polyketide synthase | S. aurantiaca Sg a15 | 23, 36 |
| gulC | polyketide synthase | M. stipitatus DSM 14675 | 24, 37 |
| gulD | polyketide synthase | S. aurantiaca Sg a15 | 25, 38 |
| gulE | polyketide synthase | S. aurantiaca DW4/3-1 | 26, 39 |
| gulF | polyketide synthase | S. cellulosum So ce38 | 27, 40 |
| gulH | hypothetical protein | S. cellulosum So ce56 | 28, 41 |
| gulI | cAMP-binding protein | Myxococcus sp. | 29, 42 |
| | | (contaminant ex | |
| | | DSM 436) | |
| gulJ | hypothetical protein | S. cellulosum So0157-2 | 30, 43 |
| gulR1 | LysR family | C. fuscus DSM 2262 | 31, 44 |
| | transcriptional regulator | | |
| gulK | sui1 family protein | S. aurantiaca DW4/3-1 | 32, 45 |
| gulR2 | TetR family transcriptional | K. racemifer DSM 44963 | 33, 46 |
| | regulator | | |

The present invention also provides isolated, synthetic or recombinant nucleic acids that encode PKSs of the invention. Said nucleic acids include nucleic acids that include a portion or all of a PKS of the invention, nucleic acids that further include regulatory sequences, such as promoter and translation initiation and termination sequences, and can further include sequences that facilitate stable maintenance in a host cell, i.e., sequences that provide the function of an origin of replication or facilitate integration into host cell chromosomal or other DNA by homologous recombination.

Preferably, the invention relates to an isolated, synthetic or recombinant nucleic acid comprising:

(i) a sequence encoding a PKS of the invention, wherein the sequence has a sequence identity to the full-length sequence of SEQ ID NO: 1 or SEQ ID NO: 20 from at least 85%, 90%, 95%, 96%, 97%, 98%, 98.5%, 99%, or 99.5% to 100%;

(ii) a sequence encoding a portion of a PKS of the invention, wherein the sequence has a sequence identity to the full-length sequence of any of SEQ ID NOs. 2 to 10 or SEQ ID NOs. 21 to 33 from at least 85%, 90%, 95%, 96%, 97%, 98%, 98.5%, 99%, or 99.5% to 100%;

(iii) a sequence completely complementary to any nucleic acid sequence of (i) or (ii); or

(iv) a sequence encoding a polypeptide according to any of SEQ ID NOs. 11 to 19 or SEQ ID NOs. 34 to 46.

The phrases “nucleic acid” or “nucleic acid sequence” as used herein refer to an oligonucleotide, nucleotide, polynucleotide, or to a fragment of any of these, to DNA of genomic or synthetic origin which may be single-stranded or double-stranded and may represent a sense or antisense strand, natural or synthetic in origin. “Oligonucleotide” includes either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide can ligate to a fragment that has not been dephosphorylated. A “coding sequence” of or a “nucleotide sequence encoding” a particular polypeptide or protein, is a nucleic acid sequence which is transcribed and translated into a polypeptide or protein when placed under the control of appropriate regulatory sequences. The nucleic acids used to practice this invention may be isolated from a variety of sources, genetically engineered, amplified, and/or expressed/generated recombinantly. Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, et al, Molecular Cloning: A Laboratory Manual, 2nd Ed., Cold Spring Harbor, N. Y., VoIs. 1-3, (1989); Current Protocolsin Molecular Biology, Ausubel, ed. John Wiley & Sons, Inc., New York (1997); Laboratory Techniques In Biochemistry And Molecular Biology: Hybridization With Nucleic Acid Probes, Part I. Theory and Nucleic Acid Preparation, Tijssen, ed. Elsevier, N.Y. (1993). A nucleic acid encoding a polypeptide of the invention is assembled in appropriate phase with a leader sequence capable of directing secretion of the translated polypeptide or fragment thereof.

The term “isolated” as used herein means that the material, e.g., a nucleic acid, a polypeptide, a vector, a cell, is removed from its original environment, e.g., the natural environment if it is naturally occurring. For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment.

The term “synthetic” as used herein means that the material, e.g. a nucleic acid, has been synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22: 1859.

The term “recombinant” means that the nucleic acid is adjacent to a “backbone” nucleic acid to which it is not adjacent in its natural environment. Backbone molecules according to the invention include nucleic acids such as cloning and expression vectors, self-replicating nucleic acids, viruses, integrating nucleic acids and other vectors or nucleic acids used to maintain or manipulate a nucleic acid insert of interest. Recombinant polypeptides of the invention, generated from these nucleic acids can be individually isolated or cloned and tested for a desired activity. Any recombinant expression system can be used, including bacterial, mammalian, yeast, insect or plant cell expression systems.

Also provided is a vector comprising at least one nucleic acid according to the invention. The vector may be a cloning vector, an expression vector or an artificial chromosome.

As used herein, the term “vector” refers to a nucleic acid molecule capable of transporting another nucleic acid to which it has been linked. Vectors, including cloning and expression vectors, comprise a nucleic acid of the invention or a functional equivalent thereof. Nucleic acids of the invention can be incorporated into a recombinant replicable vector, for example a cloning or expression vector. The vector may be used to replicate the nucleic acid in a compatible host cell. Thus, the invention also provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below. The vector into which the expression cassette or nucleic acid of the invention is inserted may be any vector which may conveniently be subjected to recombinant DNA procedures, and the choice of the vector will often depend on the host cell into which it is to be introduced. A variety of cloning and expression vectors for use with prokaryotic and eukaryotic hosts are described by Sambrook, et al, Molecular Cloning: A Laboratory Manual, 2nd Ed., Cold Spring Harbor, N. Y., (1989).

A vector according to the invention may be an autonomously replicating vector, i.e. a vector which exists as an extra-chromosomal entity, the replication of which is independent of chromosomal replication, e. g. a plasmid. Alternatively, the vector may be one which, when introduced into a host cell, is integrated into the host cell genome and replicated together with the chromosome(s) into which it has been integrated.

One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication, and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as cosmid, viral vectors (e.g., replication defective retroviruses, adenoviruses and adeno-associated viruses) and phage vectors which serve equivalent functions.

Vectors according to the invention may be used in vitro, for example for the production of RNA or used to transfect or transform a host cell.

A vector of the invention may comprise two or more, for example three, four or five, nucleic acids of the invention, for example for overexpression.

The recombinant expression vectors of the invention comprise a nucleic acid of the invention in a form suitable for expression of the nucleic acid in a host cell, which means that the recombinant expression vector includes one or more regulatory sequences, selected on the basis of the host cells to be used for expression, which is operationally linked to the nucleic acid sequence to be expressed.

Within a vector, such as an expression vector, “operationally linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell), i.e. the term “operationally linked” refers to a juxtaposition wherein the components described are in a relationship permitting them to function in their intended manner. A regulatory sequence such as a promoter, enhancer or other expression regulation signal “operationally linked” to a coding sequence is positioned in such a way that expression of the coding sequence is achieved under condition compatible with the control sequences or the sequences are arranged so that they function in concert for their intended purpose, for example transcription initiates at a promoter and proceeds through the DNA sequence encoding the polypeptide.

The term “regulatory sequence” or “control sequence” is intended to include promoters, operators, enhancers, attenuators and other expression control elements (e.g., polyadenylation signal). Such regulatory sequences are described, for example, in Goeddel; Gene Expression Technology: Methods in Enzymology 185, Academic Press, San Diego, Calif. (1990).

The term regulatory or control sequences includes those sequences which direct constitutive expression of a nucleotide sequence in many types of host cells and those which direct expression of the nucleotide sequence only in a certain host cell (e.g. tissue-specific regulatory sequences).

A vector or expression construct for a given host cell may thus comprise the following elements operationally linked to each other in a consecutive order from the 5′-end to 3′-end relative to the coding strand of the sequence encoding the polypeptide of the invention: (i) a promoter sequence capable of directing transcription of the nucleotide sequence encoding the polypeptide in the given host cell; (ii) optionally, a signal sequence capable of directing secretion of the polypeptide from the given host cell into a culture medium; (iii) optionally, a sequence encoding for a C-terminal, N-terminal or internal epitope tag sequence or a combination of the aforementioned allowing purification, detection or labeling of the polypeptide; (iv) a nucleic acid sequence of the invention encoding a polypeptide of the invention; and preferably also (v) a transcription termination region (terminator) capable of terminating transcription downstream of the nucleotide sequence encoding the polypeptide. Particular named bacterial promoters include lad, lacZ, T3, T7, SP6, K1F, tac, tet, gpt, lambda P_{R}, P_{L} and trp. Eukaryotic promoters include CMV immediate early, HSV thymidine kinase, early and late SV40, LTRs from retrovirus and mouse metallothionein-I. Selection of the appropriate vector and promoter is well within the level of ordinary skill in the art. Downstream of the nucleotide sequence according to the invention there may be a 3′ untranslated region containing one or more transcription termination sites (e. g. a terminator). The origin of the terminator is less critical. The terminator can, for example, be native to the DNA sequence encoding the polypeptide. Preferably, the terminator is endogenous to the host cell (in which the nucleotide sequence encoding the polypeptide is to be expressed). In the transcribed region, a ribosome binding site for translation may be present. The coding portion of the mature transcripts expressed by the constructs will include a translation initiating AUG (or TUG or GUG in prokaryotes) at the beginning and a termination codon appropriately positioned at the end of the polypeptide to be translated.

Enhanced expression of a polynucleotide of the invention may also be achieved by the selection of heterologous regulatory regions, e.g. promoter, secretion leader and/or terminator regions, which may serve to increase expression and, if desired, secretion levels of the protein of interest from the expression host and/or to provide for the inducible control of the expression of a polypeptide of the invention. It will be appreciated by those skilled in the art that the design of the expression vector can depend on such factors as the choice of the host cell to be transformed, the level of expression of protein desired, etc. The vectors, such as expression vectors, of the invention can be introduced into host cells to thereby produce proteins or peptides, encoded by nucleic acids as described herein.

The vectors, such as recombinant expression vectors, of the invention can be designed for expression of a portion or all of a PKS of the invention in prokaryotic or eukaryotic cells. For example, a portion or all of a PKS of the invention can be expressed in bacterial cells such as E. coli, Bacillus strains, insect cells (using baculovirus expression vectors), filamentous fungi, yeast cells or mammalian cells. Suitable host cells are discussed further in Goeddel, Gene Expression Technology: Methods in Enzymology 185, Academic Press, San Diego, Calif. (1990). Representative examples of appropriate hosts are described hereafter. Appropriate culture media and conditions for the above-described host cells are known in the art.

As set out above, the term “control sequences” or “regulatory sequences” is defined herein to include at least any component which may be necessary and/or advantageous for the expression of a polypeptide. Any control sequence may be native or foreign to the nucleic acid sequence of the invention encoding a polypeptide. Such control sequences may include, but are not limited to, a promoter, a leader, optimal translation initiation sequences (as described in Kozak, 1991, J. Biol. Chem. 266:19867-19870), a secretion signal sequence, a pro-peptide sequence, a polyadenylation sequence, a transcription terminator. At a minimum, the control sequences typically include a promoter, and transcriptional and translational stop signals. A stably transformed microorganism is one that has had one or more DNA fragments introduced such that the introduced molecules are maintained, replicated and segregated in a growing culture. Stable transformation may be due to multiple or single chromosomal integration(s) or by (an) extrachromosomal element(s) such as (a) plasmid vector(s). A plasmid vector is capable of directing the expression of polypeptides encoded by particular DNA fragments. Expression may be constitutive or regulated by inducible (or repressible) promoters that enable high levels of transcription of functionally associated DNA fragments encoding specific polypeptides.

Expression vectors of the invention may also include a selectable marker gene to allow for the selection of bacterial strains that have been transformed, e.g., genes which render the bacteria resistant to drugs such as chloramphenicol, erythromycin, kanamycin, neomycin, tetracycline, as well as ampicillin and other penicillin derivatives like carbenicillin. Selectable markers can also include biosynthetic genes, such as those in the histidine, tryptophan and leucine biosynthetic pathways.

The appropriate polynucleotide sequence may be inserted into the vector by a variety of procedures. In general, the polynucleotide sequence is ligated to the desired position in the vector following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley 503 Sons, Inc. 1997 and Sambrook et al, Molecular Cloning: A Laboratory Manual 2nd Ed., Cold Spring Harbor Laboratory Press (1989). The polynucleotide sequence may also be cloned using homologous recombination techniques including in vitro as well as in vivo recombination. Such procedures and others are deemed to be within the scope of those skilled in the art. The vector may be, for example, in the form of a plasmid, a viral particle, or a phage. Other vectors include chromosomal, nonchromosomal and synthetic polynucleotide sequences, derivatives of SV40; bacterial plasmids, phage DNA, baculovirus, yeast plasmids, vectors derived from combinations of plasmids and bacteriophage DNA, viral DNA such as vaccinia, adenovirus, fowl pox virus and pseudorabies.

Preferred vectors of the invention include, but are not limited to, the plasmid pCLY10_BG (cf. FIG. 6) and the plasmid pMyxZeo_dif (deposited at DSMZ). The plasmid pCLY10_BG contains the six PKS genes difB, difC, difD, difE, difF, and difG and selection markers for propargation in S. cerevisiae and E. coli. The map of the plasmid pCLY10_BG is shown in FIG. 6. The plasmid pMyxZeo_dif contains the six PKS genes difB, difC, difD, difE, difF, and difG, a zeocin resistance cassette, the mx9 integrase gene, and the vanillate promotor/repressor system. The plasmid pMyxZeo_dif allows integration into the genome and controllable expression in myxobacterium Myxococcus xanthus DK1622. The map of the plasmid pMyxZeo_dif is shown in FIG. 7.

The invention also provides host cells, i.e. transformed cells comprising a nucleic acid sequence of the invention, e.g. a sequence encoding one or more polypeptides or all of a NRPS of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells.

Preferred mammalian cells include e.g. Chinese hamster ovary (CHO) cells, COS cells, 293 cells, PerC6 cells, hybridomas, Bowes melanoma or any mouse or any human cell line. Exemplary insect cells include any species of Spodoptera or Drosophila, including Drosophila S2 and Spodoptera Sf-9. Exemplary fungal cells include any species of Aspergillus. Preferred yeast cell include, e. g. a cell from a Candida, Hansenula, Kluyveromyces, Pichia, Saccharomyces, Schizosaccharomyces, or Yarrowia strain, more preferably from Kluyveromyces lactis, S. cerevisiae, Hansenula polymorpha, Yarrowia lipolytica, or Pichia pastoris. According to the invention, the host cell may be a prokaryotic cell. Preferably, the prokaryotic host cell is a bacterial cell. The term “bacterial cell” includes both Gram-negative and Gram-positive as well as archaeal microorganisms. Suitable bacteria may be selected from e.g. Myxobacterium, Escherichia, Anabaena, Caulobacter, Gluconobacter, Rhodobacter, Pseudornonas, Paracoccus, Ralstonia, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. Preferably, the bacterial cell is selected from the group consisting of Angiococcus disciformis, Pyxidicoccus fallax, B. subtilis, B. amyloliquefaciens, B. licheniformis, B. puntis, B. megaterium, B. halodurans, B. pumilus, G. oxydans, Caulobacter crescentus CB 15, Methylobacterium extorquens, Rhodobacter sphaeroides, Pseudomonas putida, Paracoccus zeaxanthinifaciens, Paracoccus denitrificans, Ralstonia eutropha, E. coli, C. glutamicum, Staphylococcus carnosus, Streptomyces lividans, Sinorhizobium melioti and Rhizobium radiobacter. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)). The nucleic acids or vectors of the invention may be introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets can be used.

Where appropriate, the engineered host cells can be cultured in conventional nutrient media modified as appropriate for activating promoters, selecting transformants or amplifying the nucleic acids of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof. Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps. The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue. Cell-free translation systems can also be employed to produce a polypeptide of the invention. Cell-free translation systems can use mRNAs transcribed from a DNA construct comprising a promoter operationally linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

Host cells containing the polynucleotides of interest, e.g., nucleic acids of the invention, can be cultured in conventional nutrient media modified as appropriate for activating promoters, selecting transformants or amplifying genes. The culture conditions such as temperature, pH and the like, are those previously used with the host cell selected for expression and will be apparent to the ordinarily skilled artisan. The clones which are identified as having the specified enzyme activity may then be sequenced to identify the polynucleotide sequence encoding a portion or all of a PKS of the invention.

Recombinant DNA can be introduced into the host cell by any means, including, but not limited to, plasmids, cosmids, phages, yeast artificial chromosomes or other vectors that mediate transfer of genetic elements into a host cell. These vectors can include an origin of replication, along with cis-acting control elements that control replication of the vector and the genetic elements carried by the vector. Selectable markers can be present on the vector to aid in the identification of host cells into which genetic elements have been introduced. Means for introducing genetic elements into a host cell (e.g. cloning) are well known to the skilled artisan. Other cloning methods include, but are not limited to, direct integration of the genetic material into the chromosome. This can occur by a variety of means, including cloning the genetic elements described herein on non-replicating plasmids flanked by homologous DNA sequences of the host chromosome; upon transforming said recombinant plasmid into a host the genetic elements can be introduced into the chromosome by DNA recombination. Such recombinant strains can be recovered if the integrating DNA fragments contain a selectable marker, such as antibiotic resistance. Alternatively, the genetic elements can be directly introduced into the chromosome of a host cell without use of a non-replicating plasmid. This can be done by synthetically producing DNA fragments of the genetic elements in accordance to the present invention that also contains homologous DNA sequences of the host chromosome. Again if these synthetic DNA fragments also contain a selectable marker, the genetic elements can be inserted into the host chromosome.

A portion or all of a PKS of the invention may be favorably expressed in any of the above host cells. Thus, the present invention provides a wide variety of host cells comprising one or more of the isolated, synthetic or recombinant nucleic acids and/or PKSs of the present invention. The host cell, when cultured under suitable conditions, can be capable of producing a disciformycin according to formula (I) that it otherwise does not produce, or produces at a lower level, in the absence of a nucleic acid of the invention.

A preferred host cell of the invention is a bacterial cell from Myxococcus xanthus DK1622. Particularly preferred as a host cell is DK1622 attB::dif20, a result of a transformation of M. xanthus DK1622 with plasmid pMyxZeo_dif and subsequent integration of the heterologous plasmid DNA into the genome of M. xanthus DK1622.

The invention also relates to an isolated, synthetic or recombinant polypeptide having an amino acid sequence according to any of SEQ ID NOs. 11 to 19, SEQ ID NOs. 34 to 46, or an amino acid sequence encoded by a nucleic acid of the invention.

Also the following methods for producing a compound of formula (I) lie within the scope of the present invention: chemical synthesis, semisynthesis, and biosynthesis including recombinant techniques. Since the structural complexity of the compounds precludes facile total synthetic access, semisynthetic as well as biotechnological approaches are commonly pursued in pharmaceutical research and development. It is understood that the production of compounds of formula (I) is not limited to the use of the particular organism described herein, which is given for illustrative purpose only. The invention also includes the use of any mutants which are capable of producing a compound of formula (I) including natural mutants as well as artificial mutants, e.g. genetically manipulated mutants and the expression of the gene cluster responsible for biosynthesis in a producer strain or by heterologous expression in host strains.

For instance, a compound of formula (I) can be produced by a method comprising the steps: (a) culturing a host cell comprising at least one nucleic acid or a vector according to the invention; and (b) separating and retaining the compound from the culture broth.

The culturing step can be performed in liquid culture, by growing the respective host cell in media containing one or several different carbon sources, and one or different nitrogen sources. Also salts are essential for growth and production. Suitable carbon sources are different mono-, di-, and polysaccharides like maltose, glucose or carbon from amino acids like peptones. Nitrogen sources are ammonium, nitrate, urea, chitin or nitrogen from amino acids. The following inorganic ions support the growth or are essential in synthetic media: Mg-ions, Ca-ions, Fe-ions, Mn-ions, Zn-ions, K-ions, sulfate-ions, Cl-ions, phosphate-ions. The host cell may be a microorganism, e.g. Angiococcus disciformis strain AngJ1 (DSM 27408) or Pyxidicoccus fallax HKI 727 (DSM 28991). Temperatures for growth and production are between 15° C. to 40° C., preferred temperatures are between 25° C. and 35° C., especially at 30° C. The pH of the culture solution is from 5 to 8, preferably a pH of 7.3 to 7.5.

A compound of formula (I) can also be obtained by chemical synthesis in a number of ways well known to one skilled in the art of organic synthesis using usual chemical reaction and synthesis methods, e.g. by the synthesis provided in Example 5 below.

### BRIEF DESCRIPTION OF THE FIGURES

FIG. 1. (A) Biosynthesis gene cluster. Putative polyketide synthase (PKS) encoding genes are shown in dark grey arrows, whereas genes encoding non-PKS genes are shown as light grey arrows. (B) PKS domain organization is shown together with a biosynthesis proposal. Disciformycin assembly is expected to start at DifG, incorporating a malonate and a methylmalonate unit. Polyketide extension is then expected to continue at modules DifB-DifF. Product release takes place by macrocyclization at the TE-domain of DifF. The hydroxy-group at carbon atom C6, to which the valerate is attached in the final compound, does not result from reduction of a keto group. Instead, this hydroxy-group is expected to result from oxidation by a tailoring enzyme during or after assembly. The putative cytochrome P450 gene difA, located adjacent to the PKS genes is considered the most likely candidate for this tailoring step. Suitable genes for further post-PKS modifications, i.e. acylation and glucosylation, could not be found in the vicinity of the PKS gene cluster with structure of disciformycin. The abbreviations have the following meaning: AcT=acyl transferase; AT=acyl transferase domain; CYP=cytochrome P450; DH=dehydrogenase domain; ER=enoyl reductase domain; GT=glycosyl transferase; KS=ketosynthetase domain; KR=ketoreductase domain; T=thiolation domain; TE=thioesterase domain.

FIG. 2. Structure of disciformycin A (1) with carbon atoms numbered.

FIG. 3. Structure of disciformycin A (1) with selected COSY and HMBC correlations. ¹H,¹H-COSY and ¹H,¹H-TOCSY correlations led to six ¹H-spins systems. These partial structures were subsequently linked by series of ¹H,¹³C-HMBC correlations: correlation of methyl group C-15 (δ_{H} 1.96) to C-1 (δ_{C} 169.1), of methylene C-4 (δ_{H} 4.01, 3.58) and methine C-6 (δ_{H} 5.05) to C-5 (δ_{C} 203.9), of methine C-7 (δ_{H} 4.26) and methyl C-16 (δ_{H} 1.91) to C-8 (δ_{C} 134.9), as well as 11-H (δ_{H} 5.61) and 17-H (δ_{H} 1.65) to C-12 (δ_{C} 134.9) established the carbon skeleton of the aglycon. The deep field shift of 11-H (δ_{H} 5.61) indicated an ester linkage at this position which was verified by a HMBC correlation between 11-H and C-1, establishing the lactone ring closure of the aglycon. The configuration of the methyl-substituted double bonds was derived from ROESY correlations. A strong ROESY correlation between 15-H and 3-H demonstrated a Z configuration for the Δ^{2,3} double bond, while the absence of a ROESY correlation between 16-H (δ_{H} 1.91) and 9-H (δ_{H} 5.32) showed an E configuration for the Δ^{8,9} bond. Eventually, a ROESY correlation between 13-H (δ_{H} 5.41) and 17-H (δ_{H} 1.65) indicated a Δ^{12,13} Z configuration in the side chain of the aglycon. A ¹H,¹³C-HMBC correlation from 6-H to C-1′ (δ_{C} 173.2) proved the ester linkage of 3-methylbutyric acid to C-6, simultaneously explaining the deep field shift of 6-H (δ_{H} 5.01).

Since carbon nuclei in furanose sugars are generally less shielded than in related pyranoses and the anomeric configuration can be differentiated by their chemical shifts, the ¹³C NMR data of 1 are characteristic for an α-arabinofuranose configuration (observed δ_{C} 110.6; 83.8; 79.1; 86.3; 63.0, methyl furanoside δ_{C} 109.3; 81.9; 77.5; 84.9; 62.4).^{[1]} The absolute configuration of the arabinose moiety was determined to be D-(−)-arabinose by GC-MS comparison of the (−)-2-butyl glycoside derivative to authentic standards.^{[2]}

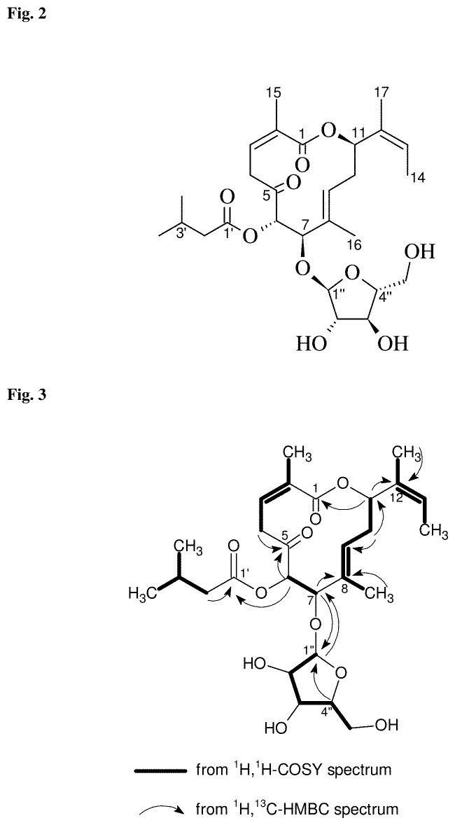

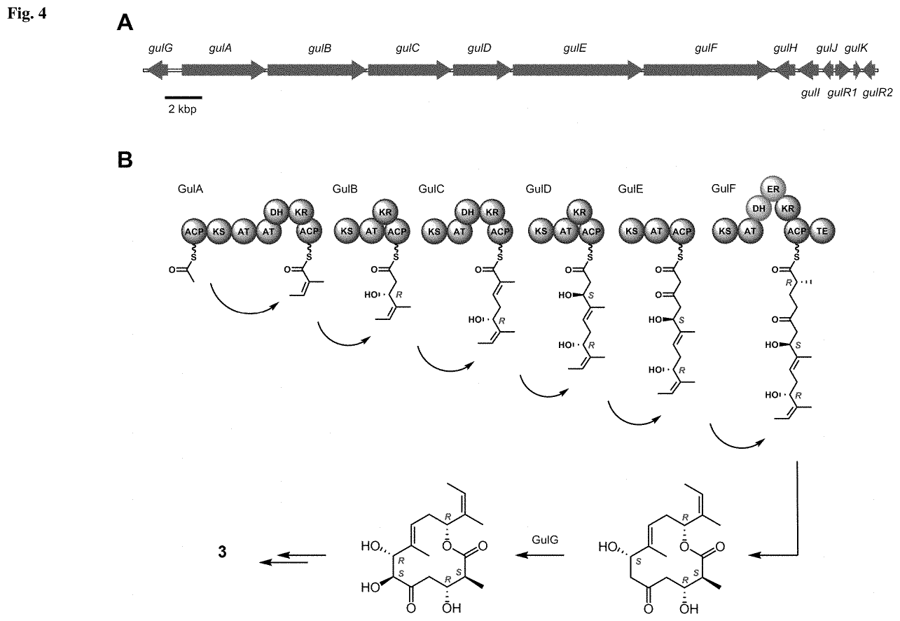

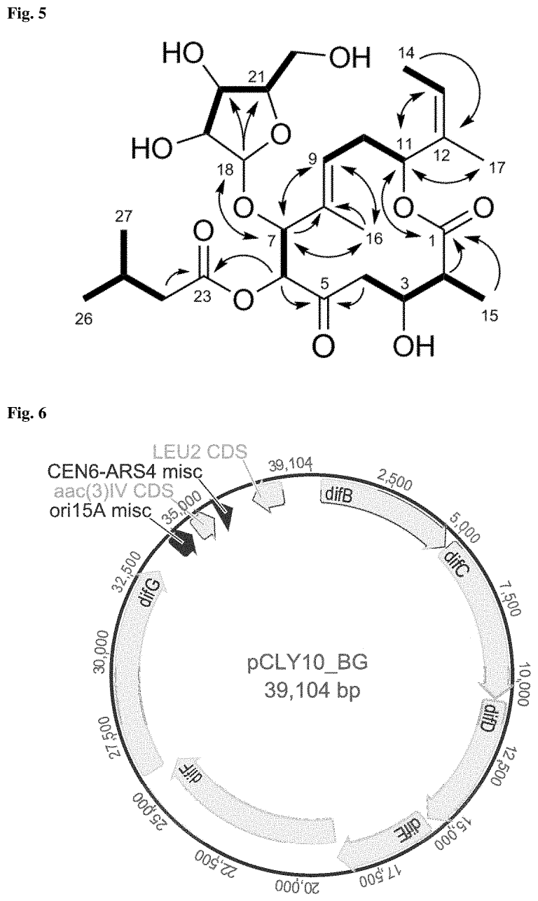

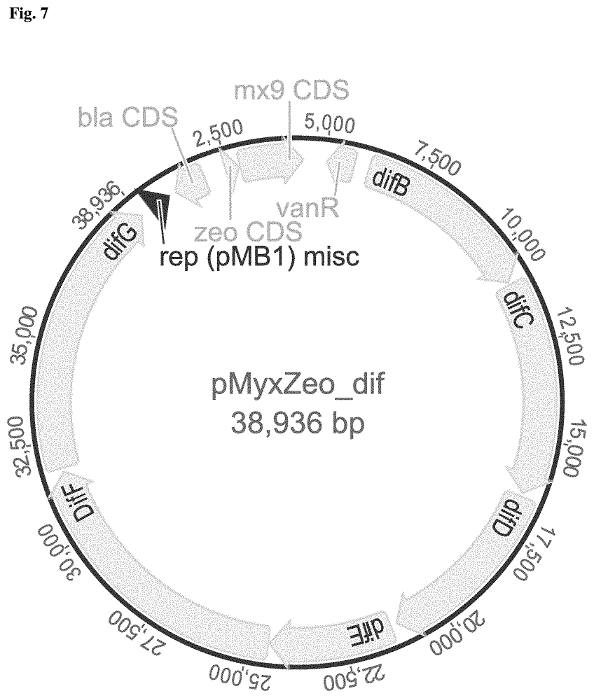

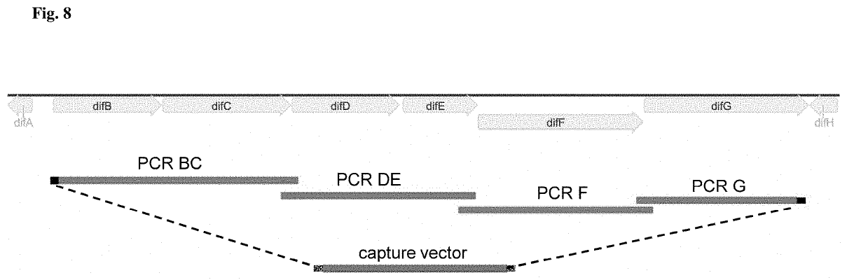

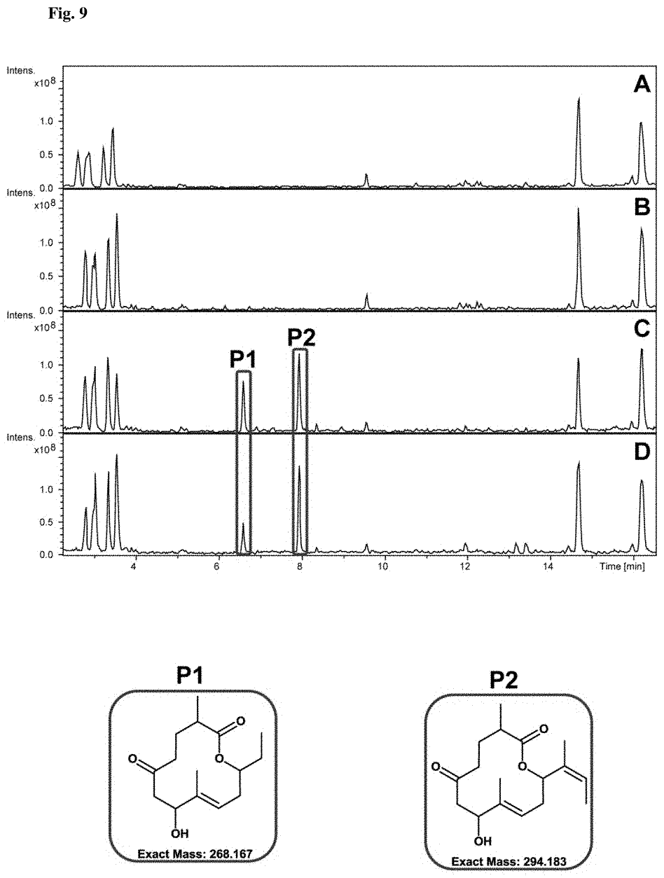

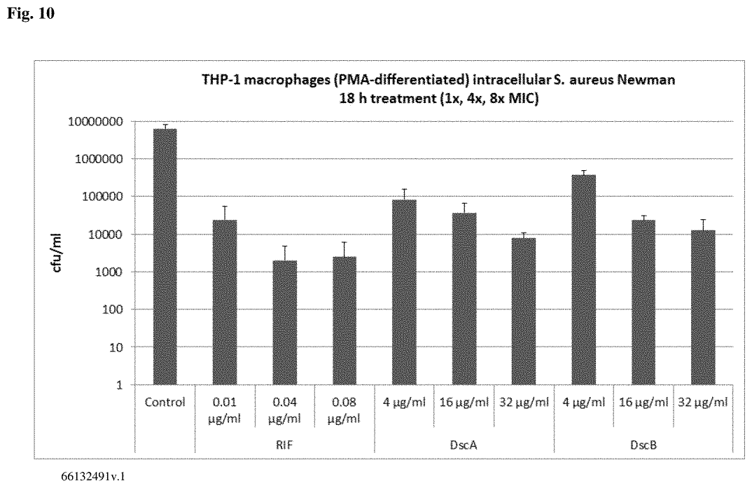

The relative configuration of the aglycon could be derived from vicinal coupling constants and ROESY spectra (FIG. 2). The large coupling constant of 9.5 Hz between 6-H and 7-H and the absence of a ROESY correlation indicated their trans configuration. Strong NOEs were observed for 6-H and 10-Hb with methyl 16-H₃, but not with 11-H. On the other side 7-H shows a strong NOE with 9-H, which itself correlates to 11-H and 10-Ha, indicating a cisoidal relation between 7-H and 11-H.

Having the relative configuration of the aglycon assigned the absolute configuration remained to be established. In this case the usual chemical derivatization was dispensable because the inherent information of the absolute configuration of the d-(−)-arabinosyl residue could be utilized: As a strong ROESY correlation between 1″-H and 7-H confirmed the typical solution conformation of the glycoside. Consequently, a weak but unambiguous correlation between 4″-H and methyl 16-H₃ permitted assigning the configuration of the chiral center C-7 (FIG. 2). Thus the absolute configuration of the disciformycin A aglycon (1) was assigned as (6S,7R,11R).

1D and 2D NMR data of disciformycin B (2) showed that the only difference to 1 was the shifting of the Δ^{2,3} double bond to position Δ^{3,4} with E configuration as indicated by the large coupling constant (J_{3,4}=15.3 Hz). Coupling constants and ROESY correlations of the C-6 to C-11 part remained largely unchanged compared to 1. Therefore, a (6S,7R,11R) configuration can be concluded for 2 as well. To determine the configuration of C-2 a structure model of compound 2 was calculated by MM+ with HyperChem. Due to the rigid structure parts, i.e. the double bond, the keto and the α,β-unsaturated ester, the core part of 2 is locked in a twisted-like configuration, with protons 4-H, 6-H and methyl group 16-H₃ pointing above the main plain. Strong ROESY correlations between 4-H and 2-H on the one and from 3-H to methyl group 15-H₃ on the other hand indicate a 2S configuration.

FIG. 4. (A) Organization of the gul biosynthetic gene cluster. (B) Molecular assembly line deduced from gulA-gulF and proposed biosynthesis of 3. Domain notation: ACP, acyl carrier protein; KS, β-ketoacyl synthase; AT, acyl transferase; DH, dehydrogenase; KR, ketoreductase; ER, enoyl reductase; TE, thioesterase. Note that the DH and ER domain of GulF are skipped for the formation of 3, while they are used for the assembly of 4.

FIG. 5. Structure of gulmirecin A (3) with carbon atoms numbered including COSY (bold lines) and selected HMBC (arrows) correlations.

The identified carbonyl signals in the ¹³C NMR spectrum could be assigned to a ketone (C-5) as well as to two ester functions (C-1, C-23). Aside from the carbonyl groups, four additional carbon atoms (C-8, C-9, C-12, C-13) are sp²-hybridized according to their chemical shifts (cf. Table 5 in Example 6 below). Thus, 3 must feature two carbon-carbon double bonds and two ring structures in order to comply with the required degrees of unsaturation. The signals in the ¹H NMR spectrum were attributed to their directly attached carbon atoms by heteronuclear single-quantum coherence (HSQC). Proton-proton correlation spectroscopy (COSY) revealed six discrete spin systems, all of which could be connected through heteronuclear multiple bond correlation (HMBC) spectroscopy (FIG. 5). The elucidation of the 12-membered lactone ring started from the spin system including CH₃-15 and CH-2 to CH₂-4. The ¹H and ¹³C chemical shifts of CH-3 indicated a secondary alcohol function. Heteronuclear long-range correlations of H₃-15 established the carboxylate group (C-1) at CH-2. Likewise, the placement of the ketone function next to CH₂-4 could be inferred from HMBC interactions between C-5 and the protons at C-3 and C-4. A correlation between H-11 and C-1 in the HMBC spectrum, together with the chemical shift of C-11, unveiled the ester linkage. The resulting partial structure could be expanded with another COSY-derived fragment, which covered CH-9 and CH₂-10 in addition to CH-11. As evidenced by homonuclear and heteronuclear correlations, the olefinic CH-9 must be located adjacent to the quaternary C-8, which possesses a resonance at 133.1 ppm. The two remaining neighboring groups at C-8 were identified as CH₃-16 and CH-7 on the basis of HMBC data. The proton of the methine in position 7 exhibits a ³J coupling to H-6. Except for H-3 and H-4a, H-6 is the only proton correlating with C-5 in the HMBC spectrum, thereby allowing a closure of the macrolide ring. The three substituents at C-6, C-7 and C-11 were identified as an isovaleryl, a furanose, and a 2-but-2-en-yl residue. These moieties were connected to the macrolide on the basis of ¹H,¹³C long-range interactions from H-6 to C-23, from H-7 to C-18, and from H₂-10 to C-12 to give the full planar structure of 3.

The configuration at C-3 was determined as R by preparation of the diastereomeric α-methoxy-α-trifluoromethylphenylacetic acid (MTPA) ester derivatives, removal of the furanose with In(OTf)₃, and calculation of the Δδ^{RS} values. Subsequently, the stereochemistry of the chiral centers C-2, C-6, C-7, and C-11 was concluded from a 2D NOESY experiment. Since no NOE correlation was detected between H-2 and H-3, the two protons must be anti-oriented, i.e. they are placed on opposite sides of the macrolide ring. Together with the preceding Mosher analysis, a 2S configuration could hence be deduced. In the NOESY spectrum, H-3 exhibits crosspeaks with both hydrogen atoms at C-4, suggesting proton-proton torsion angles of about 60° and a staggered conformation, which is also in good agreement with the respective ³J_{HH} values (Table 5). It is evident that the spatial orientations of H-4a and H-4b cannot be derived from their NOE interactions with H-3. However, H-4b also shows an exclusive NOE correlation with H-7, which is only possible when H-4b occupies an axial position and H-7 is located on the same side of the macrolide ring as H-3. The lack of a NOE correlation between H-6 and H-7 hence established the 6S, 7R configuration. Diagnostic NOEs were observed from H-9 to H-7, H-10b and H-11. The latter proton correlates with H-10b, but not with H-10a, which itself shows crosspeaks with its geminal partner and H₃-16. Taken together, H-10b must have an axial orientation and H-11 should be found on the same side of the molecule as H-3 and H-7, indicating an 11R configuration. The NOE-derived 2S, 3R, 6S, 7R, 11R stereochemistry of the macrolide core was confirmed using a biosynthetic approach that has proven reliable for the configurational assignment of oxygen-bearing stereogenic centers in macrolides and other polyketide natural products.

To resolve the stereochemistry of the furanose moiety in 3, the sugar was cleaved from the macrolide core by treatment with In(OTf)₃, followed by a derivatization with 2,4-dihydro-5-methyl-2-phenyl-3H-pyrazol-3-one (PMP). Co-chromatography against the PMP derivatives of commercially available pentoses indicated 3 to contain either arabinose or xylose. Since a chromatographic separation of the corresponding PMP derivatives could not be achieved, the released sugar was independently subjected to reductive amination with anthranilic acid. The product was compared to accordingly prepared standards and, in this way, the sugar residue of 3 was identified as arabinose. A subsequent conversion of the unmodified sugar into a thiacarbamoyl-thiazolidine derivative and chromatographic analysis revealed D-(−)-arabinose as the enantiomer present in 3. The α configuration of the arabinofuranoside was concluded from its ¹³C chemical shifts, and established the S configuration of the anomeric carbon C-18. In conclusion, the absolute stereochemistry of 3 was established as depicted above.

An inspection of the NMR spectra of gulmirecin B (4) revealed the structural relatedness to 3. The missing double bond equivalent (in comparison to 3) could be readily attributed to the absence of the isovalerate moiety at C-6 and the corresponding carbonyl function. Furthermore, the secondary alcohol at C-3 was replaced by a fully reduced methylene function (δ_{H} 1.66 ppm; δ_{C} 28.0 ppm) in 4. This suggests the dehydratase (DH) and enoyl reductase (ER) domain of GulF to be operational (FIG. 4), and lends additional support to the involvement of the annotated gene cluster in gulmirecin biosynthesis. The proposed stereochemistry of 4 was established on the basis of NOE correlations and biosynthetic reasoning. Upon irradiation at the resonance frequency of H-4b, NOE's were observed with H₂-3, H-4a, H-7 and H₃-15. The latter correlation is only possible, when the methyl group at C-2 resides on the same side of the macrolide ring as H-7.

FIG. 6. Plasmid map of the plasmid vector pCLY10_BG containing the core PKS genes of the disciformycin PKS gene cluster, i.e. difB, difC, difD, difE, difF, and difG, and homology arms and selection markers for propagation in S. cerevisiae and E. coli.

FIG. 7. Plasmid map of pMyxZeo_dif containing the core PKS genes of the disciformycin PKS gene cluster, i.e. difB, difC, difD, difE, difF, and difG, driven by the vanillate-inducible Pvan promotor. This recombinant plasmid can integrate into the genome of M. xanthus DK1622. Induction of difBCDEFG expression leads to production of compounds P1 and P2.

FIG. 8. Assembly of the dif-PKS gene cluster by TAR. The six PKS genes difB-difG were amplified as 4 over-lapping PCR products. In parallel, a part of the pCLY10 plasmid was amplified by PCR, carrying homologous sequences for assembly at each end.

FIG. 9. Total ion chromatograms of four cultivations of M. xanthus DK1622 mutants containing inducible heterologous gene cluster. A: mutant with dif-cluster, not induced. B: Mutant control with different cluster, induced. C and D: mutants with dif-cluster, induced with KVan. Mass peaks P1 and P2 are only observed in extracts of the induced mutants with the dif-cluster. Their mass and chemical structure is shown below the chromatograms. The chemical structures of P1 and P2 were elucidated using NMR.

FIG. 10. Intracellular activity of disciformycins A (DscA) and B (DscB) at various concentrations compared to rifampicin (RIF).

The present invention is now further illustrated by the following examples from which further features, embodiments and advantages of the present invention may be taken.

### EXAMPLES

The compounds of the present invention can be prepared in a number of ways well known to one skilled in the art of organic synthesis. The compounds of the present invention can be synthesized using methods known in the art of synthetic organic chemistry or by biotechnological approaches such as fermentation as appreciated by those skilled in the art.

General Experimental Procedures:

Optical rotations were determined with a Perkin-Elmer 241 or a JASCO P-1020 polarimeter. UV spectra were recorded with a Shimadzu UV-Vis spectrophotometer UV-2450 or a Varian UV/Visible Cary spectrophotometer. IR spectra were recorded on a Bruker FT-IR (IFS 55) spectrometer. NMR spectra were recorded with Bruker AM 300 (¹H 300 MHz, ¹³C 75 MHz), Avance III 500 (¹H 500 MHz, ¹³C 125 MHz), ARX 600 (H 600 MHz, ¹³C 150 MHz) and Bruker Ascend 700 (with a 5 mm TXI cryoprobe (¹H 700 MHz, ¹³C 175 MHz)) spectrometers. HRESIMS mass spectra were obtained with an Exactive Mass Spectrometer (Thermo-Scientific) or an Agilent 1200 series HPLC-UV system combined with an ESI-TOF-MS (Maxis, Bruker) [column 2.1×50 mm, 1.7 μm, C₁₈ Acquity UPLC BEH (Waters), solvent A: H₂O+0.1% formic acid; solvent B: AcCN+0.1% formic acid, gradient: 5% B for 0.5 min increasing to 100% B in 19.5 min, maintaining 100% B for 5 min, F_{R}=0.6 mLmin⁻¹, UV detection 200-600 nm].

### Example 1 Biosynthesis and Biophysical Analysis of Disciformycin A and B

Cultivation A (10 L Scale):

Angiococcus disciformis AngJ1 was cultivated in 10 L of medium E (per liter: skimmed milk 4 g; soy meal 4 g; yeast extract 2 g; starch 10 g; MgSO₄×7 H₂O 1 g; Fe-EDTA 8 mg, glycerol 5 g; behenyl alcohol 35 mg) in a biofermenter b10 (Aktiengesellschaft für Biotechnologische Verfahrenstechnik, Schweiz) at 30° C. for 216 h. The pH was regulated with potassium hydroxide (2.5%) and sulfuric acid between 7.3 and 7.5. The stirrer speed was 100-400 rpm, aerated with 0.05 vvm compressed air. The dissolved oxygen content within the fermentation broth was regulated by the stirrer speed to pO₂ 20%. Fermentation was carried out with addition of 1% adsorber resin XAD-16.

Purification A:

For isolation of the active metabolites, XAD-16 was harvested by centrifugation; cells were separated from XAD-16 by flotation and discarded. The XAD was eluted with 750 mL acetone and 750 mL methanol, separately. Bioassays against S. aureus showed that the antibacterial activity was concentrated in the acetone extract. The organic solvent of the acetone fraction was evaporated in vacuo until mostly water remained. This was extracted twice with ethylacetate. The ethylacetate was removed in vacuo; the material was dissolved in 85% aqueous methanol and extracted twice with heptane; subsequently the lower methanol phase was adjusted to 70% methanol and extracted twice with dichloromethane. Bioassays revealing the water fraction contained the main antibacterial activity, antibiotic metabolite 1 was isolated in a bioassay guided fractionation strategy. The extract was fractionated by RP MPLC (column 480×30 mm, ODS/AQ C18 (Kronlab), gradient 37% to 100% methanol in 60 min, flow 30 mL/min, UV peak detection at 210 nm). Active fractions were combined and further fractionated by preparative RP HPLC column 250×21 mm, VP Nucleodur C18 Gravity 5 am, gradient 37% to 100% methanol in 25 min, 50 mM sodium acetate, flow 20 mL/min). A final step of preparative RP HPLC (column 250×21 mm, VP Nucleodur C18 Gravity 5 lam, gradient 28% to 55% methanol in 25 min, 0.5% acetic acid, flow 20 mL/min) provided 1.0 mg of disciformycin A (1).

Cultivation B (70 L Scale):

A. disciformis AngJ1 was cultivated in 70 L of medium P (peptone [Marcor] 2 g/L, starch 8 g/L, probion single cell protein 4 g/L, yeast extract 2 g/L, CaCl₂ 1 g/L, MgSO₄ 1 g/L, Fe-EDTA 8 mg/L, pH 7.5) in a biofermenter P150 (Bioengineering AG, Wald CH) at 30° C. for 148 h. The fermentation parameters were as described for the 10 L fermentation.

Purification B:

For isolation of disciformycin A (1) and B (2), XAD-16 and wet cell mass were harvested by centrifugation. Combined cells and XAD were washed with 30% methanol (1.5 L), and subsequently extracted with methanol (4 L) and acetone (1 L). Methanol and acetone extracts were combined, evaporated and subjected to a solvent partition. The extract was dissolved in 70% aqueous methanol and extracted twice with dichloromethane. The obtained material (10.0 g) was fractionated on silica gel 100 (0.063-0.200 mm, approximately 1 kg) by a gradient from dichloromethane/methanol 98:2 to 8:2 (98:2, 95:5, 92.5:7.5 90:10, 80:20 500-750 mL each). The bioactive fraction “90:10” (219.2 mg) was subjected to preparative RP HPLC (column 250×21 mm, VP Nucleodur C18 Gravity 5 am, gradient 30% to 55% acetonitrile in 25 min, flow 20 mL/min) and afforded 74.3 mg of material containing 1 and 2. A final step of silica gel HPLC (column 250×21 mm, Nucleosil 100 7 μm, isocratic tert-butylmethylether/heptane 1:3 with methanol 2%, flow 25 mL/min) provided 25.4 mg of 1 and 7.6 mg of 2.

Disciformycin A (1):

colorless, amorphous powder, [α]²⁰_{D}−51 (c 0.1, MeOH); UV (MeOH) λₘₐₓ 229 (sh); IR (KBr) 3431, 2961, 2929, 2874, 1731, 1647, 1455, 1383, 1254, 1193, 1124, 1076, 1006, 873 cm⁻¹; ¹H, ¹³C, COSY, HMBC and ROESY NMR data see Table 1; HRESIMS m/z 547.2510 [M+Na]⁺ (calcd for C₂₇H₄₀O₁₀Na, 547.2514).

TABLE 1
NMR spectroscopic data of disciformycin A (1) in
[D₄]methanol (¹H at 600 MHz, ¹³C at 150 MHz).

| # | ¹³C, mult. | ¹H, mult. | COSY | HMBC | ROESY |
|---|---|---|---|---|---|
| 1 | 169.1, C | | | | |
| 2 | 134.5, C | | | | |
| 3 | 130.0, CH | 5.88, ddq (8.1, | 4a, 4b | 4, 15 | 15 > 4b |
| | | 7.3, 1.5) | | | |
| 4 | 44.0, CH₂ | 4.01, dd | 3, 4b, 15 | 2, 3, 5 | 4b, 16 |
| | | (18.3, 8.1) | | | |
| | | 3.58, dd | 3, 4a | | 4a |
| | | (18.3, 7.3) | | | |
| 5 | 203.9, C | | | | |
| 6 | 80.8, CH | 5.05, d (9.5) | 7 | 5, 7, 8, 1′ | 16, |
| | | | | | 4a > 4b |
| 7 | 84.2, CH | 4.26, d (9.5) | 6 | 5, 6, 8, 9, 1″ | 9, |
| | | | | | 1″ > 16 |
| 8 | 134.9, C | | | | |
| 9 | 128.8, CH | 5.32, brd (11.0) | 10a, 10b, 16, | 7, 16 | 7, 10b, 11 |
| 10 | 31.8, CH₂ | 2.82, ddd (14.7, | 9, 10b, 11 | 8, 9, 11 | 10b, 16, 17 |
| | | 11.7, 11.0) | 9, 10a, | | 9, 10a, 11 |
| | | 2.13, m | 11, 16 | | |
| 11 | 75.4, CH | 5.58, dd (11.7, | 10a, 10b | 1, 9, 10, 12, | 10b, |
| | | 3.3) | | 13, 17 | 14 > 17 |
| 12 | 134.9, C | | | | |
| 13 | 123.6, CH | 5.41, qq | 14, 17 | 11, 14, 17 | 17 |
| | | (6.6, 1.5) | | | |
| 14 | 13.1, CH₃ | 1.74, dq | 13, 17 | 12, 13 | 11 |
| | | (6.6, 1.5) | | | |
| 15 | 21.2, CH₃ | 1.96, brs | 3, 4a | 1, 2, 3 | 3 |
| 16 | 12.3, CH₃ | 1.91, dd | 9, 10b | 7, 8, 9 | 6, 10a |
| | | (1.5, 1.5) | | | |
| 17 | 18.3, CH₃ | 1.65, qd | 13, 14 | 11, 12, 13 | 13 |
| | | (1.5, 1.5) | | | |
| 1′ | 174.5, C | | | | |
| 2′ | 43.4, CH₂ | 2.30, d (7.3) | 3′ | 1′, 3′, 4′/5′ | 4′/5′ |
| 3′ | 27.0, CH | 2.11, m | 2′, 4′/5′ | 1′, 2′, 4′/5′ | |
| 4′/5′ | 22.7, CH₃ | 1.00/1.01, d | 3′ | 2′, 3′, 4′/5′ | 2′ |
| | | (6.6) | | | |
| 1″ | 110.6, CH | 5.13, brs | 2″ | 7, 3″, 4″ | 7 |
| 2″ | 83.8, CH | 4.06, m | 1″, 3″ | 1″, 3″, 4″ | |
| 3″ | 79.1, CH | 3.88, dd. | 2″, 4″ | 1″, 2″, 5″ | |
| | | (5.9, 2.9) | | | |
| 4″ | 86.3, CH | 3.94, td | 3″, 5a″, 5b″ | 2″, 3″ | |
| | | (5.7, 3.3) | | | |
| 5″ | 63.0, CH₂ | 3.73, dd | 4″, 5b″ | 3″, 4″ | |
| | | (12.0, 3.3) | | | |
| | | 3.64, dd | 4″, 5a″ | 3″, 4″ | |
| | | (12.0, 5.7) | | | |

Disciformycin B (2):

colorless, amorphous powder, [α]_{D}+64 (c 0.4, MeOH); UV (MeOH) λₘₐₓ 221 (sh), 240 (3.67); IR (KBr) 3429, 2961, 2936, 2875, 1738, 1704, 1627, 1455, 1379, 1299, 1178, 1074, 1031, 856 cm⁻¹; H, ¹³C, COSY, HMBC and ROESY NMR data see Table 2; HRESIMS m/z 547.2516 [M+Na]⁺ (calcd for C₂₇H₄₀O₁₀Na, 547.2514).

TABLE 2
NMR spectroscopic data of disciformycin B (2) in CHCl₃-d
(¹H at 700 MHz, ¹³C at 175 MHz).

| # | ¹³C, mult. | ¹H, mult. | COSY | HMBC | ROESY |
|---|---|---|---|---|---|
| 1 | 171.5, C | | | | |
| 2 | 43.0, CH | 3.35, dqd (9.3, 6.6, 1.1) | 3, 15 | 3, 4, 15 | 4, 15 |
| 3 | 145.9, CH | 6.60, dd (15.3, 9.3) | 2, 4 | 2, 4, 5, 15 | 15 >> 2 |
| 4 | 129.9, CH | 6.37, dd (15.3, 1.1) | 3 | 2, 3, 5 | 2, 6, 16 |
| 5 | 192.1, C | | | | |
| 6 | 78.3, CH | 5.32, d (10.3) | 7 | 5, 7, 8, 1′ | 4, 16 |
| 7 | 81.1, CH | 4.09, d (10.3) | 6 | 5, 6, 8, 9, 16, 1″ | 9, 1″ |
| 8 | 133.3, C | | | | |
| 9 | 129.4, CH | 5.42, m | 10a, 10b | 8, 11 | 7, 10b |
| 10 | 32.1, CH₂ | 2.87, ddd (14.6, 11.7, 11.0) | 9, 10b | 8, 9, 11 | 10b, 16, 17 |
| | | 2.04, m | 9, 10a | 8, 9 | 9, 10a |
| 11 | 72.8, CH | 5.33, dd (11.7, 2.8) | 10a, 10b | 1, 9, 10, 12, | 10b, 14 |
| | | | | 13, 17 | |
| 12 | 133.0, C | | | | |
| 13 | 123.4, CH | 5.40, m | 14 | 11, 12, 14, 17 | 14, 17 |
| 14 | 13.0, CH₃ | 1.71, dq (6.9, 1.3) | 13 | 12, 13 | 11 |
| 15 | 14.1, CH₃ | 1.27, d (6.6) | 2 | 1, 2, 3 | 2, 3 |
| 16 | 12.5, CH₃ | 1.91, brs | 9 | 7, 8, 9 | 6, 10a |
| 17 | 18.0, CH₃ | 1.69, dq (1.5, 1.3) | | 11, 12, 13 | 10a, 13 |
| 1′ | 172.5, C | | | | |
| 2′ | 42.8, CH₂ | 2.36, dd (14.8, 7.3) | 3′ | 1′, 3′, 4′/5′ | 4′/5′ |
| | | 2.31, dd (14.8, 7.3) | | | |
| 3′ | 25.8, CH | 2.16, tsept. (7.3, 6.8) | 2′, 4′/5′ | 1′, 2′, 4′/5′ | 4′/5′ |
| 4′/5′ | 22.4, CH₃ | 1.02, d (6.8) | 3′ | 2′, 3′, 4′/5′ | 2′, 3′ |
| 1″ | 108.2, CH | 5.19, m | 2″ | 7, 3″, 4″ | 7 |
| 2″ | 78.2, CH | 4.03, m | 1″, 3″ | 1″, 3″ | |
| 3″ | 78.3, CH | 4.03, m | 2″, 4″ | 2″, 5″ | |
| 4″ | 88.1, CH | 4.13, m | 3″, 5″a, 5″b | 1″, 3″ | 16, 5″ |
| 5″ | 62.1, CH₂ | 3.89, dd (11.6, 2.6) | 4″, 5″b | 3″ | |
| | | 3.83, dd (11.6, 1.7) | 4″, 5″a | 3″, 4″ | |

### Example 1A Biosynthesis and Biophysical Analysis of Disciformycin C and D

Cultivation was performed according to cultivation B (70 L scale) in Example 1 above.

For isolation of the active metabolites, purification B as described in Example 1 above was conducted, except that the final step of silica gel HPLC was replaced by a further RP HPLC step (column 250×21 mm, VP Nucleodur C18 Gravity 5 am, gradient 30% to 55% acetonitrile in 25 min, flow 20 mL/min) where the fraction containing 1 and 2 was further fractionized to thereby obtain 1.0 mg of disciformycin C (5) and 7.4 mg disciformycin D (6) in addition to 1 and 2.

### Structure Elucidation of Disciformycins C (5) and D (6)

A peak at m/z 579.2776 in the HRESIMS spectrum of 5 provided the molecular formula C₂₈H₄₄O₁₁, which implies a formal addition of CH₄O in comparison to disciformycins A (1) and B (2). The NMR spectra (Table 1A) were highly similar to those of 1 and 2. However, the key difference was the shortfall of signals for the C2/C3 respectively C3/C4 double bond. Instead, additional signals of two methines (δ_{H}, δ_{C} 2.51, 47.5; 3.68, 76.7) and one methoxy moiety (δ_{H}, δ_{C} 3.25, 57.5) were observed. ¹H,¹³C HMBC correlations from H₃-15 to C-1/C-2/C-3 and the methoxy signal to C-3 established constitution of 5. The stereochemistry of the new stereo center C-3 was assigned by the strong ¹H,¹H ROESY correlation from H-3 to H₃-15 together with the absence of a ROESY correlation from H-3 to H-2.

The molecular formula of disciformycin D (6) was deduced as C₂₈H₄₄O₁₀S from its [M+Na]⁺ ion peak cluster at m/z 595.2549 in the HRESIMS spectrum. The NMR spectra of 6 were highly similar to those of 5. The key difference was the high field shift of the signals for C-3 (δ_{C} 41.8, δ_{H} 3.22) and 3SMe (δ_{C} 14.4, δ_{H} 2.07). Therefore, disciformycin D (6) was deduced as the 3-thioether derivative of 5.

TABLE 1A
NMR data (¹H 700 MHz, ¹³C 175 MHz) of disciformycin C (5)
and D (6) in CH₃OH-d₄ for 5 and CHCl₃-d for 6.

| | 5 | | 6 | |
|---|---|---|---|---|
| # | ¹³C. mult. | ¹H | ¹³C | ¹H |
| 1 | 175.1, C | | 174.4, C | |
| 2 | 47.5, CH | 2.51, br s | 44.6, CH | 2.52, m |
| 3 | 76.7, CH | 3.68, m | 41.8, CH | 3.22, m |
| 4 | 47.5, CH₂ | 3.02, m | 41.4, CH₂ | 3.03, m |
| 5 | 205.0, C | | 201.3, C | |
| 6 | 80.6, CH | 5.01, m | 78.4, CH | 4.96, d (9.8) |
| 7 | 84.1, CH | 4.15, d (9.5) | 82.3, CH | 4.17, d (9.8) |
| 8 | 135.8, C | | 132.6, C | |
| 9 | 129.0, CH | 5.30, m | 129.8, CH | 5.45, dd |
| | | | | (11.3, 1.8) |
| 10 | 32.9, CH₂ | 2.82, dt (14.3, | 32.1, CH₂ | 2.73, ddd (14.7, |
| | | 11.8) | | 11.8, 11.3) |
| | | 1.97, m | | 2.00, m |
| 11 | 73.1, CH | 5.81, dd | 71.0, CH | 5.91, dd |
| | | (11.8, 1.9) | | (11.8, 2.3) |
| 12 | 124.5, CH | | 123.6, CH | |
| 13 | 135.0, C | 5.38, m | 133.2, C | 5.36, br q (7.0) |
| 14 | 13.3, CH₃ | 1.67, m | 13.0, CH₃ | 1.66, d6 |
| | | | | (7.0, 1.5) |
| 15 | 15.1, CH₃ | 1.14, d (6.9) | 17.3, CH₃ | 1.29, d (6.7) |
| 16 | 12.3, CH₃ | 1.85, s | 11.7, CH₃ | 1.79, s |
| 17 | 18.5, CH₃ | 1.71, br s | 18.4, CH₃ | 1.71, t (1.5) |
| 1′ | 174.5, C | | 172.7, C | |
| 2′ | 43.6, CH₂ | 2.28, d (7.3) | 42.6, CH₂ | 2.27, t (7.3) |
| 3′ | 27.2, CH | 2.09, m | 25.8, CH | 2.11, m |
| 4′/5′ | 22.8, CH₃ | 0.97, t (6.5) | 22.3, CH₃ | 0.97, d (6.7) |
| 1″ | 110.7, CH | 5.09, s | 108.3, CH | 5.15 br s |
| 2″ | 83.9, CH | 4.01, dd (3.0, 1.3) | 84.0, CH | 4.03, m |
| 3″ | 79.3, CH | 3.82, m | 78.1, CH | 4.03, brs |
| 4″ | 86.3, CH | 3.86, td (5.4, 3.2) | 88.0, CH | 4.14, m |
| | 63.1, CH₂ | 3.68, dd | 62.0, CH₂ | 3.88, dd |
| | | (11.8, 3.2) | | (11.8, 2.8) |
| 5 | | 3.59, dd | | 3.81, (11.8, 2.0) |
| | | (11.8, 5.4) | | |
| 3OMe | 57.5, CH₃ | 3.25, s | | |
| 3SMe | | | 14.4, CH₃ | 2.07, s |

### Example 1B Heterologous Expression System and Precursor Compounds

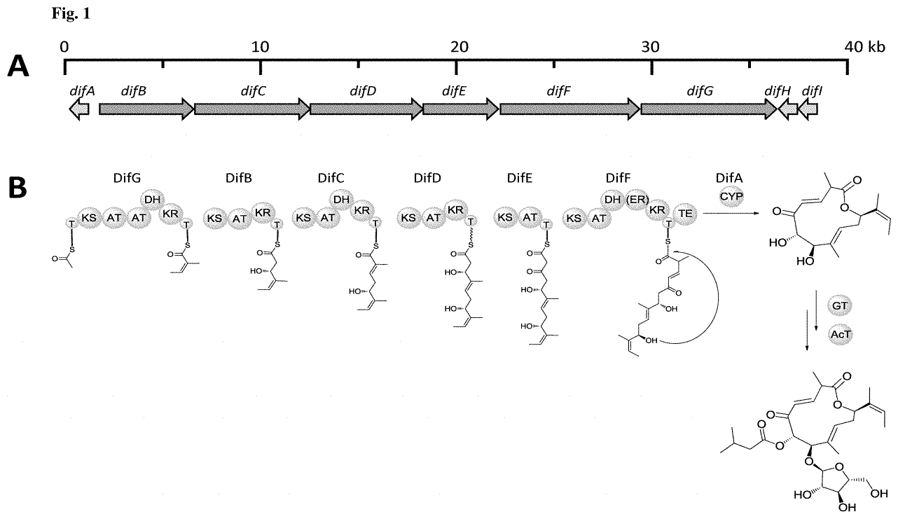

To corroborate the biosynthesis proposal as depicted in FIG. 1 and to enable generation of novel disciformycin derivatives by, e.g., semisynthetic approaches, a heterologous expression system for the production of (a) disciformycin precursor(s), expressing of the PKS genes difB-difG was developed. As producer, the related myxobacterium Myxococcus xanthus DK1622 was chosen, because of its favourable growth characteristics and established genetic tools.

### Cloning of the Dif Cluster by Transformation-Associated Recombination (TAR)

Transformation-associated recombination (TAR) is a cloning strategy that allows linear-to-linear homologous recombination of two or more DNA molecules with suitable homologous sequence overlaps to be assembled as an extrachromosomal plasmid in yeast cells. To select for successfully assembled clones, a genetically modified baker's yeast strain with a selectable mutation is used, S. cerevisiae ATCC 4004247. The strategy for cluster assembly of the PKS genes difB-difG is shown in FIG. 8.

The PKS genes difB-difG were amplified as PCR products that overlap with the neighboring fragment over 30-150 base pairs according to the assembly strategy depicted in FIG. 8. The respective PCR products—PCR BC, PCR DE, PCR F, and PCR G—were generated using genomic DNA from A. disciformis AngJ1 (DSM 27408) as template. The oligonucleotide primers for conducting the PCR reactions for generating the products PCR BC, PCR DE, PCR F, and PCR G were generated using the primer3 software tool [36]. A linear plasmid DNA fragment with homology arms of 40 base pairs was generated using plasmid pCLY10 DNA as template. The homologous overhangs for difB and difG were introduced via the oligonucleotide primers (cf. FIG. 8).

An equimolar mixture of all purified single DNA fragment PCR products was then used for transformation of LEU2-deficient S. cerevisiae ATCC4004247. Clones were selected on leucine-free selection medium. The map of the resulting plasmid pCLY10_BG is shown in FIG. 6.

Modification of the Plasmid for Expression in M. xanthus

The obtained S. cerevisiae clones were screened with colony PCR for correct assembly of the plasmid. Positive clones were then cultivated, their plasmids isolated and transformed into E. coli for plasmid propagation and subsequent restriction analysis. Plasmid DNA from one clone that showed the correct restriction fragments was then transformed into E. coli GB-dir for exchange of the plasmid backbone, which is necessary to allow integration and controllable expression in myxobacterium Myxococcus xanthus DK1622. As a linear capture vector, a PCR product amplified from pMyxZeoPvan plasmid (generated previously by overlap PCR from two plasmids: pMyxoZeo [37], which contains the zeocin resistance cassette and the mx9 integrase gene and pMR3679 [38], which contains the vannilate promotor/repressor system. was generated with primers containing a sequence overhang that is homologous to difB and difG sequences on pCLY10_BG. The plasmid map of the resulting integrative expression plasmid pMyxoZeo_dif is shown in FIG. 7.

Exchange of the plasmid backbone was performed with recombination strain E. coli GB-dir according to standard recombineering protocols [39]. GB-dir clones with recombined plasmid pMyxoZeo_dif were selected by zeocin and ampicillin resistance and verified with colony PCR and restriction analysis. Correct pMyxoZeo_dif plasmids pMyxZeoDif16 and pMyxZeoDif20 were obtained. The sequence of plasmid pMyxZeoDif16 was verified by Illumina sequencing to exclude acquired mutations in the biosynthesis genes.

Heterologous Expression of Precursor Compounds P1 and P2 in M. xanthus

Plasmids pMyxZeoDif16 and pMyxZeoDif20 were transformed into M. xanthus DK1622 following standard protocols. DK1622 clones were selected on CTT Agar with zeocin and genetically verified by PCR. One genetically verified clone of DK1622 attB::dif20 was then grown in triplicates in a 20 ml scale in liquid culture and induced with either 1 mM or 2 mM potassium vanillate (Kvan) when the culture showed visible planctonic growth. For comparison, a non-induced culture and a zeocin-resistant control mutant DK1622 attB::CPN13, carrying another heterologous gene cluster integrated in its genome were cultivated in parallel. Cultivation was continued until the colour of the cultures was turning brown, indicating the dying phase. Cells and growth medium were then extracted twice with ethyl acetate, the extracts dried, redissolved in methanol and subjected to MS-analysis, as shown in FIG. 9. The chromatograms of extracts from M. xanthus DK1622 attB::dif20 cultures (panel A, C and D) and one control clone attB::CPN13 (panel B) were compared. New masses were detected in extracts of induced DK1622 attB2::dif20 cultures (panel C and D), which are absent in the extracts of the non-induced culture and in the induced control culture of attB::CPN13 (panel A and B). The new mass peaks P1 and P2 are marked in boxes in the chromatograms in FIG. 9 and are expected to result from expression of the PKS encoded by the dif-genes. Mass peak P1 has a m/z of [M+H]⁺=269.2 and P2 has a m/z of [M+H]⁺=295.2.

### Structure Elucidation of Products P1 and P2

To obtain further insight into the compound identity, cultivation was scaled up to 400 ml CYE-medium and 800 ml CTT-medium, induced and extracted as before and the putative precursors P1 and P2 were isolated by preparative LC-MS on a Waters Autopurifier (Eschborn, Germany) high pressure gradient system. From the culture in CYE medium, 0.7 mg of each compound P1 and P2 were obtained, whereas from the culture in CTT, 0.3 mg of P1 and 1.0 mg of P2 were obtained. Subsequently, 0.7 mg of each purified compounds P1 and P2 were dissolved in DMSO-d₆ for NMR measurements on a Bruker Ascend 700 spectrometer (data not shown). By comparison of the obtained spectra for P1 and P2 with the previously recorded spectra of disciformycins A and B, the structures of P1 and P2 were elucidated to consist of the disciformycin macrolacton core, lacking the post-PKS modifications and possessing fully saturated C2-C3 and C3-C4-bonds, where the disciformycins A and B have a double bond, respectively. The structure of P1 is identical to P2 except for its side chain, which lacking two carbon atoms C13 and C14. The structures of P1 and P2 are shown in FIG. 9.

### Example 2 Assessment of Antimicrobial Activity of Disciformycin A and B

Bacterial strains. Bacterial wildtype strains used in susceptibility assays were either part of HZI's strain collection or purchased from the German Collection of Microorgansims and Cell Cultures (Deutsche Sammlung von Mikroorganismen und Zellkulturen, DSMZ).

Susceptibility Testing.

Minimum inhibitory concentration (MIC) was determined by microbroth dilution. In brief, overnight cultures of bacteria (EBS medium) were diluted to OD₆₀₀ 0.01. Disciformycin A (1) or B (2) dissolved in DMSO was added directly to the cultures in 96-well plates (Sarstedt, flat bottom) in duplicate and the compound was tested in serial dilution. Methanol was tested as negative control and showed no activity against the test organisms. ^{[a]}Oxytetracyclin hydrochloride, ^{[b]}Gentamycin, and ^{[c]}Vancomycin were tested as positive control. After 16 h incubation at 900 rpm and 30° C. on a microplate shaker (Titramax, Heidolph) absorbance at 600 nm was measured using a microplate reader (POLARstar Omega, BMG Labtech). MIC values were determined by sigmoidal curve fitting. The results are shown in Table 3.

TABLE 3
Antimicrobial activity of disciformycin A (1) and B (2)

| Bacterium | DSM/ATCC | 1 | 2 | Ref.^{[a, b, c]} |
|---|---|---|---|---|
| Gram + | | | | |
| Bacillus subtilis | 10 | 4.2 | 0.83 | 4.2^{[a]} |
| Paenibacillus polymyxa | 36 | 16.6 | 16.6 | 1.7^{[a]} |
| Staphylococcus aureus | 346 | 16.6 | 3.3 | 0.21^{[a]} |
| Staphylococcus aureus Newman | / | 8.0 | 1.2 | / |
| Staphylococcus aureus (MRSA) | 11822 | 4.0 | 0.6 | 0.83^{[c]} |
| Staphylococcus aureus N315 | / | 8.0 | 1.2 | / |
| (MRSA) | | | | |
| Staphylococcus aureus Mu50 | 700699 | 2.0 | 0.6 | / |
| (MRSA/VRSA) | | | | |
| Staphylococcus camosus | 20501 | 7.8 | 2.4 | / |
| Mycobacterium sp. | 43270 | n.i. | n.i. | 0.52^{[a]} |
| Mycobacterium diemhoferi | 43524 | 33.3 | 33.3 | ≤0.052^{[a]} |
| Micrococcus luteus | 20030 | 67 | n.i. | 0.42^{[a]} |
| Nocardioides simplex | 20130 | 33.3 | 16.6 | 3.3^{[a]} |
| Gram − | | | | |
| Pseudomonas aeruginosa | 50071 | n.i. | n.i. | 42.0^{[b]} |
| Chromobacterium violaceum | 30191 | n.i. | n.i. | 0.83^{[a]} |
| Escherichia coli | 1116 | n.i. | n.i. | 0.83^{[a]} |

n.i. = no inhibition up to 67 μg/ml

As demonstrated above, disciformycin A (1) and B (2) have an excellent antimicrobial activity against Gram-positive bacteria, and particularly against staphylococci, such as S. carnosus DSM-20501 (7.8/2.4; MIC in μg/mL for 1 and 2, respectively) and S. aureus Newman^{[5]} (8.0/1.2). In addition, both tested strains of methicillin-resistant S. aureus (MRSA) were inhibited: S. aureus DSM-11822 (4.0/0.6) and S. aureus N315^{[6]} (8.0/1.2). Moreover, these MRSA strains show reduced susceptibility to other antibiotic classes, such as macrolides and quinolones, indicating a putative novel target to be addressed by compounds according to the invention, e.g. 1 and 2, respectively. Notably, the MICs are in the range of reserve antibiotic vancomycin (cf. Table 3). Most importantly, no cross-resistance was observed to classes of antibiotics in use for human therapy, e.g. vancomycin, as demonstrated by the pronounced activity of 1 and 2 against the methicillin- and vancomycin-resistant S. aureus (MRSA/VRSA) Mu50 (ATCC 700699) (2.0/0.6).

### Example 3 Assessment of Antiproliferative Activity of Disciformycin A and B

Cytotoxicity Assay.

Half maximal inhibitory concentrations (IC₅₀) in μg/ml of disciformycin A (1) and disciformycin B (2) for inhibiting the proliferative activity of human HCT-116 colon carcinoma cells, mouse fibroblast cells L929, and Chinese hamster ovary CHO-K1 cells were determined. Cells were seeded at 6×10³ cells per well of 96-well plates (Corning CellBind®) in complete medium (180 μl) and directly treated with disciformycin dissolved in DMSO in serial dilution. The compound was tested in duplicate, as well as the internal solvent control. After 5 d incubation, 5 mg/ml MTT in PBS (20 μL) was added per well and it was further incubated for 2 h at 37° C. The medium was then discarded and cells were washed with PBS (100 μl) before adding 2-propanol/10N HCl (250:1, v/v; 100 μl) in order to dissolve formazan granules. The absorbance at 570 nm was measured using a microplate reader (EL808, Bio-Tek Instruments Inc.). The amount of formazan, i.e. relative absorption, is thereby proportional to the number of actively proliferating cells. The results are shown in Table 4.

TABLE 4
Antiproliferative activity of disciformycin A (1) and disciformycin B (2)

| | IC₅₀ (μg/mL) | |
|---|---|---|
| | 1 | 2 |
| HCT-116 colon carcinoma cells (ACC-581) | 13.0 | >10 |
| Mouse fibroblast cells L929 (ACC-2) | 29 | >10 |
| Chinese hamster ovary CHO-K1 cells (ACC-110) | 16.6 | >10 |

### Example 4 Comparison and Prediction of the Substrate Specificity of the Domains in the Disciformycin PKS Modules

Assembly of the polyketide is expected to start at C14 with incorporation of malonate (mal) and extension by methylmalonate (mmal), mal, mmal, mal, mal, mmal. The polyketide is released as a macrolide, by formation of an ester bond between the carboxyl group at C1 and the hydroxy group at C11. The order of the PKS modules is DifG, DifB, DifC, DifD, DifE, DifF.

At Domains.

Prediction of the substrate specificity of the seven AT domains was done by multiple-alignments of their respective amino acid sequences and comparison of key residues to published data^{[3]}. Specifically, the specificity of the AT domains within the module sequence was predicted based on occupation of critical position (AT fingerprint) according to Mohanty. The ATs of modules DifB, DifF, DifE and AT1 of module DifG have a motif characteristic for malonate specific ATs. The ATs of modules DifC, DifF and AT2 from DifG have a methylmalonate-specific motif. Accordingly, the predicted substrate of the AT-domains from modules DifB, DifD, DifE and DifG1 is malonate, while AT-domains from modules DifC, DifF and DifG2 are predicted to be methylmalonate. This is consistent with the observations in the disciformycin molecule.

Kr Domains.

The polyketide core of disciformycin contains three stereocenters and three double bonds, as can be taken from FIG. 2. The hydroxy group in S-configuration at C6 results from hydroxylation and not from keto-reduction, and is therefore excluded from the analysis. The configuration of hydroxy groups and double bonds that result from KR activity is determined by the respective KR domain that is responsible for reduction of the keto group during metabolite assembly. Stereoselectivity can be predicted from the primary sequence of the KR-domain, since most KRs group into two distinct types, A- and B-type, which accept their substrate in different ways, and thus produce a secondary alcohol of opposite configuration. Briefly, an A-type KR activity results in an L-configured alcohol, while B-type KRs yields D-configured alcohols. Further, a trans-configured double bond results from a dehydrogenase activity on a hydroxy group in a R-configuration, while a cis-double bond results from an S-configured hydroxy group. While B-type KR domains show a conserved “LDD”-motif in the loop region of the enzyme, A-type KRs lack this motif and instead contain a conserved Trp-residue in the catalytic centre.

The stereoselectivity of the KR-domains can be predicted from their primary sequence by multiple alignments and screening for sequence motifs^{[4]}. By multiple alignments of the five KR sequences, loop region and catalytic region were investigated for presence of key residues. The KR of DifB, which reduces the keto group at C11 lacks the typical B-type loop motif and is clearly A-type, because the LDD motif is absent and the critical Trp in the catalytic centre is present. KRs of DifC, DifD and DifF are all B-type, with the conserved LDD-motif being replaced by “LED” in DifD and “LQD” in DifF. The KR sequence of DifG shows neither the conserved LDD-motif nor the conserved Trp in the catalytic region. A prediction of its stereoselectivity is therefore not possible by this method. Observed and predicted stereochemistry agree for the KRs of DifC and DifD. For the Z Δ^{12,13} double bond and the E Δ^{8,9} double bond, the KR domain sequences correspond clearly to A-type KR for DifG and a B-type KR for DifC, respectively. The “R”-configured hydroxyl group at carbon atom C7, to which the arabinose is attached, results from keto-reduction by the KR of DifD. For the DifG KR, no clear prediction is possible, yet the observed cis-configuration of the double bond at this position suggests that the KR must be A-type, producing a hydroxy-group in S-configuration which is converted to a cis-double bond by the DifG DH domain. Reduction by DifB, which is clearly A-type, should lead to an S-configured OH-group at C11, but an R-configured OH-group is observed here. The cis-double bond at C3 (Z Δ^{2,3} double bond) is expected to result from DH activity on an S—OH group, but the corresponding DifF KR is B-type. In these two cases, prediction and observed stereochemistry disagree. A possible explanation might be an isomerization during macrocyclization. Notably, module DifF contains a domain of unknown function which appears to be an inactive ER domain. Sequence analysis by BLAST conserved domain search revealed a NADPH binding motif as well as a quinone reductase motif. This unusual domain may catalyze the isomerization which leads to the observed stereochemistry.

### Example 5 Synthesis of Disciformycin

A compound of formula (I) according to the present invention can be synthesized using the starting materials and route of synthesis described below, together with synthetic methods known in the art of synthetic organic chemistry, or variations thereon as appreciated by those skilled in the art. Preferred methods include, but are not limited to, those methods described below or in the respective references cited. Unless otherwise specified all starting materials and reagents are of standard commercial grade, and are used without further purification, or are readily prepared from such materials by routine methods. Those skilled in the art of organic synthesis will recognize that starting materials and reaction conditions may be varied including additional steps employed to produce compounds encompassed by the present invention.

### (1) Preparation of (Z)-3-iodo-2-methylprop-2-en-1-ol^{[7]}

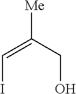

To a solution of CuI (12.8 mg, 67 μmol, 0.1 eq.) in THF (0.7 ml) at −20° C. was added propargyl alcohol (39.7 μl, 672 μmol, 1.0 eq.). A 3 M solution of methylmagnesium bromide in Et₂O (0.49 ml, 1.48 mmol, 2.2 eq.) was added slowly, the temperature was raised to −10° C. and the solution stirred for 30 min. A solution of I₂ (85.3 mg, 672 μmol, 1.0 eq.) in Et₂O/THF (0.3 ml, 1:1) was added and the mixture was allowed to warm to room temperature. The solution was poured into a mixture of NH₄Cl (0.7 ml) and brine (0.7 ml). The aqueous phase was extracted with Et₂O (3×0.4 ml), the combined organic phases were dried (MgSO₄) and concentrated. The residue was purified by column chromatography to furnish the product (100 mg, 504 μmol, 75%) in good yield.

### (2) Preparation of (Z)-1-(((3-iodo-2-methylallyl)oxy)methyl)-4-methoxybenzene^{[8]}

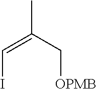

To a suspension of NaH (13.2 mg, 554 μmol 1.1 eq.) in DMF at 0° C. was slowly added a solution of (Z)-3-iodo-2-methylprop-2-en-1-ol (100 mg, 504 μmol, 1.0 eq.) in THF (0.3 ml). The resulting mixture was stirred for 30 min, prior to addition of PMBCl (68.4 μl, 504 μmol, 1.0 eq.) in THF (0.1 ml). After stirring for 2 h, Et₂O and H₂O were added. The phases were separated and the organic phase was washed with H₂O and brine. The dried (MgSO₄) organic layer was concentrated and the residue was purified by column chromatography to give the product (130 mg, 408 μmol, 81%) in good yield.

### Preparation of (S)-4-benzyl-3-(2-(benzyloxy)acetyl)oxazolidin-2-one^{[9]}

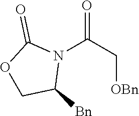 [SMILES: O=C(COCc1ccccc1)N1C(=O)OC[C@@H]1Cc1ccccc1]

To a solution of (S)-4-benzyloxazolidin-2-one (40.0 mg, 224 μmol, 1.0 eq.) in THF (1.1 ml) at −78° C. was added 1.57 M nBuli in hexane (0.14 ml, 224 μmol, 1.0 eq.), followed by addition of benzyloxyactyl chloride (38.8 μl, 246 μmol, 1.1 eq.). After stirring for 1 h at −78° C. the solution was allowed to warm to room temperature over 30 min. The reaction was quenched by addition of NH₄Cl (0.2 ml). The mixture was concentrated to a slurry which was extracted with CH₂Cl₂ (2×0.4 ml). The combined organic phases were washed with 1 M NaOH (0.3 ml) and brine (0.3 ml), dried (Na₂SO₄) and concentrated. The residue was purified by column chromatography to give the product in very good yield (70.6 mg, 217 μmol, 97%).

### (3) Preparation of (3S,4R,5R)-4-((acetyl-12-chloranyl)oxy)-5-(((acetyl-12-chloranyl)oxy)methyl)-2-bromotetrahydrofuran-3-yl 2-chloroacetate^{[10]}

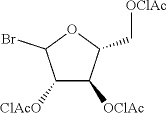

To a solution of D-Arabinose (32.7 mg, 218 μmol, 1.0 eq.) in methanol (0.8 ml) at 0° C. was dropwise added acetlychloride (12.7 μl, 178 μmol, 0.9 eq.). The reaction was stirred for 1 h at room temperature. After addition of pyridine (164 μl, 2.03 mmol, 9.3 eq.) the mixture was concentrated and the residue was coevaporated with CHCl₃.

The residue was dissolved in DMF (0.3 ml) and the solution cooled to 0° C. Na₂CO₃ (104 mg, 981 μmol, 4.5 eq.) was added, followed by chloroacetyl chloride (78.0 μl, 981 μmol, 4.5 eq.) in DMF (0.1 ml). The reaction was stirred overnight at room temperature before being quenched by addition of H₂O. After stirring for 30 min, the solution was extracted with Et₂O (3×0.7 ml). The combined organic layers were dried (MgSO₄) and concentrated. The residue was purified by column chromatography to furnish the product (24.2 mg, 61.1 μmol, 28%).

This compound (24.2 mg, 61.1 μmol, 1.0 eq.) was dissolved in AcOH (0.6 ml) and 45% HBr in AcOH (0.8 ml) was added. The reaction was stirred for 1 h at room temperature. CH₂Cl₂ (0.8 ml) and ice-cold H₂O (0.7 ml) were added and the organic layer was washed with cold H₂O (0.7 ml) and cold saturated NaHCO₃ (2×0.3 ml). The dried organic phase was concentrated to give the product (19.3 mg, 43.4 μmol, 71%), which was used in the following glycosylation without further purification.

### (4) Preparation of (Z)-2-methylbut-2-en-1-ol^{[11]}

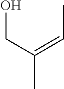

To a suspension of LiAlH₄ (96.0 mg, 2.53 mmol, 2.5 eq.) in Et₂O (1.2 ml) at 0° C. was slowly added a solution of angelica acid methyl ester (0.12 ml, 1.01 mmol, 1.0 eq.) in Et₂O (1.9 ml). The mixture was stirred at room temperature for 1 h before being treated with more LiAlH₄ (30.7 mg, 0.81 mmol, 0.8 eq.) in Et₂O (1.3 ml). The reaction was quenched after 30 min by addition of H₂O (0.3 ml), 15% aqueous NaOH (0.3 ml) and more H₂O (1.0 ml). After filtration the solution was dried (MgSO₄) and concentrated. Kugelrohr distillation afforded the product (65.1 mg, 756 μmol, 75%) as an oil.

### (5) Preparation of (Z)-2-methylbut-2-enal^{[12]}

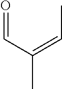

To a solution of (Z)-2-methylbut-2-en-1-ol (65.1 mg, 756 μmol, 1.0 eq.) in CH₂Cl₂ (1.1 ml) was added MnO₂ (1.08 g, 12.5 mmol, 16.5 eq.) and the reaction was stirred for 24 h at room temperature. The solid was filtered off and the solvent removed under reduced pressure. The product (50.9 mg, 605 μmol, 80%) was obtained as a colourless liquid via short path distillation.

### (6) Preparation of (R,Z)-3-hydroxy-1-((S)-4-isopropyl-2-thioxothiazolidin-3-yl)-4-methylhex-4-en-1-one^{[13]}

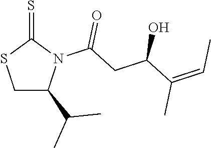 [SMILES: C/C=C(/C)[C@H](O)CC(=O)N1C(=S)SC[C@@H]1C(C)C]

To a solution of N-Acetyl thiazolidinthione (209 mg, 1.03 mmol, 1.7 eq.) in CH₂Cl₂ (4.4 ml) at −40° C. was added TiCl₄ (0.12 ml, 1.09 mmol, 1.8 eq.). After 5 min DIPEA (0.19 ml, 1.09 mmol, 1.8 eq.) was added and the deep red mixture was stirred for 2 h at −40° C. before being cooled to −78° C. (Z)-2-methylbut-2-enal (50.9 mg, 605 μmol, 1.0 eq.) in CH₂Cl₂ (0.6 ml) was added to the reaction. After 10 min the mixture was poured into pH7 phosphate buffer (22 ml) and the aqueous phase was washed with CH₂Cl₂ (3×). The combined organic phases were dried (Na₂SO₄) and concentrated. The product (134 mg, 466 μmol, 77%) was obtained via column chromatography of the residue.

### (7) Preparation of (R,Z)-3-((tert-butyldimethylsilyl)oxy)-1-((S)-4-isopropyl-2-thioxothiazolidin-3-yl)-4-methylhex-4-en-1-one^{[14]}

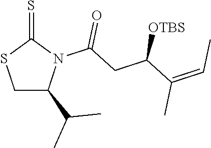 [SMILES: C/C=C(/C)[C@@H](CC(=O)N1C(=S)SC[C@@H]1C(C)C)O[Si](C)(C)C(C)(C)C]

To a stirred solution of (R,Z)-3-hydroxy-1-((S)-4-isopropyl-2-thioxothiazolidin-3-yl)-4-methylhex-4-en-1-one (134 mg, 466 μmol, 1.0 eq.) in CH₂Cl₂ (1.0 ml) was added 2,6-lutidine (92.0 μl, 792 μmol, 1.7 eq.). The mixture was cooled to −78° C. and TBSOTf (214 μl, 932 μmol, 2.0 eq.) was added dropwise. The reaction was stirred at −78° C. for 1 h and at 0° C. for another 1 h. After addition of pH7 phosphate buffer (3.3 ml) and CH₂Cl₂ the phases were separated and the aqueous phase was extracted with CH₂Cl₂ (2×3.3 ml). The combined organic phases were washed with H₂O (3.3 ml), dried and concentrated. The product (185 mg, 461 μmol, 99%) was obtained by column chromatography.

### (8) Preparation of (R,Z)-3-((tert-butyldimethylsilyl)oxy)-4-methylhex-4-enal^{[15]}

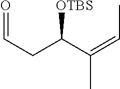 [SMILES: C/C=C(/C)[C@@H](CC=O)O[Si](C)(C)C(C)(C)C]

To a solution of (R,Z)-3-((tert-butyldimethylsilyl)oxy)-1-((S)-4-isopropyl-2-thioxothiazolidin-3-yl)-4-methylhex-4-en-1-one (185 mg, 461 μmol 1.0 eq.) in PhMe (1.1 ml) was added 1 M DIBALH (1.15 ml, 1.15 mmol, 2.5 eq.) at −78° C. The reaction was quenched after 1 h by addition of EtOAc (0.9 ml), followed by addition of saturated Rochelle's salt (2.9 ml). After stirring at room temperature, the phases were separated and the aqueous phase was extracted with EtOAc. The combined organic phases were dried (Na₂SO₄) and concentrated. The residue was purified via column chromatography to furnish the product (105 mg, 433 μmol, 94%) in very good yield.

### (9) Preparation of Ethyl (R,2E,6Z)-5-((tert-butyldimethylsilyl)oxy)-2,6-dimethylocta-2,6-dienoate^{[16]}

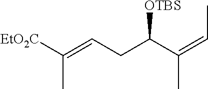 [SMILES: C/C=C(/C)[C@@H](C/C=C(\C)C(=O)OCC)O[Si](C)(C)C(C)(C)C]

To a solution of (R,Z)-3-((tert-butyldimethylsilyl)oxy)-4-methylhex-4-enal (105 mg, 433 μmol, 1.0 eq.) in THF (6.4 ml) was added (carbethoxyethylidene)triphenylphosphorane (471 mg, 1.30 mmol, 3.0 eq.). The mixture was stirred for 24 h at reflux. The crude material was purified by column chromatography to give the product (141 mg, 433 μmol, 100%).

### (10) Preparation of (R,2E,6Z)-5-((tert-butyldimethylsilyl)oxy)-2,6-dimethylocta-2,6-dien-1-ol^{[17]}

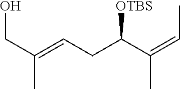 [SMILES: C/C=C(/C)[C@@H](C/C=C(\C)CO)O[Si](C)(C)C(C)(C)C]

A solution of Ethyl (R,2E,6Z)-5-((tert-butyldimethylsilyl)oxy)-2,6-dimethylocta-2,6-dienoate (141 mg, 433 μmol, 1.0 eq) in THF (0.33 ml) was added to a suspension of LiAlH₄ (49.3 mg, 1.30 mmol, 3.0 eq.) in THF (0.08 ml) at 0° C. After stirring for 20 min at room temperature H₂O (0.05 ml) was added, followed by 2M NaOH (0.02 ml). The mixture was filtered through celite and was washed with brine, dried (MgSO₄) and concentrated to give the product (118 mg, 416 μmol, 96%).

### (11) Preparation of (R,2E,6Z)-5-((tert-butyldimethylsilyl)oxy)-2,6-dimethylocta-2,6-dienal^{[18]}

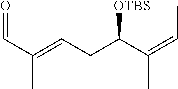 [SMILES: C/C=C(/C)[C@@H](C/C=C(\C)C=O)O[Si](C)(C)C(C)(C)C]

To a solution of (R,2E,6Z)-5-((tert-butyldimethylsilyl)oxy)-2,6-dimethylocta-2,6-dien-1-ol (118 mg, 416 μmol, 1.0 eq.) in Et₂O (2.0 ml) was added MnO₂ (542 mg, 6.24 mmol, 15.0 eq.) and the mixture was stirred for 2 h. The reaction was filtered over a silica gel column, eluting with EtOAc (16 ml). The resulting solution was concentrated to give the product (110 mg, 391 μmol, 94%).

### (12) Preparation of (S)-4-benzyl-3-((2S,3R,4E,7R,8Z)-2-(benzyloxy)-7-((tert-butyl-dimethylsilyl)oxy)-3-hydroxy-4,8-dimethyldeca-4,8-dienoyl)oxazolidin-2-one^{[19]}

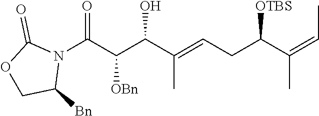 [SMILES: C/C=C(/C)[C@@H](C/C=C(\C)[C@@H](O)[C@H](OCc1ccccc1)C(=O)N1C(=O)OC[C@@H]1Cc1ccccc1)O[Si](C)(C)C(C)(C)C]

To a solution of (S)-4-benzyl-3-(2-(benzyloxy)acetyl)oxazolidin-2-one (70.6 mg, 217 μmol, 1.0 eq.) in PhMe (0.3 ml) was added Et₃N (39.1 μl, 282 μmol, 1.3 eq.). The mixture was cooled to −50° C., followed by careful addition of Bu₂BOTf (77.4 μl, 239 μmol, 1.1 eq.). The reaction was stirred for 1.5 h at −50° C. A solution of (R,2E,6Z)-5-((tert-butyldimethyl-silyl)oxy)-2,6-dimethylocta-2,6-dienal (110 mg, 391 μmol, 1.8 eq.) in PhMe (0.3 ml) was added to the mixture via transfer cannula. The reaction was warmed to −30° C. and stirred for 1.5 h. The reaction was quenched by addition of MeOH (0.2 ml), pH7 buffer (0.2 ml), and H₂O₂ (0.2 ml) and was stirred for 1 h. The organic phase was separated and the aqueous phase was washed with Et₂O (3×15 ml). The combined organic phases were dried (MgSO₄) and concentrated. The product (102 mg, 167 μmol, 77%) was purified using column chromatography.

### (13) Preparation of (S)-4-benzyl-3-((2S,3R,4E,7R,8Z)-2-(benzyloxy)-7-((tert-butyl-dimethylsilyl)oxy)-3-((tert-butyldiphenylsilyl)oxy)-4,8-dimethyldeca-4,8-dienoyl) oxazolidin-2-one ^{[20]}

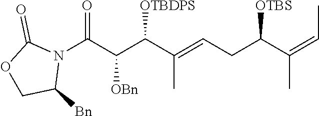

To a solution of (S)-4-benzyl-3-((2S,3R,4E,7R,8Z)-2-(benzyloxy)-7-((tert-butyldimethylsilyl)oxy)-3-hydroxy-4,8-dimethyldeca-4,8-dienoyl)oxazolidin-2-one (102 mg, 167 μmol, 1.0 eq.) in DMF (1.0 ml) at 0° C. was added imidazole (56.8 mg, 835 μmol, 5.0 eq.). A solution of TBDPSCl (0.13 ml, 501 μmol, 3.0 eq.) in DMF (0.2 ml) was added dropwise and the reaction was allowed to warm to room temperature overnight before being quenched by slow addition of saturated NH₄Cl. Et₂O was added and the phases were separated. The aqueous layer was extracted with Et₂O (4×). The combined organic phases were washed with saturated NH₄Cl (2×) and H₂O (2×), dried (MgSO₄) and concentrated. The product (139 mg, 164 μmol, 98%) was obtained via column chromatography of the residue.

### (14) Preparation of (2R,3R,4E,7R,8Z)-2-(benzyloxy)-7-((tert-butyldimethylsilyl) oxy)-3-((tert-butyldiphenylsilyl)oxy)-4,8-dimethyldeca-4,8-dien-1-ol^{[21]}

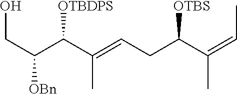

To a solution of (S)-4-benzyl-3-((2S,3R,4E,7R,8Z)-2-(benzyloxy)-7-((tert-butyldimethylsilyl)oxy)-3-((tert-butyldiphenylsilyl)oxy)-4,8-dimethyldeca-4,8-dienoyl)oxazolidin-2-one (139 mg, 164 μmol, 1.0 eq.) in THF (0.8 ml) and H₂O (4.3 μl, 238 μmol, 1.5 eq.) at 0° C. was added a 0.65 M solution of lithium borohydride in THF (0.33 ml, 213 μmol, 1.3 eq.). After 1 h the reaction was quenched by addition of saturated NH₄Cl (0.8 ml). The aqueous phase was extracted with EtOAc (2×1.0 ml). The combined organic phases were washed with brine (0.6 ml), dried (Na₂SO₄) and concentrated. The residue was purified via column chromatography to give the pure product (92.9 mg, 138 μmol, 84%).

### (15) (2S,3R,4E,7R,8Z)-2-(benzyloxy)-7-((tert-butyldimethylsilyl)oxy)-3-((tert-butyl diphenylsilyl)oxy)-4,8-dimethyldeca-4,8-dienal^{[22]}

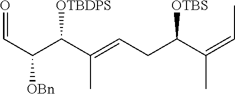

A solution of DMSO (21.6 μl, 304 μmol, 2.2 eq.) in CH₂Cl₂ (0.1 ml) was added to a solution of oxalyl chloride (13.0 μl, 152 μmol, 1.1 eq.) in CH₂Cl₂ (0.2 ml) at −60° C. After 5 min (2R,3R,4E,7R,8Z)-2-(benzyloxy)-7-((tert-butyldimethylsilyl)oxy)-3-((tert-butyl-diphenylsilyl)oxy)-4,8-dimethyldeca-4,8-dien-1-ol (92.9 mg, 138 μmol, 1.0 eq.) in CH₂Cl₂ (0.2 ml) was added to the mixture and the solution was stirred at −60° C. for 15 min. NEt₃ (95.9 μl 692 μmol, 5.0 eq.) was added and the mixture was stirred for 5 min at −60° C. before being warmed to room temperature. The reaction was quenched by addition of H₂O (0.3 ml), the organic layer was washed with brine (0.3 ml), dried (MgSO₄) and concentrated to give the product (91.9 mg, 137 μmol, 99%).

### (16) Preparation of (5R,9R,E)-5-((1R)-(benzyloxy)(oxiran-2-yl)methyl)-9-((Z)-but-2-en-2-yl)-2,2,6,11,11,12,12-heptamethyl-3,3-diphenyl-4,10-dioxa-3,11-disilatridec-6-ene^{[23]}

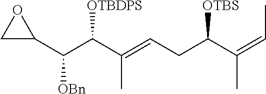

To a solution of Me₃S⁺I⁻ (42.0 mg, 206 μmol, 1.5 eq.) in THF (0.5 ml) was added KHMDS (35.7 mg, 179 μmol, 1.3 eq.). After 1 h, a solution of (2S,3R,4E,7R,8Z)-2-(benzyloxy)-7-((tert-butyldimethylsilyl)oxy)-3-((tert-butyldiphenylsilyl)oxy)-4,8-dimethyldeca-4,8-dienal (91.9 mg, 137 μmol, 1.0 eq.) in THF (0.2 ml) was added via transfer cannula. The reaction was quenched with H₂O (0.04 ml) after 30 min and the solution was concentrated. The obtained residue was redissolved in Et₂O (0.8 ml) and H₂O (0.5 ml) and the aqueous layer was washed with Et₂O (0.3 ml). The combined organic phases were washed with H₂O (0.3 ml) and brine (0.3 ml), dried (MgSO₄) and concentrated. The residue was purified by column chromatography, yielding the product (93.2 mg, 136 μmol, 99%) in very good yield.

### (17) Preparation of (2Z,6R,7R,8E,11R,12Z)-6-(benzyloxy)-11-((tert-butyldimethyl silyl)oxy)-7-((tert-butyldiphenylsilyl)oxy)-1-((4-methoxybenzyl)oxy)-2,8,12-trimethyltetradeca-2,8,12-trien-5-ol^{[24]}

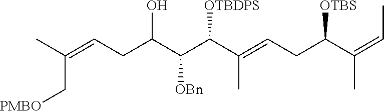

To a dry flask containing Mg (9.92 mg, 408 μmol, 3.0 eq.) in THF (0.1 ml), (Z)-1-(((3-iodo-2-methylallyl)oxy)methyl)-4-methoxybenzene (13.0 mg, 40.8 μmol, 0.3 eq.) was added. After 30 s THF (3 ml) was added and the reaction cooled to 0° C. Additional (Z)-1-(((3-iodo-2-methylallyl)oxy)methyl)-4-methoxybenzene (117 mg, 367 μmol, 2.7 eq.) was added. The solution was stirred for further 2 h and then stirring of the reaction was stopped. After 3 h the mixture was added via transfer cannula to a 0.1 M solution of LiCuCl₄ (0.10 ml, 10.2 μmol, 0.1 eq.) in THF (0.6 ml) at −35° C. The reaction was stirred for 35 min, then (5R,9R,E)-5-((1R)-(benzyloxy)(oxiran-2-yl)methyl)-9-((Z)-but-2-en-2-yl)-2,2,6,11,11,12,12-heptamethyl-3,3-diphenyl-4,10-dioxa-3,11-disilatridec-6-ene (93.2 mg, 136 μmol, 1.0 eq.) in THF (0.6 ml) was added via transfer cannula. After 10 min the reaction was quenched by addition of saturated NH₄Cl (0.2 ml), together with addition of Et₂O (0.5 ml). The mixture was warmed to 10° C. under vigorous stirring. Washing of the organic phase with brine multiple times was followed by extraction of the combined aqueous phases with Et₂O (1×). The combined organic phases were dried (MgSO₄) and concentrated. The residue was purified by column chromatography to furnish the product (115 mg, 132 μmol, 97%) in very good yield.

### (18) Preparation of (6R,7R,11R,E)-6-(benzyloxy)-11-((Z)-but-2-en-2-yl)-7-((tert-butyldiphenylsilyl)oxy)-5-((Z)-4-((4-methoxybenzyl)oxy)-3-methylbut-2-en-1-yl)-8,13,13,14,14-pentamethyl-4,12-dioxa-2-thia-13-silapentadec-8-ene^{[25]}

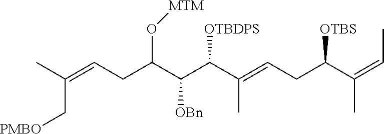

A stirred suspension of NaH (5.7 mg, 238 μmol, 1.8 eq.) in THF (0.2 ml) was treated with (2Z,6R,7R,8E,11R,12Z)-6-(benzyloxy)-11-((tert-butyldimethylsilyl)oxy)-7-((tert-butyldiphenylsilyl)oxy)-1-((4-methoxybenzyl)oxy)-2,8,12-trimethyltetradeca-2,8,12-trien-5-ol (115 mg, 132 μmol, 1.0 eq.) for 30 min. Chloromethyl methyl sulfide (22.1 μl, 264 μmol, 2.0 eq.) was added and the mixture was stirred at 0° C. for 3 h. After quench with saturated NH₄Cl, the aqueous layer was extracted multiple times with Et₂O. The combined organic phases were washed with brine, dried (Na₂SO₄) and concentrated. The residue was purified using column chromatography to give the product (121 mg, 129 μmol, 98%).

### (19) Preparation of (2Z,6R,7R,8E,11R,12Z)-6-(benzyloxy)-11-((tert-butyldimethyl-silyl)oxy)-7-((tert-butyldiphenylsilyl)oxy)-2,8,12-trimethyl-5-((methylthio) methoxy)tetradeca-2,8,12-trien-1-ol^{[26]}

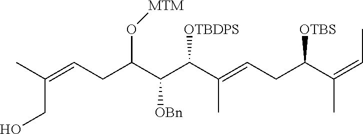

(6R,7R,11R,E)-6-(benzyloxy)-11-((Z)-but-2-en-2-yl)-7-((tert-butyldiphenylsilyl)oxy)-5-((Z)-4-((4-methoxybenzyl)oxy)-3-methylbut-2-en-1-yl)-8,13,13,14,14-pentamethyl-4,12-dioxa-2-thia-13-silapentadec-8-ene (121 mg, 129 μmol, 1.0 eq.) was dissolved in CH₂Cl₂ (5.7 ml) and H₂O (0.57 ml). DDQ (29.3 mg, 129 μmol, 1.0 eq.) was added at 0° C. and the mixture was stirred for 1 h at 0° C. The reaction was quenched by addition of saturated Na₂SO₃ solution (8.6 ml). After extraction of the aqueous phase with CH₂Cl₂ (3×30 ml) the combined organic phases were dried (MgSO₄) and concentrated. Purification by flash chromatography yielded the product (95.6 mg, 117 μmol, 91%).

### (20) Preparation of (2Z,6R,7R,8E,11R,12Z)-6-(benzyloxy)-11-((tert-butyldimethyl silyl)oxy)-7-((tert-butyldiphenylsilyl)oxy)-2,8,12-trimethyl-5-((methylthio) methoxy) tetradeca-2,8,12-trienoic Acid^{[27]}

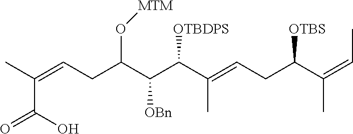

A) To a solution of (2Z,6R,7R,8E,11R,12Z)-6-(benzyloxy)-11-((tert-butyldimethyl-silyl)oxy)-7-((tert-butyldiphenylsilyl)oxy)-2,8,12-trimethyl-5-((methylthio) methoxy)tetradeca-2,8,12-trien-1-ol (95.6 mg, 117 μmol, 1.0 eq.) in CH₂Cl₂ (1.2 ml) was added MnO₂ (101 mg 1.17 mmol, 10 eq.). The reaction was stirred for 2 h at room temperature and filtered through a Celite pad. The product (92.9 mg, 114 μmol, 98%) was obtained by concentration of the filtrate and purification via column chromatography.

B) The obtained aldehyde (92.9 mg, 114 μmol, 1.0 eq.) was dissolved in tert-butyl alcohol (1.5 ml). 2-methyl-2-butene (0.85 ml, 7.98 mmol, 70 eq.), NaClO₂ (227 mg, 2.51 mmol, 22 eq.) and sodium dihydrogenphosphate (227 mg, 1.89 mmol, 17 eq.) in H₂O (1.5 ml) were added. The reaction was stirred for 16 h at room temperature. 0.5 M KHSO₄ and EtOAc were added, the phases separated and the aqueous phase was washed with EtOAc. The combined organic phases were washed with 10% NaHSO₃ and brine, dried (Na₂SO₄) and concentrated under reduced pressure. The product (91.0 mg, 110 μmol, 96%) was obtained by column chromatography.

### (21) Preparation of (2Z,6R,7R,8E,11R,12Z)-6-(benzyloxy)-7-((tert-butyldiphenyl-silyl)oxy)-11-hydroxy-2,8,12-trimethyl-5-((methylthio)methoxy)tetradeca-2,8,12-trienoic Acid^{[28]}

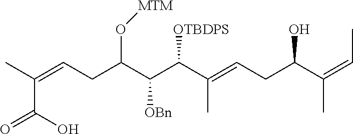

(2Z,6R,7R,8E,11R,12Z)-6-(benzyloxy)-11-((tert-butyldimethylsilyl)oxy)-7-((tert-butyldiphenylsilyl)oxy)-2,8,12-trimethyl-5-((methylthio)methoxy)tetradeca-2,8,12-trienoic acid (91.0 mg, 110 μmol, 1.0 eq.) was dissolved in ethanol (0.6 ml). The reaction was stirred at 55° C. for 2 h after addition of PPTS (8.26 mg, 32.9 μmol, 0.3 eq.). After removal of the solvent under reduced pressure, the residue was dissolved in EtOAc, washed with brine, H₂O and dried (MgSO₄). The product (64.4 mg, 89.8 μmol, 82%) was obtained by concentration of the organic phase, followed by column chromatography.

### (22) Preparation of (3Z,7R,8R,9E,12R)-7-(benzyloxy)-12-((Z)-but-2-en-2-yl)-8-((tert-butyldiphenylsilyl)oxy)-3,9-dimethyl-6-((methylthio)methoxy)oxacyclodo-deca-3,9-dien-2-one^{[29]}

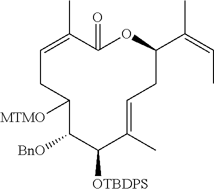

A mixture of (2Z,6R,7R,8E,11R,12Z)-6-(benzyloxy)-7-((tert-butyldiphenylsilyl)oxy)-11-hydroxy-2,8,12-trimethyl-5-((methylthio)methoxy)tetradeca-2,8,12-trienoic acid (64.4 mg, 89.8 μmol, 1.0 eq.), DIPEA (0.61 ml, 3.59 mmol, 40 eq.) and 2,4,6-trichlorbenzoylchloride (0.28 ml, 1.80 mmol, 20 eq.) in THF (4 ml) was stirred overnight at room temperature. The mixture was diluted with benzene (12 ml) and added to a solution of DMAP (548 mg, 4.49 mmol, 50 eq.) in benzene (55 ml) at 80° C. over a period of 12 h. After stirring for another hour, the reaction was quenched by addition of saturated NaHCO₃. The aqueous phase was extracted with EtOAc (2×25 ml). The combined organic phases were dried (Na₂SO₄) and concentrated. The product (34.5 mg, 49.4 μmol, 55%) was obtained by column chromatography.

### (23) Preparation of (3Z,7R,8R,9E,12R)-12-((Z)-but-2-en-2-yl)-8-((tert-butyldiphenylsilyl)oxy)-7-hydroxy-3,9-dimethyl-6-((methylthio)methoxy)oxa-cyclododeca-3,9-dien-2-one^{[30]}

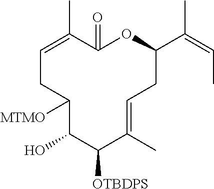

To a solution of di-tert-butylbiphenyl (263 mg, 988 μmol, 20 eq.) in THF (4.0 ml) was added activated lithium wire (24.0 mg, 3.46 mmol, 70 eq.) at 0° C. to give a green solution. The mixture was used to titrate a solution of (3Z,7R,8R,9E,12R)-7-(benzyloxy)-12-((Z)-but-2-en-2-yl)-8-((tert-butyldiphenylsilyl)oxy)-3,9-dimethyl-6-((methylthio)methoxy)oxacyclododeca-3,9-dien-2-one (34.5 mg, 49.4 μmol, 1.0 eq.) in THF (4.0 ml) at −78° C. until the green color persisted (3 h). The reaction was quenched by addition of saturated NH₄Cl (9.0 ml). Et₂O (9.0 ml) was added and the phases were separated, followed by extraction of the aqueous phase with Et₂O (3×). The combined organic phases were dried (MgSO₄) and concentrated. The product (29.7 mg, 46.9 μmol, 95%) was obtained by column chromatography of the residue.

### (24) Preparation of (3Z,7R,8R,9E,12R)-12-((Z)-but-2-en-2-yl)-8-((tert-butyldiphenylsilyl)oxy)-3,9-dimethyl-6-((methylthio)methoxy)-2-oxooxacyclo-dodeca-3,9-dien-7-yl 3-methylbutanoate^{[31]}

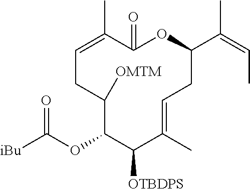

A mixture of (3Z,7R,8R,9E,12R)-12-((Z)-but-2-en-2-yl)-8-((tert-butyldiphenylsilyl)oxy)-7-hydroxy-3,9-dimethyl-6-((methylthio)methoxy)oxacyclododeca-3,9-dien-2-one (29.7 mg, 46.9 μmol, 1.0 eq.), pyridine (19.0 μl, 235 μmol, 5.0 eq.), DMAP (1.15 mg, 9.38 μmol, 0.2 eq.) and 3-methylbutanoyl chloride (28.9 μl, 235 μmol, 5.0 eq.) was stirred overnight at room temperature. The reaction mixture was diluted with CH₂Cl₂, washed with 1 M HCl, saturated NaHCO₃ and H₂O. The combined organic phases were dried (Na₂SO₄) and the solvent was removed under reduced pressure. The residue was purified using column chromatography to give the product (27.9 mg, 44.1 μmol, 94%)

### (25) Preparation of (3Z,7R,8R,9E,12R)-12-((Z)-but-2-en-2-yl)-8-((tert-butyldiphenylsilyl)oxy)-6-hydroxy-3,9-dimethyl-2-oxooxacyclododeca-3,9-dien-7-yl 3-methylbutanoate^{[32]}

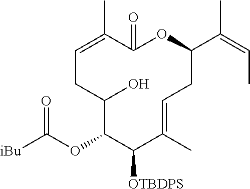

(3Z,7R,8R,9E,12R)-12-((Z)-but-2-en-2-yl)-8-((tert-butyldiphenylsilyl)oxy)-3,9-dimethyl-6-((methylthio)methoxy)-2-oxooxacyclododeca-3,9-dien-7-yl 3-methylbutanoate (27.9 mg, 44.1 μmol, 1.0 eq.) was dissolved in a THF/H₂O mixture (4:1). After addition of 2,6-lutidine (15.4 μl, 132.3 μmol, 3.0 eq.) and AgNO₃ (37.5 mg, 220.5 μmol, 5.0 eq.), the mixture was stirred at room temperature for 45 min. After addition of Et₂O the solution was filtered through Celite. The combined organic phases were washed with saturated aqueous CuSO₄ solution (2×), H₂O (1×) and then dried (K₂CO₃). The residue obtained by removal of the organic solvent was purified using column chromatography, yielding clean product (24.6 mg, 38.8 μmol, 88%).

### (26) Preparation of (3Z,7S,8R,9E,12R)-12-((Z)-but-2-en-2-yl)-8-((tert-butyldiphenyl-silyl)oxy)-3,9-dimethyl-2,6-dioxooxacyclododeca-3,9-dien-7-yl-3-methylbutanoate^{[33]}

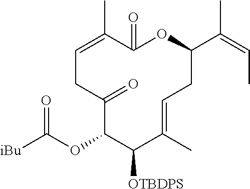

To a solution of oxalyl chloride (51 μl, 73.8 μmol, 2.0 eq.) at −78° C. in dichloromethane (4.4 μl) was added a solution of DMSO (10 μl, 147 μmol, 4.0 eq.) in dichloromethane (45 μl) over 5 min. The reaction mixture was stirred for another 10 min. A solution of (3Z,7R,8R,9E,12R)-12-((Z)-but-2-en-2-yl)-8-((tert-butyldiphenylsilyl)oxy)-6-hydroxy-3,9-di-methyl-2-oxooxacyclododeca-3,9-dien-7-yl 3-methylbutanoate (24.6 mg, 38.8 μmol, 1.0 eq.) in dichloromethane (47 μl) was added to the mixture over 5 min. After 1 hour at −78° C. triethylamine (32 μl, 233 μmol, 6.0 eq.) was added and the solution was allowed to warm up to room temperature. Addition of CH₂Cl₂ (233 μl) was followed by washing with H₂O. The organic phase was dried (MgSO₄) and concentrated. Column chromatography gave the product (23.3 mg, 36.9 μmol, 95%) in good yield.

### (27) Preparation of (3Z,7S,8R,9E,12R)-12-((Z)-but-2-en-2-yl)-8-hydroxy-3,9-dimethyl-2,6-dioxooxacyclododeca-3,9-dien-7-yl 3-methylbutanoate^{[20]}

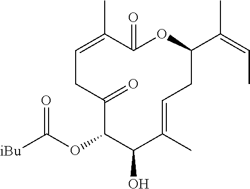

(3Z,7S,8R,9E,12R)-12-((Z)-but-2-en-2-yl)-8-((tert-butyldiphenylsilyl)oxy)-3,9-dimethyl-2,6-dioxooxacyclododeca-3,9-dien-7-yl 3-methylbutanoate (23.3 mg, 36.9 μmol, 1.0 eq.) was dissolved in THF (1.0 ml) at 0° C. A 1 M TBAF solution in THF (0.22 ml, 221 μmol, 6.0 eq.) was added dropwise. The reaction was allowed to warm to room temperature over 3 h and was stirred for another 4 h. The reaction was quenched by addition of a saturated NaHCO₃. The aqueous solution was extracted with EtOAc (4×). The combined organic phases were dried (MgSO₄) and concentrated. The product (14.2 mg, 36.2 μmol, 98%) was obtained by column chromatography of the residue.

### (28) Preparation of (3Z,7S,8R,9E,12R)-8-(((2S,3S,4R,5R)-3,4-bis((acetyl-12-chloranyl)oxy)-5-(((acetyl-12-chloranyl)oxy)methyl)tetrahydrofuran-2-yl)oxy)-12-((Z)-but-2-en-2-yl)-3,9-dimethyl-2,6-dioxooxacyclododeca-3,9-dien-7-yl 3-methylbutanoate^{[34]}

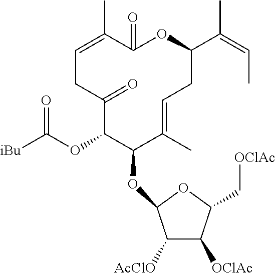

A mixture of (3Z,7S,8R,9E,12R)-12-((Z)-but-2-en-2-yl)-8-hydroxy-3,9-dimethyl-2,6-dioxooxacyclododeca-3,9-dien-7-yl 3-methylbutanoate (14.2 mg, 36.2 μmol, 1.0 eq.) was dissolved in dichloromethane (0.30 ml), together with molecular sieves 4 Å (30.0 mg), Hg(CN)₂ (17.4 mg, 68.8 μmol, 1.9 eq.) and HgBr₂ (6.52 mg, 18.1 μmol, 0.5 eq.). The mixture was stirred for 3 h at room temperature. A solution of (3S,4R,5R)-4-((acetyl-12-chloranyl)oxy)-5-(((acetyl-12-chloranyl)oxy)methyl)-2-bromotetrahydrofuran-3-yl 2-chloroacetate (19.3 mg, 43.4 μmol, 1.2 eq.) in dichloromethane (0.03 ml) was added and subsequently stirred for 4 d at room temperature. The mixture was filtered, washed with aqueous KI solution and H₂O, dried (NaSO₄) and the solvent was removed under reduced pressure. The residue was purified using column chromatography to give the product (17.8 mg, 23.5 μmol, 65%).

### (29) Preparation of Disciformycin^{[35]}

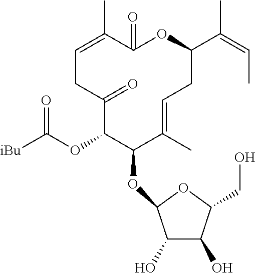

To a solution of (3Z,7S,8R,9E,12R)-8-(((2S,3S,4R,5R)-3,4-bis((acetyl-12-chloranyl)oxy)-5-(((acetyl-12-chloranyl)oxy)methyl)tetrahydrofuran-2-yl)oxy)-12-((Z)-but-2-en-2-yl)-3,9-di-methyl-2,6-dioxooxacyclododeca-3,9-dien-7-yl 3-methylbutanoate (17.8 mg, 23.5 μmol, 1.0 eq.) in THF/H₂O (1.3 ml, 2:1) was added LiOH.H₂O (8.89 mg, 211 μmol, 9.0 eq.) at 0° C. After stirring at room temperature for 3 h, THF was removed under reduced pressure at ambient temperature. The residue was extracted with EtOAc multiple times, the combined organic phases were dried (MgSO₄) and concentrated. The product (10 mg, 19.1 μmol, 81%) was obtained after column chromatography.

### Example 6 Biosynthesis and Biophysical Analysis of Gulmirecin A and B

Production and Isolation:

For the production of gulmirecins, P. fallax strain HKI 727 (DSM 28991) was cultured at 30° C. in Erlenmeyer flasks under oxic conditions with gentle shaking (130 rpm). The broth of a seven day old culture (50 l total volume) grown in MD1 medium (casitone 0.3% (w/v), CaCl₂×2 H₂O 0.07% (w/v), MgSO₄×7 H₂O 0.2% (w/v), vitamin B₁₂ 0.00005% (w/v), and 1 ml trace elements solution SL-4 consisting of EDTA 0.05% (w/v), FeSO₄×7 H₂O 0.02% (w/v), ZnSO₄×7 H₂O 0.001% (w/v), MnCl₂×4 H₂O 0.0003% (w/v), H₃BO₃ 0.003% (w/v), CoCl₂×6 H₂O 0.020% (w/v), CuCl×2H₂O 0.0001% (w/v), NiCl₂×6 H₂O 0.0002% (w/v), and Na₂MoO₄×2 H₂O 0.0003% (w/v)) was filtered through a cellulose-based filter paper (retention capacity 12-25 μm) in order to remove the cell biomass. The filtrate was extracted three times with equivalent volumes of ethyl acetate. The organic layers were combined and residual water was removed following the addition of anhydrous sodium sulfate (30 g/l) by another filtration step. Subsequently, the organic extract was concentrated under reduced pressure. The residue was dissolved in a small amount of methanol and subjected to flash column chromatography using 45 g of Polygoprep 60-50 C₁₈ (Macherey-Nagel) as a stationary phase. To this end, the octadecyl phase had been suspended in 20% aqueous methanol and filled into a glass column (30×3 cm). Elution started with 150 ml of 20% methanol and was continued with the same volume of 40%, 60%, 80%, and 100% methanol. Antimicrobial activity screening against S. aureus indicated the 80% methanol fraction to contain the bioactive compounds. Furthermore, ¹H NMR analysis revealed the same fraction to feature unique signals in the olefinic range between 5.00 and 6.00 ppm.

Isolation of the gulmirecins was accomplished by two consecutive reverse-phase HPLC steps. The initial separation was conducted on a Nucleodur PFP column (250×10 mm, 5 μm; Macherey-Nagel) using a linear gradient of methanol in water+0.1% trifluoroacetic acid and a flow rate of 2 ml/min: 10% methanol for 3 min, 10%→100% methanol within 30 min, 100% methanol for 10 min. The gulmirecin-containing fraction was collected between 28 and 32 min post injection. Final purification was achieved on a Nucleodur C₁₈ HTec column (250×10 mm, 5 μm; Macherey-Nagel) using an isocratic flow (2 ml/min) of 70% methanol in water+0.1% trifluoroacetic acid. Under these conditions, 3 had a retention time of 11.5 min and 4 possessed a retention time of 14.0 min. The elution of gulmirecins was detected by wavelength monitoring at 210 nm using a diode array detector. A total of 8.2 mg of gulmirecin A (3) and 2.1 mg of gulmirecin B (4) were isolated in this way.

Gulmirecin A (3):

[α]²⁵_{D}+98.4 (c 1.0, MeOH); UV (MeOH) λₘₐₓ (log ε) 203 (4.28); IR (film) νₘₐₓ 3344, 2938, 1733, 1456, 1374, 1251, 1167, 1074, 1022, 863, 792 cm⁻¹; HRESIMS m/z 541.2660 [M−H]⁻, calcd 541.2654 for C₂₇H₄₁O₁₁.

TABLE 5
NMR spectroscopic data of gulmirecin A (3) in
chloroform-d₁ (¹H at 500 MHz, ¹³C at 125 MHz).

| | | δ_{H}, mult. | | |
|---|---|---|---|---|
| Pos. | δc | (J in Hz) | HMBC | NOESY |
| 1 | 174.3 | | | |
| 2 | 45.1 | 2.76, dq (10.4, 6.7) | 1, 3, 15 | 15 |
| 3 | 68.9 | 3.84, ddd (10.4, 4.0, 3.2) | 2, 5 | 4a, 4b, 15 |
| 4 | 44.3 | a: 3.03, dd (20.2, 3.2) | 2, 3, 5 | 3, 4b |
| | | b: 2.79, dd (20.2, 4.0) | 5 | 3, 4a, 7, 15 |
| 5 | 203.5 | | | |
| 6 | 80.4 | 5.01, d (9.2) | 5, 7,23 | 16 |
| 7 | 83.5 | 4.18, d (9.2) | 6, 8, 9, 16, 18 | 4b, 9, 18 |
| 8 | 133.1 | | | |
| 9 | 129.9 | 5.46, ddd (11.5, 3.3, 1.5) | 7, 16 | 7, 10b, 11 |
| 10 | 31.9 | a: 2.67, dt (14.5, 11.5) | 8, 9, 11, 12 | 10b, 16 |
| | | b: 1.98, m | 8, 9, 12 | 9, 10a, 11 |
| 11 | 70.9 | 5.82, dd (11.5, 1.6) | 1, 10, 13, 17 | 9, 10b, 17 |
| 12 | 132.9 | | | |
| 13 | 123.6 | 5.35, dq (6.9, 1.6) | 14, 17 | 17 |
| 14 | 13.0 | 1.65, dd (6.9, 1.6) | 12, 13 | |
| 15 | 15.2 | 1.23, d (6.7) | 1, 2, 3 | 2, 3, 4b |
| 16 | 11.4 | 1.70, t (1.5) | 7, 8, 9 | 6, 10a |
| 17 | 18.3 | 1.67, t (1.6) | 11, 12, 13 | 11, 13 |
| 18 | 108.2 | 5.12, s | 7, 20, 21 | 7, 19 |
| 19 | 78.7 | 3.97, d (1.0) | | 18 |
| 20 | 78.0 | 3.98, dd (2.0, 1.0) | 22 | |
| 21 | 87.7 | 4.09, q (2.0) | 20, 22 | 22 |
| 22 | 61.8 | 3.78, dt (11.7, 2.0) | 20, 21 | 21 |
| 23 | 172.6 | | | |
| 24 | 42.8 | 2.26, d (7.1) | 23, 25, 26, 27 | 26, 27 |
| 25 | 25.7 | 2.09, m | 23, 24, 26, 27 | 26, 27 |
| 26 | 22.3 | 0.96, d (6.6) | 24, 25, 27 | 24, 25 |
| 27 | 22.3 | 0.96, d (6.6) | 24, 25, 26 | 24, 25 |

Gulmirecin B (4):

[α]^{25D}+113.5 (c 0.9, MeOH); UV (MeOH) λₘₐₓ (log ε) 202 (4.19); IR (film) νₘₐₓ 3306, 2929, 1717, 1652, 1558, 1539, 1506, 1456, 1376, 1175, 1020, 876 cm⁻¹; ¹H NMR (500 Mhz, methanol-d₄) δ_{H} [ppm] (J [Hz]) 1.08 (3H, d, J 6.9, H-15), 1.63 (3H, d, J 1.4, H-16), 1.66 (2H, m, H-3), 1.68 (3H, m, H-17), 1.69 (3H, dd, J 7.0, 1.6, H-14), 1.94 (1H, m, H-10b), 2.46 (1H, ddd, J 20.5, 10.8, 4.2, H-4b), 2.66 (1H, dt, J 14.4, 11.8, H-10a), 2.73 (1H, m, H-2), 2.91 (1H, dt, J 20.5, 4.2, H-4a), 3.59 (1H, dd, J 11.8, 5.4, H-22b), 3.67 (1H, dd, J 11.8, 3.4, H-22a), 3.81 (1H, dd, J 5.8, 3.3, H-20), 3.90 (1H, ddd, J 5.8, 5.4, 3.4, H-21), 4.02 (1H, d, J 8.5, H-7),4.06 (1H, dd, J 3.3, 1.3, H-19), 4.07 (1H, d, J 8.5, H-6), 5.04 (1H, d, J 1.3, H-18), 5.37 (1H, dq, J 7.0, 1.6, H-13), 5.38 (1H, m, H-9), 5.87 (1H, dd, J 11.8, 2.8, H-11); ¹³C NMR (125 MHz, methanol-d₄) δ_{C} [ppm] 11.7 (C-16), 13.1 (C-14), 18.4 (C-17), 18.8 (C-15), 28.0 (C-3), 32.6 (C-10), 35.6 (C-4), 39.0 (C-2), 63.0 (C-22), 72.3 (C-11), 78.8 (C-20), 81.7 (C-6), 83.2 (C-19), 86.3 (C-21), 88.7 (C-7), 109.9 (C-18), 123.9 (C-13), 129.3 (C-9), 135.0 (C-12), 136.4 (C-8), 177.8 (C-1), 210.1 (C-5); HRESIMS m/z 465.2098 [M+Na]⁺, calcd 465.2095 for C₂₂H₃₄O₉Na.

### Example 7 Assessment of Antimicrobial Activity of Gulmirecin A and B

Agar Diffusion Assay.

Antimicrobial activities of the gulmirecins were determined in a primary screen against Bacillus subtilis ATCC 6633, Staphylococcus aureus SG511, Staphylococcus auricularis DSM 20609, Mycobacterium vaccae IMET 10670, Pseudomonas aeruginosa K799/61, Escherichia coli SG458, Sporobolomyces salmonicolor SBUG 549, Candida albicans ATCC14053 and Penicillium notatum JP 36. To this end, holes with 7 mm diameter were aseptically punched in the respective agar medium. Subsequently, the agar plates were inoculated with the test organisms. 0.5 mg of every test compound was dissolved in 1 mL of methanol, and 50 μL of this solution was transferred to a single hole. Ciprofloxacin and amphotericin B served as positive controls. After evaporation of the solvent, the agar plates were incubated depending on the growth conditions of the test organisms. A noticeable antimicrobial activity resulted in an inhibition zone of >10 mm. The test results are shown in Table 6 below, wherein the given values represent the diameters of the respective inhibition zone in the agar diffusion assay.

TABLE 6
Antimicrobial activity of gulmirecin A (3) and B (4)

| Microorganism | 3 | 4 | Ciprofloxacin | Amphotericin B |
|---|---|---|---|---|
| Bacillus subtilis | 35 | n.a. | 30 | n.a. |
| Staphylococcus aureus | 29 | 17 | 19 | n.a. |
| Staphylococcus auricularis | 31 | 19 | 20 | n.a. |
| Mycobacterium vaccae | 17 | n.a. | 24 | n.a. |
| Pseudomonas aeruginosa | n.a. | n.a. | 38 | n.a. |
| Escherichia coli | n.a. | n.a. | 34 | n.a. |
| Sporobolomyces salmonicolor | n.a. | n.a. | n.a. | 19 |
| Candida albicans | n.a. | n.a. | n.a. | 22 |
| Penicillium notatum | n.a. | n.a. | n.a. | 18 |

n.a., no activity observed

As demonstrated above, gulmirecin A (3) and B (4) have an excellent antimicrobial activity against Gram-positive bacteria, and particularly against staphylococci. However, they were inactive against Gram-negative bacteria, including Escherichia coli and Pseudomonas aeruginosa. Fungi, such as Candida albicans, Penicillium notatum, and Sporobolomyces salmonicolor, were also not affected by the gulmirecins.

Bouillon Dilution Assay.

To determine the MIC₉₀ value of 3, the test organism was cultured in 500 μl aliquots of trypric soy broth (peptone from casein 1.7% (w/v), peptone from soymeal 0.3% (w/v), D-(+)-glucose 0.25% (w/v), NaCl 0.5% (w/v), K₂HPO₄ 0.25% (w/v), pH 7.3) at 37° C. for 16 h. Before incubation, 3 was added in decreasing concentrations (100 g/ml to 12.5 μg/ml). After incubation, the OD₆₀₀ was measured. The experiment was run in triplicate. Ciprofloxacin was used as a positive control.

The MIC₉₀ values of 3 against methicillin-resistant S. aureus (MRSA) and S. auricularis A were 23.05 μM and 22.48 μM, respectively. Ciprofloxacin, which served as a reference, exhibited an MIC₉₀ of 75.4 μM against the MRSA strain in the same test series.

### Example 8 Assessment of Cytotoxic Effects of Gulmirecin A and B

The cytotoxic activity of gulmirecin A (3) and gulmirecin B (4) against human cells was evaluated in the well-established MTT assay using primary monocytes from human peripheral blood as well as transformed lung epithelial cell A-549 cell and leukemic Mono Mac 6 cells. Furthermore, 3 and 4 were tested against K-562, HUVEC, and HeLa cells.

Isolation of Primary Monocytes, Cell Lines and Growth Conditions.

Human primary monocytes were isolated from leukocyte concentrates, obtained from the Institute of Transfusion Medicine at the University Hospital Jena, Germany. The concentrates were prepared from the blood of healthy adult human donors who had not taken any anti-inflammatory medication for the 10 days prior to blood donation, as described. In brief, freshly withdrawn peripheral blood was pretreated with citrate-phosphate-dextrose solution as anticoagulant and processed with the 2C+ protocol of the Atreus Whole Blood Processing System (Terum BCT). Peripheral blood mononuclear cells (PBMC) were isolated by dextran sedimentation and centrifugation on LSM 1077 lymphocyte separation medium (PAA Laboratories). For isolation of monocytes, the PBMC were washed twice with ice-cold phosphate buffered saline (PBS) and plated (density 2×10⁷ cells/ml) in culture flasks (Greiner Bio-One) containing PBMC medium (RPMI 1640 medium supplemented with 100 U/ml penicillin, 100 μg/ml streptomycin and 2 mM L-glutamine) for 1.5 hours at 37° C., 5% CO₂. Non-adherent cells were removed; adherent monocytes were scraped, washed with ice-cold PBS and resuspended in ice-cold PBS with a purity of >85%, defined by forward- and side-light scatter properties and detection of the CD14 surface molecule by flow cytometry (BD FACS Calibur). Monocytes were cultured in monocyte medium (RPMI 1640 supplemented with 2% heat-inactivated fetal calf serum (FCS, 10%, v/v), L-glutamine (2 mM), penicillin (100 U/ml), and streptomycin (100 μg/ml)).

The human monocytic cell line Mono Mac 6 was cultured in RPMI 1640 medium supplemented with FCS (10%, v/v), penicillin (100 U/ml), streptomycin (100 μg/ml), insulin (10 μg/ml), oxaloacetic acid (1 mM), sodium pyruvate (1 mM), and 1× non-essential amino acids at 37° C. and 5% CO₂. A-549 cells (DSM ACC-107) were cultured in DMEM high glucose (4.5 g/l) medium supplemented with FCS (10% v/v), penicillin (100 U/ml) and streptomycin (100 μg/ml) at 37° C. in a 5% CO₂ incubator. K-562 (DSM ACC 10) and HeLa cells (DSM ACC 57) were grown in RPMI 1640, whereas cells of HUVEC (ATCC CRL-1730) were cultured in DMEM medium. The respective media were supplemented with ultraglutamine 1 (10 ml/l), gentamicin sulfate (500 μl/l), and FCS (10% v/v).

MTT Assay.

Cells (3×10⁵ Mono Mac 6, 1×10⁵ A-549 cells, or 2×10⁶ monocytes per well) were seeded in a 96-well plate in the respective medium (100 μl/well). Monocytes were allowed to adhere for 1.5 hours (37° C., 5% CO₂) prior to treatment. Test compounds (0.3% DMSO as vehicle) were added to each well and samples were incubated for 48 hrs. Then, 20 μl of thiazolyl blue tetrazolium bromide (MTT, 5 mg/ml PBS) were added, and the incubation was continued at 37° C., 5% CO₂ until blue staining of the vehicle control. Formazan formation was stopped by adding 100 μl of lysis buffer (SDS, 10%, w/v in 20 mM HCl) and samples were shaken overnight. Absorbance of each well was measured at 570 nm in a Multiskan™ microplate spectrophotometer (Thermo Scientific).

Incubation of the cells with either 3 or 4 for 24 or 48 hours caused no reduction in cell viability at the concentrations required for antibacterial activity. In contrast, the reference compound staurosporine was highly cytotoxic for all three cell types.

Evaluation of Antiproliferative and Cytotoxic Effects.

The test substances were dissolved in methanol before being diluted in DMEM. The adherent cells were harvested at the logarithmic growth phase after soft trypsinization using 0.25% trypsin in PBS containing 0.02% EDTA. For each experiment, approximately 10,000 cells were seeded with 0.1 ml culture medium per well of the 96-well microplates. HeLa cells were pre-incubated for 48 h prior to the addition of the test compounds, which were carefully diluted on the subconfluent monolayers. Incubation was then conducted in a humidified atmosphere at 37° C. and 5% CO₂. In case of K-562 cells, the number of viable cells in every well was determined using the CellTiter-Blue1 assay. The adherent HUVEC and HeLa cells were fixed by glutaraldehyde and stained with a 0.05% solution of methylene blue for 15 min. After gently washing, the stain was eluted with 0.2 ml of 0.33 N HCl in the wells. The optical densities were measured at 660 nm in a SUNRISE microplate reader (TECAN).

3 and 4 did not inhibit the proliferation of K-562, HUVEC, or HeLa cells at a concentration of 100 μM.

### Example 9 Identification and Analysis of the Gul Gene Cluster

Retrobiosynthetic analysis of 3 suggests that its polyketide portion is assembled from an acetate starter unit and six polyketide extender molecules, including three malonyl-CoAs and three methylmalonyl-CoAs. Assuming a co-linear biosynthesis, the genome of P. fallax HKI 727 (DSM 28991) was screened for gene loci featuring seven PKS modules. The gulmirecin (gul) gene cluster was identified taking the substrate specificities of the gate-keeping acyl transferase (AT) domains in every PKS module and the reductive domains into consideration. The gul gene cluster includes six PKS genes (gulA-gulF) having the organization shown in FIG. 4.

The oxygen-bearing stereogenic centers of 3 at C-3, C-7 and C-11 are introduced at different extension steps by NADPH-dependent reduction of the respective Claisen products. They are catalyzed by the ketoreductase (KR) domains of the PKS modules, which can be classified into two groups (A-type and B-type) on the basis of their distinct substrate orientation. Since the relative position of the substrate determines the stereochemical outcome of the reduction, the stereospecificity of KR domains can be inferred from their 3D architecture. By using a sequence-based model, a 3R, 7S, 11R configuration for the macrolide that is offloaded from the PKS assembly line was predicted. While the predictions for the chiral centers at C-3 and C-11 already matched the NOE-derived stereochemistry, the discrepancy at C-7 can be rationalized. The 7S configuration was predicted under the assumption that the AT domain of GulD selects malonyl-CoA as an extender unit. This means that the hydroxyl group at C-6 is not introduced during the GulD-catalyzed polyketide chain extension, but results from an independent reaction at a later biosynthetic stage. The cytochrome P450 GulG is a likely candidate for the expected hydroxylation due its sequence homology to macrolide carbon hydroxylases, such as EryK. Once the hydroxyl group is installed, the original PKS-derived 7S configuration will switch to 7R.

The structure of 3 suggests the skipping of two reductive domains in the PKS GulF during its assembly.

### Example 10 Assessment of Resistance Mutations in S. aureus

The reference strain S. aureus N315 (genome accession number NC_002745) has been sequentially exposed to increasing concentrations of disciformycin A (1). Resistant mutants developed at a frequency of ca. 10^{-s} as determined by colony-forming units (cfu) count of a defined inoculum treated with the 8×MIC of disciformycin A (1). Finally, ten independent resistant mutants were obtained that grew in the presence of 50 μg/mL disciformycin A (1). These mutants were analyzed by whole-genome sequencing and comparison to the wildtype reference genome. The results are shown in Table 7.

TABLE 7
Mutations (amino acid changes) found in Disciformycin-
resistant S. aureus N315 mutants.

| Mutant # | RpoB (β subunit) | RpoC (β′subunit) |
|---|---|---|
| Mt50DscA.1 | Q575R, C593W, R594C | — |
| Mt50DscA.2 | Y507D, D611N | — |
| Mt50DscA.3 | — | H785R, T936A |
| Mt50DscA.4 | D611N | — |
| Mt50DscA.5 | C593W, R594C | D952N |
| Mt50DscA.6 | D611N | — |
| Mt50DscA.8 | Q575R, C593W, R594C | — |
| Mt50DscA.9 | R594C | I763M |
| Mt50DscA.10 | Q575R, C593W, R594C | — |
| Mt50DscA.11 | Y510H, C593W, R594C | — |

All of the mutations mapped to the 1 and 3′ subunit of S. aureus RNA polymerase. Importantly, no cross-resistance with rifampicin was observed. All of the Diciformycin A-resistant mutants were at least 8-fold resistant towards treatment with Disciformycins B-D and Gulmirecins A and B, which most probably exhibit the same mechanism of action. (Table 8).

TABLE 8
Susceptibility of Disciformycin A-resistant S. aureus N315 mutants
towards disciformycin and gulmirecin derivatives and rifampicin.

| S. aureus wildtype | MIC [μg/ml] | | | | | | |
|---|---|---|---|---|---|---|---|
| (WT) and mutants | DscA (1) | DscB (2) | DscC (5) | DscD (6) | GlmA (3) | GlmB (4) | RIF |
| WT | 4 | 1 | 4 | 0.5 | 4 | 32 | 0.003 |
| Mt50DscA.1 | >64 | >64 | >64 | 32 | >64 | >64 | 0.003 |
| Mt50DscA.2 | >64 | >64 | n.d. | n.d. | >64 | >64 | 0.005 |
| Mt50DscA.3 | >64 | >64 | >64 | 64 | >64 | >64 | 0.003 |
| Mt50DscA.4 | >64 | >64 | n.d. | n.d. | >64 | >64 | 0.001 |
| Mt50DscA.5 | 64 | 16 | 32 | 4 | 64 | >64 | 0.001 |
| Mt50DscA.6 | >64 | >64 | >64 | 32 | >64 | >64 | 0.001 |
| Mt50DscA.8 | >64 | >64 | n.d. | n.d. | >64 | >64 | 0.001 |
| N315 Mt50DscA.9 | >64 | >64 | n.d. | n.d. | >64 | >64 | 0.001 |
| Mt50DscA.10 | >64 | >64 | n.d. | n.d. | >64 | >64 | 0.001 |
| Mt50DscA.11 | >64 | 32 | n.d. | n.d. | >64 | >64 | 0.001 |

(Dsc: Disciformycin; Glm: Gulmirecin; RIF: rifampicin; n.d. not determined)

### Example 11 Assessment of Intra-Macrophage Activity

The PMA-differentiated human THP-1 cell line (macrophages) was used as cellular host for infection with S. aureus Newman at an MOI (multiplicity of infection) of 10. After 2 h, extracellular S. aureus cells were eliminated by lysostaphin treatment and compounds were added for 18 h at their respective 1×, 4× and 8×MIC. Intracellular bacteria were recovered and serial dilutions were streaked out on CASO-Agar for cfu counting. The reference drug rifampicin reduced the number of intracellular bacteria by ca. 4 logs (at 8×MIC). Likewise, disciformycin A and B reduced the number of intacellular bacteria by 3 logs without causing any apparent cytotoxicity (FIG. 11).

### REFERENCES

[1] a) P. K. Agrawal, Phytochemistry 1992, 31, 3307-3330; b) W. A. Bubb, Concepts Magn. Reson. A 2003, 19, 1-19.
[2] G. J. Gerwig, J. P. Kamerling, J. F. G. Vliegenthart Carbohydr. Res. 1978, 62, 349-357
[3] G. Yadav, R. S. Gokhale, D. Mohanty, Journal of molecular biology 2003, 328, 335-363.
[4] A. T. Keatinge-Clay, Chemistry & biology 2007, 14, 898-908.
[5] E. S. Duthie, J. Gen. Microbiol. 1952 7, 320-326.
[6] K. Okonogi, Y. Noji, M. Kondo, A. Imada, T. Yokota, J. Antimicrob. Chemother. 1989, 24, 637-645.
[7] a) D. J. Del Valle, M. J. Krische, J. Am. Chem. Soc. 2013, 135, 10986-10989; b) J. G. Duboudin, B. Jousseaume; A. Bonakdar, J. Organomet. Chem. 1979, 168, 227-232.
[8] J. Wennerberg, C. Olofsson, T. Frejd, J. Org. Chem. 1998, 63, 3595-3598.
[9] Y. Nakamura, M. Hirata, E. Kuwano, E. Taniguchi, Biosci. Biotechnol. Biochem. 1998, 62, 1550-1554.
[10] B. Fraser-Reid, P. Ganney, C. Ramamurty, A. M. Gómez, J. C. López, Chemical Communications 2013, 49, 3251-3253.
[11] P. Wipf, P. C. Fritch, J. Org. Chem. 1994, 59, 4875-4886.
[12] U. Vogeli, W. von Philipsborn, Org. Magn. Resonance 1975, 7, 617-627.
[13] T. Frenzel, M. Briinjes, M. Quitschalle, A. Kirschning, Org. Lett. 2006, 6, 135-138.
[14] L. A. Paquette, I. Efremov, J. Am. Chem. Soc. 2001, 123, 4492-4501.
[15] M. Bock, R. Dehn, A. Kirschning, Angew. Chem. Int. Ed. 2008, 47, 9134-9137.
[16] K. Kinoshita, P. G. Williard, C. Khosla, D. E. Cane, J. Am. Chem. Soc. 2001, 123, 2495-2502.
[17] Z. Cai, N. Yongpruksa, M. Harmata, Org. Lett. 2012, 14, 1661-1663.
[18] M. J. Martín, L. Coello, R. Fernández, F. Reyes, A. Rodríguez, C. Murcia, M. Garrazo, C. Mateo, F. Sánchez-Sancho, S. Bueno, C. de Eguilior, A. Francesch, S. Munt, C. Cuevas, J. Am. Chem. Soc. 2013, 135, 10164-10171.
[19] I. Paterson, E. A: Anderson, S. M. Dalby, J. H. Lim, P. Maltas, O. Loiseleur, J. Genovino, C. Moessner, Org. Biomol. Chem. 2012, 10, 5861-5872.
[20] J. D. Frein, R: E. Taylor, D. L. Sackett, Org. Lett. 2009, 11, 3186-3189.
[21] S. Nakamura, J. Inagaki, M. Kudo, T. Sugimoto, K. Obara, M. Nakajima, S. Hashimoto, Tetrahedron 2002, 58, 10353-10374.
[22] A. H. McNeill, E. J. Thomas, Tetrahedron 2011, 67, 257-266.
[23] S. J. Danishefsky, J. J. Masters, W. B. Young, J. T. Link, L. B. Snyder, T. V. Magee, D. K. Jung, R. C. A. Isaacs, W. G. Bornmann, C. A. Alaimo, C. A. Coburn, M. J. Di Grandi, J. Am. Chem. Soc. 1996, 118, 2843-2859.
[24] B. M. Trost, J. P. N. Papillon, T. Nussbaumer, J. Am. Chem. Soc. 2005, 127, 17921-17937.
[25] Y. Wang, M. Kurosu, Tetrahedron 2012, 68, 4797-4804.
[26] R. L. Beingessner, J. A. Farand, L. Barriault, J. Org. Chem. 2010, 75, 6337-6346.
[27] S. Shirokawa, M. Shinoyama, I. Ooi, S. Hosokawa, A. Nakazaki, S. Kobayashi, Org. Lett. 2007, 9, 849-852.
[28] C. Prakash, S. Saleh, I. A. Blair, Tetrahedron Letters 1989, 30, 19-22.
[29] B. Chatterjee, D. Mondal, S. Bera, Tetrahedron Asymetry 2012, 23, 1170-1185.
[30] S. J. Shimshock, R. E. Waltermire, P. DeShong, J. Am. Chem. Soc. 1991, 113, 8791-8796.
[31] J. Esteve, C. Jiménez, J. Nebot, J. Velasco, P. Romea, F. Urpí, Tetrahedron 2011, 67, 6045-6056.
[32] E. J. Corey, M. G. Bock, Tetrahedron Letters 1975, 38, 3269-3270.
[33] N. Yamazaki, C. Kibayashi, J. Am. Chem. Soc. 1989, 111, 1396-1408.
[34] M. Kiso, M. Fujita, M. Tanahashi, Y. Fujishima, Y. Ogawa, A. Hasegawa, Carbohydrate Research 1988, 177, 51-67.
[35] M. Yang, W. Ye, S. W. Schneller, Bioorg. Med. Chem. 2013, 21, 4374-4377.
[36] A. Untergasser, I. Cutcutache, T. Koressaar, J. Ye, B. C. Faircloth, M. Remm, S. G. Rozen, Nucleic acids research 2012, 40, e115.
[37] C. Osswald, G. Zipf, G. Schmidt, J. Maier, H. S. Bernauer, R. Muller, S. C. Wenzel, ACS synthetic biology 2014, 3, 759-772.
[38] A. A. Iniesta, F. Garcia-Heras, J. Abellon-Ruiz, A. Gallego-Garcia, M. Elias-Arnanz, Journal of bacteriology 2012, 194, 5875-5885.
[39] Y. Zhang, J. P. Muyrers, G. Testa, A. F. Stewart, Nature biotechnology 2000, 18, 1314-1317.

The features of the present invention disclosed in the specification, the claims and/or the drawings may both separately and in any combination thereof be material for realizing the invention.

1. A compound of the general formula (I): 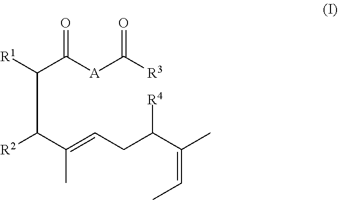
or a pharmacologically acceptable salt thereof, wherein
A represents a group of the formula: 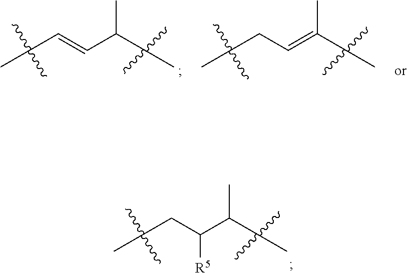
R¹, R², R³ and, if present, R⁵ each independently represents a hydrogen atom; a halogen atom; a hydroxyl group; an amino group; a mercapto group; a C₁-C₁₂ alkyl group; a C₂-C₁₂ alkenyl group; a C₂-C₁₂ alkynyl group; a heteroalkyl group containing 1 to 11 carbon atoms; a C₃-C₁₀ cycloalkyl group, a heterocycloalkyl group containing 3 to 10 ring atoms, a (C₁-C₆)alkyl-(C₃-C₇)cycloalkyl group, a(C₁-C₆)heteroalkyl-(C₃-C₇)cycloalkyl group, aC₆-C₁₄ aryl group, a heteroaryl group containing 5 to 14 ring atoms, an ar-(C₁-C₆)alkyl group, or a heteroar-(C₁-C₆)alkyl group containing 5 to 10 ring atoms;
R⁴ represents a hydrogen atom; a halogen atom; a hydroxyl group; an amino group; a C₁-C₁₂ alkyl group; or a heteroalkyl group containing 1 to 11 carbon atoms; or
R⁴ and R³ are taken together to form an oxygen or sulphur atom, or a group —NH—.

2. The compound according to claim 1, or a pharmacologically acceptable salt thereof, wherein
R¹, R² and, if present, R⁵ each independently represents a hydrogen atom; a halogen atom; a hydroxyl group; an amino group; a mercapto group; a C₁-C₁₂ alkyl group; a C₂-C₁₂ alkenyl group; a C₂-C₁₂ alkynyl group; a heteroalkyl group containing 1 to 11 carbon atoms; a C₃-C₁₀ cycloalkyl group, a heterocycloalkyl group containing 3 to 10 ring atoms, a (C₁-C₆)alkyl-(C₃-C₇)cycloalkyl group, a (C₁-C₆)heteroalkyl-(C₃-C₇)cycloalkyl group, a C₆-C₁₄ aryl group, a heteroaryl group containing 5 to 14 ring atoms, an ar-(C₁-C₆)alkyl group, or a heteroar-(C₁-C₆)alkyl group containing 5 to 10 ring atoms; and
R³ and R⁴ are taken together to form an oxygen atom.

3. The compound according to claim 1, or a pharmacologically acceptable salt thereof, wherein
R¹ and R² each independently represents a hydrogen atom; a halogen atom; a hydroxyl group; a heteroalkyl group containing 1 to 11 carbon atoms; or a heterocycloalkyl group containing 3 to 10 ring atoms.

4. The compound according to claim 1, or a pharmacologically acceptable salt thereof, wherein
R¹ represents a hydrogen atom; a hydroxyl group or a heteroalkyl group containing 1 to 11 carbon atoms; and
R² represents a hydroxyl group; or a heterocycloalkyl group containing 3 to 10 ring atoms.

5. The compound according to claim 1, or a pharmacologically acceptable salt thereof, wherein
R¹ represents a heteroalkyl group containing 1 to 11 carbon atoms; and
R² represents a heterocycloalkyl group containing 3 to 10 ring atoms.

6. The compound according to claim 1, wherein R⁵ represents a hydrogen atom; a halogen atom; a hydroxyl group; an amino group; a C₁-C₁₂ alkyl group; a C₂-C₁₂ alkenyl group; a C₂-C₁₂ alkynyl group; or a heteroalkyl group containing 1 to 11 carbon atoms.

7. The compound according to claim 1, wherein the compound is selected from the group consisting of:

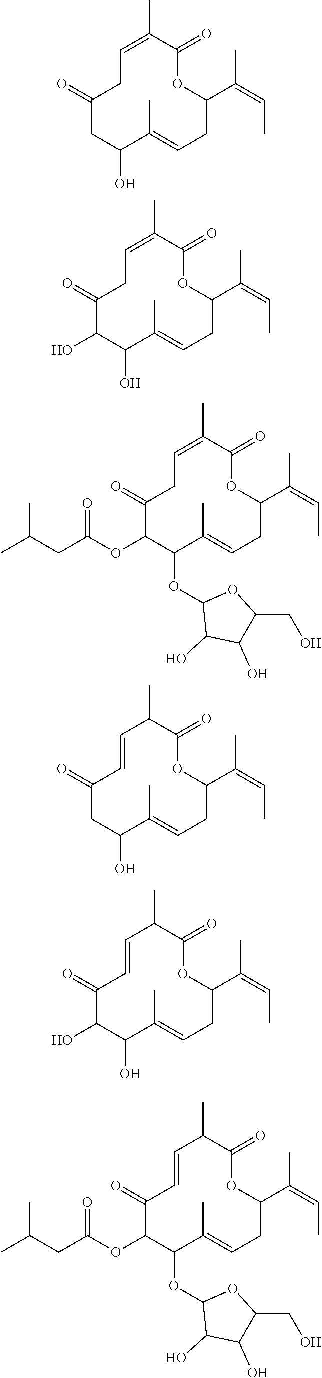

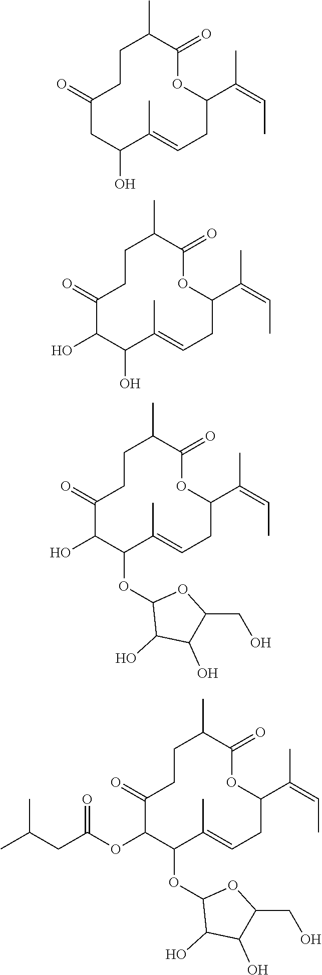

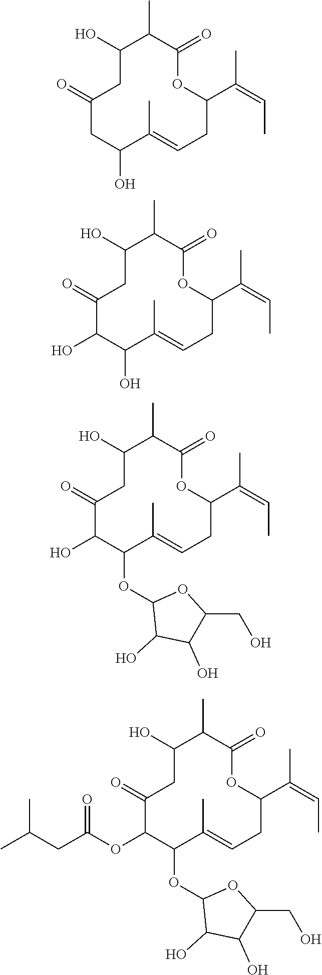

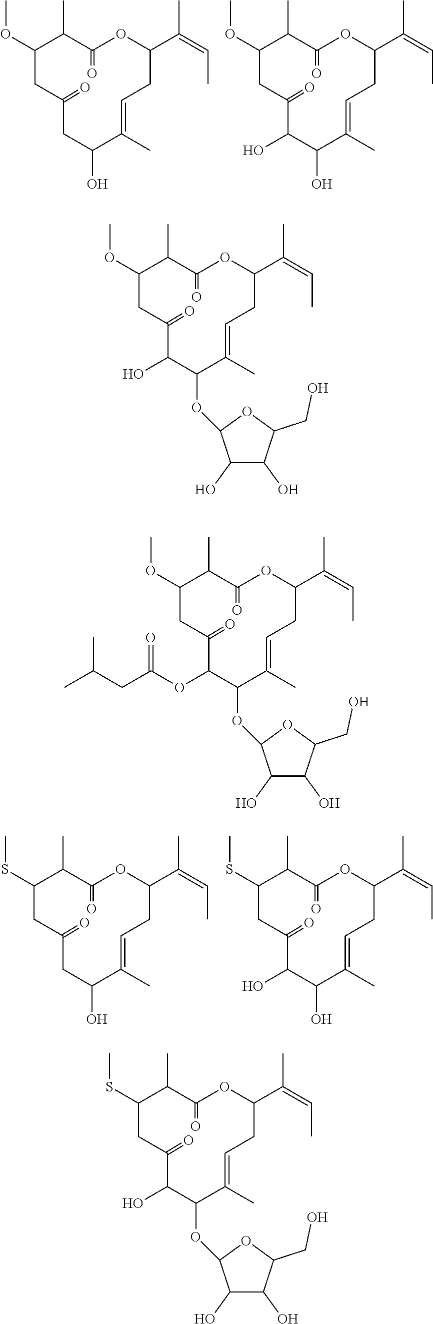

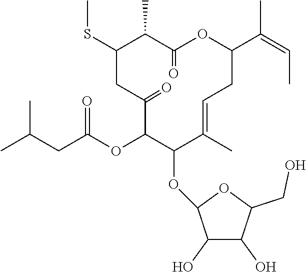 [SMILES: C/C=C(/C)C1C/C=C(\C)C(OC2OC(CO)C(O)C2O)C(OC(=O)CC(C)C)C(=O)CC(SC)[C@H](C)C(=O)O1]

8. A pharmaceutical composition comprising at least one compound according to claim 1 and, optionally, one or more carrier substance(s), excipient(s) and/or adjuvant(s).

9. A combination preparation containing at least one compound according to claim 1 and at least one further active pharmaceutical ingredient.

10-11. (canceled)

12. A recombinant polyketide synthase (PKS) capable of synthesizing a compound according to general formula (I), wherein the PKS comprises at least one polypeptide, or a functional variant thereof, according to any one of SEQ ID NOs. 11 to 19 or SEQ ID NOs. 34 to 46.

13. An isolated, synthetic or recombinant nucleic acid comprising:
(i) a sequence encoding a PKS of the invention, wherein the sequence has a sequence identity to the full-length sequence of SEQ ID NO. 1 or SEQ ID NO. 20 from at least 85%, 90%, 95%, 96%, 97%, 98%, 98.5%, 99%, or 99.5% to 100%;
(ii) a sequence encoding a portion of a PKS of the invention, wherein the sequence has a sequence identity to the full-length sequence of any of SEQ ID NOs. 2 to 10 or SEQ ID NOs. 21 to 33 from at least 85%, 90%, 95%, 96%, 97%, 98%, 98.5%, 99%, or 99.5% to 100%;
(iii) a sequence completely complementary to any nucleic acid sequence of (i) or (ii); or
(iv) a sequence encoding a polypeptide according to any of SEQ ID NOs. 11 to 19 or SEQ ID NOs. 34 to 46.

14. A vector comprising at least one nucleic acid according to claim 13.

15. A host cell comprising at least one nucleic acid according to claim 13.

16. A method for the preparation of a compound of formula (I), the method comprising the steps of:
(a) culturing a host cell of claim 15; and
(b) separating and retaining the compound from the culture broth.

17. A method for treating as patient susceptible to or suffering from a bacterial infection, comprising administering to the patient an effective amount of a compound of claim 1.

18. The method of claim 18 wherein the patient is suffering from a Gram-positive bacterial infection.


SEQUENCE LISTING (1 sequence)

SEQ ID NO: 1 - DNA, 37365 bp, Angiococcus disciformis
  misc_feature (20206)..(20215): n is a, c, g, or t
tcagctggcccggaacgtcatcggcatgctgaccatcccgtgcagcaggtagcccggcacgggctccagctcggccccctccaccatccgcagctccggcatgcggcgcaggatggccgggatggcgatacgcgcctccaggcggcccagcggagcccccatgcagtagtgggcgccgtggccgaacgagacctgacgcgccgccttgcggctcacgtccaggcgctccggctcggggaagacgtcgggatcgtggttggccgcgccgagccacacgatgacgcgctggccccgcttgaggagcttgccgccgacctccgtgtcgtgccgcacccagcggggcgcggcgaggaccggtgagtcgtagcggagcagctcctcgatggcccccggcatctggtccgggttcgcgaggaagcgcgccatctggtccgggtgcttcaggagcgcgcggatggcgttgccaatcatgtgggtggtggagatgttgccggcgatgagcagcagcatgcagaaggcgaccaggtcgagctcctccagcctctcgccgtcgatctccgccgccaggaggctgctgacgaggtcgtccgagggacggcggcggcgctcggcgatctgctgccgcacgtagtcctccatcggcgtcagcgcctccgacaggagcctcaccccgttctccggatcgagcaccagggcattggcccggacgcccacctcgtccgaccagcgccggaaggactcctggtccgacgccggcacgcccagcatctcggcgatcaccatcgtgggcagggggccggcgatgtccgccacgagctcggtcctccccgtgccgaccacggcgtcaatcagctgattgacgatggcggtgatccggtcctgcatgcgggccaccgccgaggccgtgaaggccctcatggccaggttgcgcagctgggtatggcggggcgggtcaaaggcattcaggctgaagaggcggcgccactcggcggggggcgccccgccatcatcgatggtcgccgtcgggaagttcacgtggtcggagaggatggccgtcacgtccgcgtggcggtagacggaccagaggttggccccgtcatcgaatgccaccgggtgatgcctccgcatctcggcgaagaacggataggggttcaacagctccggaccggccgggaacgtacgcatggtggctcttccttccttgtgaactggaaacgggtttgcccacggcttccacctggtgaaaaccaggggcgggcttctcgcgcgtggatggcgggagaatctcgggcgtctgatgaaacctgtcggtcacggcgcggtgatggacagggcccctatggacggctgtatagataagcaatagatggcgcgcaagggggcgagtcaagtctcgcgccgccgtatgaagtcaaaacagattgcccctgtctcatgacagccgtgtggacagacacacgcgacagtacgcgcgaggccacgagcctgggcccgttcggcacttctcacgacactggcgacgagcgcagcgaacacacggggagtgaacagcctccgcgggggcgggctgggccattccctcgcccgtgacggggcggccccgggtccgcgcccggtccgccaggcgaccgccacagcatccgcatccgcaaccgcaagaggcggaatttttacattgtgcagtacttcacgtttttacttgacccgccgcattaacccgaaataagcatgaagccagaataatgagggggcgttcatgtttgtgatgagtgggatctactgagaaagcattcggtgtcacaggggagacaccgatgtcgcagtcattgccactggaagcagttggcgccgcgacaccaccccgaagccagttccagcagtccgtccagcgcgctccgcgcggctgtctgccattggtttgttcagaaatccctgccttctctaccgacacgtcgggtgacgtgctcgcccgcgcggacgtgtaacgactggaagccatgactggaatcatcgacaagctgagccggctcgatgccgcgagcaaggacctcctggcggagaagctgcgccaggccaaggacaagcacctggcgcgaggggcgcacgccgagcccatcgcggtggttggcatggggtgccgctttcccggtggcgccagctcgcccgagcgcttctggcagttgctgatgcaggggcggagcggcgtcggccgcgtccccgcggatcgcttccccatcgaggactactacgaccccgacccgagcgcgccgggcgccatcctcagcgaccggggcgccttcgtggagggtatcgaccgcttcgacgctggcttcttcggcgtgtccccccgcgaggcacggcgcctggaccctcagcaccgcctcctcctggaggtcacctgggaggcgctggagaacgcgggcctgccccagtcggcgctccgcgagctcgttggcggcgtgttcatcgggttgatgggcaacgactacttctccaccgtccaggagaaccccgccgacatcgacgcctacaccagcacgggcacgcactacagcttcgcggccaaccggctgtcgttccatctggacttcaagggcccgagcatggccatcgactcggcctgctcgtcgtcgctggtggccgtccaccaggcctgccagagcctccggacccgggactgcgacttcgcggtggccggcggcgtcaacctcgtcctctcccccgtcctgagcatctcgtactcgaagtgggggatgctcagccccgacggacagtgcaagaccttcgacgcctccgcgaacggcttcgtgcgcggcgagggcgccggcgtggtcgtcctcaagcggctggaggatgccgtggcggccggggaccccatcctcgccgtcgtgaggggctcggccgtcaaccaggatggcgccaccagcgtcatcaccgcgcccaacgggctcgcccagcaggccgtcatccgccgcgccctgaaggtggcgggcgtggagcccgccgccatcggttacgtggagacccacgggacggggacgtcgctgggggaccccatcgaggtggaggccctcaacgagacctacgggcaggagccgggcggccgcccgtgcgtgctcggggccgtcaagacgaacgtggggcacctcgagggagcggccggcatcgccggcttcatcaaggcggtcctcgccgtacagcgcgggcgaatccccccgaacctccacttccacgagctgaacccgctgatccggctcgacggctcgcggctgtgtctggccacggaggcccgggagtgggccgtggcgagccaggagcgcttcgccgccatcagctcgttcggcgccggaggcaccaacgcccacgtcgtcatccagggcccccccgccgcgcgggcgcccgcggccaccgggaaggcgcccgccttcgtctgcctgtcggcgaagagcgacgcctccctgaagcagcaggccgcgaggctgcgcgcctggctcgggcaggacggccgggagtggagcgtcgccgacatcgcctccacgagcctgcggcgcaggaaccacttcgaccaccgggcggccttcaccgccgactcgccggagtcgctcggtgagggcctggacgccttcctgtccggccgggccagcccgcgctggtccgccggcagcggcaccctgaacgcccagcggccggtgttcgtgttctccggccagggctcgcagtggcagggcatgggccgggagctgctcacccagagcccgcgcttcgcggcggagatcgccgccatcgacgcgcggttcatcccgctggccggctggtccatcgccgaggtcctcgcgcgggacggcgacggggcgcggctgaacgccacggaggtggcgcagccctgcctgttcgccctgcagctggggctcgtcgccgcgctggacgagctgggcatccggcccgccgcggtcgtcgggcacagcctcggcgagctcgccgcggcgtgcgtggccggcctcatggaccgcgaggccgccacggccctcgtgtaccaccgcgcgaagctcatgcagcggctgaccggcgcggggaagatggccgccgtcgggctctcccgcgaggaggccgaggcacgcctcgcggcggagccccggctggcgctcgcggcggtgaacgggccccgctcggtcgtcgtggccggtgagccggaggccgtggacgccttcgtgcgggcgctgcgcgacgagggagtcttcacgcggacgctgccggtcgactacgccttccacagcccccagacggacccgctcctggccgagctgcggcgggccgtcgacggcatcttcggcgcgggcgcgaggatggccgccccccggctgccgatggtgtccacggtgagcggcgccgagctcgatgcctccgtcgaccacgccacctactgggcccggaacatgcggcagacggtgtgcttcgaggaggcggtgggccagctgctggagcagcggcacgacgccttcctggaggttggcgcgcacccggtcctgaacgtcgacctgcgggagtcgttccgcgcgcgggaggtgacggcgcgggccgtcgccaccctccggagggagcagccccaggccctctgcctgcgggcggtgcccgccgcgctctacgccgccggcatggcccccgaggcgctccccggccaggaccagggcaccctcgtcccactcccgaactacgcgtgggaccaccgccgctactggattcactccgcctcgcgggccgaggcgggctgggtctacgacctcgcctgggacgcggtgccggcacctgcgccgggcgcccaccggcccgcgtcctggctcgtcctgggcgcctccggcgggctgccggaggcgctggccgcccggctgcgcgccacgcccgacctggtgcgccacgtgagcctggaggcgctgcgctccgtgcccgtgcccccccacgggctggtggacgcggagcggctcgagcgctggttcacggacacgctccaggtggacctggcggagttcgacttcctcgtctacgtgcccgcgtgcggcgcccccgaggacgcgagcgccgagcgccaggtcagcgaggactgcgccatcctggtgagcctggcccaggccctggagaggcggcccggggcccggggcccggcgctctgggtcgtcacgagcggcgcgcaggccgtcgcgccgcgggagcccgtcaatcccttcgccgcgcccgtgtggggcctcggccgggtgctcgccctggaggccccgcgaggcctgggcggtctcgtcgacctgccgctcgcgtggtcggagcaggacgtgggctgcctgctcgaggcgctgcgccaccccggcgaggaggaccagctcgcgctgcgctcgggccagcgccatgccctccggctgcgccgccgggcgggccgcgccaccgacgcgggcgcggcctggcaggtcgaggactccggcgtctacctcatcaccggtgggacgcggggcctggggctgctcgtggcgaagcggctggccgagcgcggcgcccgtcacctggcgctgctgggccgccgggcgctgccgccccgctcgcggtgggcgacgctggccgcggacgacgccttcttcccctccgtgcaggccatccaggcgctggaggcccagggcgtcaccgtcacgctccacgccgccgacgtcagcgactcccacgcgatgcgggacgtgttcgcggagctcgagcggcagcagcgccccctcaagggcgtcgtgcacgcggcgggtatctcccgggaggtccccctggcctccatcgacgaggcgctgctggaggagaccctggcacccaaggtcgcgggcacctggtggcttcaccagctctcgctgcggcacccgctggacttcttcgtcgtcttctcttcgggggcctccgtctggggctccgcgggcctggcgccgtacgccgccgcgaacgagttcctgaacgccgtggcggcccaccgccgcgcctccggcctgcccgccctcagcgtctgctggggcatgtgggacgcggacggcatggccaccgacgagatgcgggagaactggacgcgcctgggcgtcaaggcgatggcccccgagcgcgccctcaacgcgctggaggagctggtggccgcggacgccaccctcgccaccgtggccgacgtgaagtgggcccgcttccggcccgtctacgagctgaagcgcaagcgtccgctgctggcggcgctcgaggacggcgcggcggcaccgccgcggcccgaggcggccggggcgccccaggccacctccctcaaggcggacctccaggcgctgccgcgggagcagcagcgcgagcggctgctggccaccctccgcgacacggccgcgctcgtgctcggcgccgagccgggtgccgccatcaacccggacgaggggctgttctcgatgggcttcgactccgtcacggccgtcgagttcgcggcgcacctgagccgcctgttcgagaagcgctacgccgcgacgctggtcttcgaccacccctccctcaagcagatccgcgacttcctcatgcaggactgcttcggctggacggccgagccgtcccccgagcccgaggcgggtgacggccaggacctcgacgcgctcctcgccttcgtcgagggcgtggacgaggccgaggccgagcggctgctctccgccgggcagccccggtcgaactgagccctccagcgacgcgcgagacgtgccatgacctccgtcgacgaacgcatcaagaatctctccccgcagaagcgcgccctgctcgagcagcggatgaacgccgccgccggcgccacccgggacgcggagcccatcgccatcatcgggattggctgccgctacccggggggcgtgaaggacctgaagagcttcgcccagctgctgcgcgacggcgccgacgccaccggggacatccccgcggaccgctgggacgtggaggcccactactcgcccgacccgggcacccccgggaagatgcgcacccggcgcggcggcttcctgcccgacgtgaagaccttcgacgcggacttcttcggcatctctccgagagaggcggagagcatggacccccagcagcggctgctgctggaggtgtcctacgaggccctcgaggacgcggggattcccaccgcctcgctgagcgggagcaccaccggcgtctacatcggtatcaccctggcggagtacgccgaccggctgccggacgacccccgcctcatcgacgtctactcggggacgggcacctacctgaacgtggccgccgggcgcctgtcctacgtctacggcctccagggcccctgcatggccgtggacgcggcgtgcgcctcctcgctggtgacggtccacctggcggcccaggccctgcggcaccgcgagtgcggcctggcgctggcgggcggcgtgagcctgatgctgtcgccggagaccaccatctacctgtcgcagacgggcgccctggcccccgacggccgctgcaagacgttcgacgctcgcgccaacggctacggccgcgccgaggggtgcggcatcctcgtcctcaagcggctgtctgatgcgctgcgcgacaaggaccgcatccacgcgctcatccgcggcacggcggtcagccaggacggcgcgtccagcggcctgacggtgcccaatggccgcgcccagcagaaggtcatccagtccgcgctcaaggacgcggcggtgcggagcgaggacgtctcgtacgtcgaggcccacggcacgggcacgccgctcggggacccgatagagctcaacgccctccaggccgtctacgggcagcgcgaggcggactggccgctccacgtcggctccctgaagacgaacttcgggcacgccgaggccgcggcgggcgtgggcggcatcatcaagacggtggtggcgctccaggagaggcagatccccgcccacctccacttcgagaagctcaacccgctcatcaccatcgaccccacgcgcgtcgtcatccccacccggcccacgccgtggcaaacgcggggggagcggaagcggctcgccggcgtcagctcgttcgggttcagcggcgtgattgcccacgccgtcctggaggaggcgcccgcgccggaagcggccgtcgagcagccggcgatgcccgccggggagcggccgctgcacgtcctgccgctcaccgcgaagcacccccgggcgctgagcgagctcatctcggcctaccggcacaccgtcggcgcgctcgacgcgggccggctcgccgacctgggctataccgcgagccggaagcgcggccaccacgcgcaccgcgcctggttcgccttccgcgacaaggaggagctgctccagcagctcgaggccgcgacccggggccacgagctgcccggcgccgtgctccagcggcaggcgcgcgacgacgtcaaggtcgtcatggtgttccccggccagggctcccagcgggtcggcatgggccagcagttgatgcggacctccccggtgttccgcgaggccatccacgagctggaccgcgcgatgcggccctgggcggacttctcgctgctggaggtgctcggcgcccgcgggggcgaggaccccttcacccgcgtctccatcgtccagccgaccctcttcgccgtccaggtggccctggccaggcagtgggaggcctggggcgtcaggccgcacgcggtcatcggtcacagcatgggcgaggtcgcggccgcgtgcgtcgcgggcgcgctcagcatggaggacgccgccgcggtcatctgccgccgcagccagctcgtccaggccaccagcggccagggcgccatggcgctcgtcgagctgagccccgccgaggtcgaggccgccatcgcgccccacggggaccgcgtcagcatcgccgcgtacaacgggccctccaccgtcctgctgtcgggcgagccggccgccgtcgacgaggtgctcaccgccctccaggagcgcggggcgttctgccgccgcgtcaaggtggactacgcctcgcactgcgcgcagatggacccgctgctgggccgcatccggagcgagctggcccacgtgaagccgcgcaagggcggcgtgcccatctattcgaccgcgcgcggcgaggtcatcgacggctcgcggatggacgcgggctactgggcggacaacctgcggcgccccgtcctcttccacgcgcaggtcaaggcgctcctcgcggacggcatgcacgcgctgctggagatcagcccgcacccggtgctcctgccgtcgatgcagaagacgatagacgaggccggcggacagcagctcgtgctgcccagcctgcgcagggacgaggacgagccgcggagcctcgcgacgtcgctgggcgccctcttcggcctcggccacccggtggacttctccgccttcaccccgaggtccggccggctcgtcgagacgccgcgctacccctggcagcggcgcgagttctggctgacgcggaaggcgtcgaagcgccggcacgcgctgggcggcgcggagggacatcccctcatcggtggcgccttccgcccggccgtgggcccggagtcctactactgggagttcgacgtcgacgaggagcgcctcgccttcatcgccgaccaccgcgtgcgcggcatgacggtgctgccggggacggcgtacctggacctcgcgctctcggcggcgcgggccgccttcggcgacagggccttccccctcagccaggtctcgttccgggaggcgctgaccctgctgcccgagcagccgcggcgggtgcagctctcgctcgtccgggaggcggagggcgcgttctccgtctgcgtctccagccgcgacgcggcgtccccggccaccggggagtggaccctccacgcgacgatgcgactggacgtcgaggccccggccacgggaggcgctcgcgccgagggacaggtgacgccccccgcgggcgccgagctccgggacgccaggacgcactaccgcctcatgaaggagggcggcctcgcctacggcccgcgcttccaggggctggcggaggtccactgcggccagcacgaggccctggggcgcctgcgcgcgctgccggagctcgggctcgacggcgcggaccacgtcttccaccccgccctctgggacgccgcgcaccaggcgctggcggccctgctcggccgccaggacggccacgcctccggcccgtccacctggattccgcgcggcatcgagcgggtctcggtgtccggcaccgtcccggcccgggaggcgctgtgggtccgcgccagcgcgcgggtggacgcggacggcgccacggcgcggggagacctccaggtcctggatgacgccgggcgggtgctggtcgagacgcgcgggctgacgctgctccggcgcgacgcggacaacctcgacgacgtccgccagtggttcttccggccggtgctccgcgaggccccgctccgggaggaagcccccgcggacgggacgtggctcgtcctcggcgcgcccgggcccctgcatgaccgggtggtggcggagctgcgcgggcgcggcctcgacaccgtgcaggcgttcccggccaatacctgggaggagcaggacgcggcgcgcttccacctctcccccgcggaccccgagcacctgcgccggctgatggagcgggtcagcgccgaccgtccgctgcgcggcctcatcaacctccagccgtaccaggtggcgcgcggcgacgcggcctcctcggcgccggagggcgtgctgtccgccctctacatggaggtgctcgaccaggcccgggcgctccagggcgtgagccggcgggagcggccgaagctggtgctcgtctcgggcgtctcccagggcgcggaggccgagcccgacttggtcgccgccccgctccagggcctgggccgcgtcatcgcggccgagctgccccggctccactgcgtccaggtccagctcggaggtcaggagcccgaggtggaggcgcgcgacctcgtgcgagagctgtacgccaacgacggcgaggacgaggtcgtgctgcgcgggggcgctcgccacgtgcgccggctcgagcggctcgtccccggcgcggccccggcggcgcccatccacgcggacggaacctacctgctcacgggcggcctgggcggcctggggctggccttcgcgagctggctcgcggaccgcggcgcgcggcacctggccctggtcagccggcgcccgccctcgggggaggcccgcgcgcggctcgacgcgctccgcgcccggggcgtcgaggtgctcgtgcgcgaggtggacctgtcccgggaggactccgtcgccgccctcttccgggagctggacgagcgcatgccgcccctgcggggcgtcctccacctggcgggcgtcctggacgacgcgacgctgttccagcagaacgagcagcgcctcgcgaccgtgcgcggccccaaggtcaccgggacgcggctcctgcacgcgcacacccagcaccggaagctggacttcttcgtcctcttctcggcgggcgccaccctcctcggctcgccgggccaggcgaactacgccgcggtgaacgcgttcatggatgcgttcgccccgcaccgccagcggctggggctgcccgcgctctccatcaactggggcgtctgggacgaggtcggcctcgccgcggcgagcgcgcagcgaggccagcgcctgcgggagcaggggctgttccccatcccccccgcgcagggcctccaggccttcgagctggcgctgtcgctcgcgggcgccaacgtcgccatcctgcccatccgcctcgcccgctggctgcgcgagcacccggacctggcgggcgcgtcgctgttcaaggagctggtccgcggactggacgactccgaggcggaggccgaggcgcccatcctggagcagctgcgcgccacggtggacccgcatgagcagcgcgagtgcctgcgccgctgggtcgggcgccagctcgcggcggtgctgcgaagggaccccgcgtccatgcagcgcgagcagaacttcagcgccatgggcgtcgactcgctgatggcgctcgagttccgcaaccggctggagcgctcgctccacgtccggctgccgccgacgatggtcttcaaccatccgagcatcgaggcgctctgcgggttcctcgagccgaagctgggcatgggcgccccctccgctccggcgcagccggagcccgtgcctgctcccgccgcgcccgtgcgcgtcgacgagaaggacggcgccgcgctgctcggggccctggcccggctgaagaagctcacccagcaccaggcgtagttcgagccacctatcaatcctctggagacgcaggccgatgtcgatgttggatgagctcagggcagtgctggacagtctgtcgccggaacagcgggcgacgctcgcggagctgctgagcgctgacgcgccccggaaggccgacgagccgatcgccatcgtcggcatggccggacgcttccccggagcacgcgacctggcggagttctggagcgtgctcgccgaggggcgggacgccgtctccgagattccaccggagcgctggaacgcggacgccctctatgacccggacaaggacgtcccgggcaagctggccacccgctggggcggcttcgtgaaggacgtcgacctgttcgacgcggagttcttcggcatcactccgcgcgaggcggcggccatggacccgcagcagcgcctgttcctcgagacggcctgggaggccctcgaggacgcggggctgcccgcggcctccctcgcccggacgcagacgggcgtcttcgtcggagtctccaactccgactacatgtggcaccagttcccccggctcgacaaggtggacgcgtacgcgagctccgggtccgcgcactgcatcatcgccaaccggctgtcctacctgctggacctcaccggccccagcgtggcggtggacaccgcgtgctcctcgtcgctggtgtcggtgcacctggcctgccagagcctgcgcaaccgggagtgcgaggcggcgattgccggcggagtgaacgtcgtcctgacgccgcacctgggggtgagcctctccaagtgggggatgatggcgtcggacggccgctgcaagacgttcgacgcccgcgcggacggcttcgtccggggcgagggcagcggcgtgctcgtgctcaagcggctggcggacgccgagaaggcgggggaccgcgtcctggccgtcatcctcggcagcgccgtcaaccaggacgggaagacccacggcctgacggcgccgaacggcgccgcccagcaggccgtcatccggcaggcgctggcccgcgcggggctgtctccctcgcaggtgtccttcctggagacccacgggacggggacgtccctgggcgaccccatcgaggtcgaggccatcaacgaggtgttcggctccagggtcgagcgctgcgtgctgggcgcggtgaagaccaacatcgggcacctggagccggcggcaggtatcgccggcctcatcaaggtcgtgctcgcgctgcgccacgggctcatcccgcgcaacctgcactaccagtccatgaacccgcacctccagctcgctccgggcatcgagctggcgagcgaggcccggccgtggaagcgcgacgccaggccccgcttcgccggggtgagcgccttcagcttcggcggcaccaacgcgcacatcgtcctcggagacgcgcccgcctacgcgccgccggccgagtccgcgcggcccgccctgcccaccctcctccccctctccgcgagccaggagggggcgcggagccagatggccgcgctccacgccgggctgctcgccaccgcggcccccgagctcacggtggacaccgtggcgggactgcgcgcgctgcaccgggaccacctggacctgcgcctggccgtcgtcgcctcggaccgcgcgacggccgccgaggccctgaggtgcttcgccgaggggcggccccacgcgggcctggtccacgggcgccgccagccgaacctgcggcccgtcttcatctacaccggccagggcaaccagtggccgggcatgggccgcgagctgatgcgcaccgagccggtgttcagggccgcggtggaggaggcggacgccgcgttccagcgcctcacggggcggagccacgccgccagcttccgcgaggacgcgtccgacgcggcgtcgctggagcgcaccgacgccgcccagccggccatcttcctcctgcagtacgcgctcacgaagacgctggagcactggggcctgcgtcccggcgcggtcgcgggccacagcgtcggggaggtcgccgccgcctgggccgcgggcatgctggacctggacgacgcggtccgggtggtccaccaccgctcgcggctcatgcagcgcgccaccggccagggggcgatggcctccgtcgcgctgtcgccacgggaggcgcggcccctcctggaggggcgcccctcgctggccatcgcggcgcacaacagccccggggaggtggtgctctcgggtgaccccgccgagctcgaggccgtgctccgcgagctggaggggcgcggcgtccgctgcaggaagctccccgtcaactacgccttccacggcccgcagatggacccgctccgcgacgagctgctgcgggagctgtccgtcctccggccgaaggcgccgcgcctggcgttctactcgacggtgacgggccagcgcgagcggcaggccgtggccgacgcgagccactgggtccgcaacatgagggaccccgtcgcgttccgcgaggtcgtcgagcggctcgcgaccgacagcccgagcttcttcctcgagctcggcccgcacccggccctcgtgccctcggtgatggacacgctggcgcacctccggctcgaggcgcgcacggcgcactgcctgcggcgcaaccgccccgaccggcacgagctgctgtccgccgtggcctcgctctacgtccacggcatcgacccggactggtcgcgactggtgcgccccccggaccgccggtgggcgctgcccgcctacccctggcagcgccagcgcttctggctggaagcggcgccccccccggacgccacctggagcgcgccgagcgcgcggcccgccggccacctcgtctaccggaccgagtggctggccgacgcggcctccgcctccggggacaccgcgcggccgaagggccacctcgtgttctccgacggcagcgccctggccgaggccctccgcgccgagctcgaggcggccggcgcgcgattcgggtgggtgagccgcggccaggacttcgccgcggacggcacgcgctgcaccgtggacccggagaaccccgagcacctcgagcggctcttcggccacctcgcggccgagtccttcgagttcgaccacgtcctgctgctgttcaccccggtggcggacgaggcgcggccggacggcgcggcgccgtggttcgacacggccgtgcgccccgtgcacgtcctgcgctgcctgacccaggccctgctcacgaccgagcgccggccgaggatggcggtcatcacccgggcggcgctgagcgcgggcggcgacaccgcggcggtgccccagcacacggcgctctggggcttcgggcgcgcgctggcgctggaggagccggggctccgcctggcgctgctggaccttccggcggtgcccgacggcctcgtggaggcccggcacgtcctggcgcgcctcgccagcccgacgcccgagcgccagtcggcgtggcgggacgggcggtggcgcgtcccccgcctcatgccccacgaggccgggacgggcacgtccgcgccgcccgtcatccagggcgagggcacctacctcatcaccgggggcctcggctcgctggggctcgccaccgcccgccggctcgtccagcgcggcgcgaggagcgtggcgctcgtctcgcgccgcccgcccaccgccgcgcagcaggccgagctcgcgcgcctggaggaggcgggtgcccgggtggtgacggtgcagggcgacctggcggacctggcggaggccacgcgcgtcttcgagacgctcgcggcgtcggcgcctccggtccggggggtgattcacgcggccggcgtgctggaggacggagtgctcgggagcgcgacgaaggcgagcttcgaccgcgtgctgcggcccaaggtcgccgggagctggaacctccaccagctcacgcagtccctcccgctggacttcttcgtgctgttctcctccgccaccgcgacgctgggcgccgcgggccagacgaactacgccgccgccaacgccttcctccagggcctcgcggagcaccgccgcgcccgcggccagcacgccctctgcctgcactggggcgcctggcgcgagagcaccatggcctccggcgccgtggtgacgaagctgaaggcgctgggcttcgacaccctggcgcccgaggacgcgctggacgcgctggagcgcgtgctcctcgaaggggcgacggaggcgaccatcatcgccgccgactgggaggtcttcgcccagcggctcccggacgagagcctgggccggttcctcgagcccgtgctgcgcgcgagcggggccccgccggccgtgaagcccgaggccgaccccatcgtctcacgcctcgccggcgcgccggcgagccagcgccagaagctgctctacggccacgtccgcgcggtggtgctgtccaccatggggatggacgcccgcaccgaggtccgcccggaccaggggctcatcgagctggggctggactcgctgctcgcggtgaacatcgccaaccggctctcgcgcgacctggcccgccagctgcccaagacgctcatctacgaccacccgacgctgcacgccctgacggaccacctgctgggcgtgctcttccccgagcccgcccaggccagcgtccggcccgccgcctccggcgacgcgccctcctacgaggacatcgccaacgcgagcgcggacaccatccagagcctcctcgacgccgagctcgcgagcctcctctcctcccgctgagcccctccgcgggcgtgcgccggacgcggtccccgcgggggtccgcgcccgcgcgcccggtgcggctccttcgcacagaaagcacttccgccgtggatacgaacgccctccttgcgcagaacaagaaagccctcctggccatcatggagctcaaggcccagctcgcgcgggccgagtcgcggcagcgcgcgcccatcgccatcgtcggcatggcctgccgcttcccgggtggctgcgccacgcccgagcagttctgggagctgctgcgcgagggccgcgacgcggtcaccgaggcgttcccctcgcggtggccggagtcgctctacgacgccaaccccgaggcgcccggcaggatcatggcgcggggcctgggccaggtggagggcgtggccgacttcgagccggagttcttccacgtctcgccgcgcgaggccgcgagcatggacccgcagcaccggctgctgctcacggtcgcctgggaggcgtgcgaggacgccgggctcacccgggagctgctgcacggcagccgcaccggaatcttcgtcggagcgggcaacaacgactacggcgacttcgagctgcgcaaccccgacctgggcgcggtcagcgcctacgcgggcacgggcaacgccacctcgctggtcgcggggcggctctccttcttcctcgggacgcagggccccgcgctcgtggtcgacacggcgtgctcgtcctccctcgtcgccatccaccaggcgtgcaccagcctgcgcctgggtgagtcggagctcgcgctcgctggcggcgtcaacctggtcctccgccccaacggcagcgtgttcctctcgcgcttcggcgcgctctcgcccaccggccggtgccgggccttcgccgaggcggccgacgggtacgtgcgcgcggacggctgcggcgtcatcgtgctcaagcgcctggaggacgccctgcgggacggggaccgggtgctggccgtcatccggggctcggccatcaaccacgacggccgcagcggcgggctcaccgtccccaacggggcggcgcagcgggagctgctcctggccgccctgaagaacgccggggtggagccgcggagcatcggctacctggaggcccacggcacgggcacgccgctcggcgacccgatagagatgggcgccatcgccgacgtcttcggcggggacgcgcggcgcgagtccctgcgggtgggctcggtgaagagcaacatcgggcacaccgaggcggcggcgggcatcgccggcatcatcaaggccgtcctcagcctccagcaccgggaaatccccgcccacctccacttcgacaagcccagctcgcgcatcgactggagcatccccgtcgaggtccccacgagcacgcggccctggagcggggacggcccccggctggcgggcgtgagctcgttcggcttcagcggcacgaacgcacacgtcatcctggaggaggcgccgcgccaggaggcccggcactcgccccggctcgggacgcaggtgctctgcctgtcggcccgcaccgagggctccctcacgcggctggccgcggcctggagggagcagctccggcggagggattcggcgcccctggccgacctgtgccacgccgccgcgcggcgccgctcccaccaccgcgcccggctggccgtcctcggcgacagcgccgacgccatcgcccaggcgctggagtccttcgccgccgggacgccccaccccgccgtggtgaggggcgacgcggtgctcgacgggatgtcccgcacggccttcgtcttctccgggcagggcagccagtggctcggcatgggacaggagctgctcgccagcgagcccctcttccagcagagcctgcgcgaaatggacgggccgttccgcgccgagttcggctggtccctcgaagaggcgctgcgggccggggaggccgagtcccagctcgagcggacgcgcgtcgcccagccggtcatcgtggccgtccagctggcgctgagcgacctctggcgggcgtggggcgtgcaccccgacgtgacgctggggcacagcctcggggagatctccgcggcccacgtcgccggcgtgctcgggcggcacgaggccctgcgcctggcctgcatccgcggccggttcatggatggggccaccgggcgcgggaagatggccgagctcgcctgctccgaggagcaggcgcggcgctacctccagggcttcgaggccgaggtcgccatcgccgccctcaatggcgccatgtcgacggtgctcgcgggcgcgccgggcgccctggaggctgtcctccagaaggtcgccagcgacggcatccagcaccgcctgctgcgcgtggactacgccttccactcgccgcagatggccgagttcgcggcgcccttccgcggcgcgctgggcgacatgaagccccgcggcgccgagcgggacctctacagcagcgtgctcggccgcgcggtgcgcggcgaggcgctgacggccgactactgggtgcggaacatgacggacaccgtgcgcttccacgaggcgctcgtggcgctcctgcgcgacggctgccgcgccgtggtggaggtctccccccaccccgtcctgctctccgccatccaggacaccctgagccgcgagggcgtcgacggcgtcgccgcgtactcgctccggcgcggcaacccggagcgcgccaccctgctgtcgaacctcggcgccctgtacgccgccggccactccgtggactggtccgcgctctaccccgggctggtggaccacgtggccctgccgacctacccctggcacgcccggcgcttctggacgaccgtccagggcccggaggccaccaccggcgcggcccccgccgtcaccgccccggccccgaacgaggccgcccccgccgccgtcacctcgaagctgtgggacgagctcgtcaagctggcggccgtcgcgcgcaggccgcgcctggagcgccatgtccgcgagcagctcacgggcatcctcaagctggactccggggacatcccctcgaagcgcacgccgctgagcgagctgggcatggactccgtcggcgccgtcgcgctgtccaaggcgctggccctcacccacgagcaccggttccctcccaccgtgctcttcgagcaccccacgattgaagcgctgaccgggttcctcgccgaggagttcgagcggctccnnnnnnnnnnccgcccccccccccccccggccccccgccgctccggaggccgccgcgctcccggtgcccgactcggagctcgccatcctggagaagctcgccgccctcgagaaggagtttgcctgacatgtccacaccggagaatcccacgggcgaccgcgcactcctccaccgggcgctggccgccatggagaagatgaagggccgcatccagcagctcgagggcgtgacgaccgagcccatcgccatcatcggcatgggctgccgcttccccggcggcgtccacggccccgacgccttctggcggttcctcgcggacggcgtcaacggcatccgtgacacgccgaaggaccgctgggacaccgagcgctacctcgacgccgagcagggcacgccggggaagatgtacacgggccacggcggcttcctggaccgcgtggacgggttcgacgccgcgttcttcggcatctcgccccgcgaggcgctgagcatggacccgcagcagcgcctcttcctggagacgtgctgggaggccatcgagaacgcgggcctgtccgcggacgtgctcgagggcagccagacgggcgtctacgcgggcatcgtcaaccaggactacgccatgatgcagatgaaggacaccgcgcatggcgagatggacacctacttcgccaccggacacccgcactgcgtggtgagcggccgcgtgtcctaccagttcggcctccgcggccccacgctgtccgtggacaccgcgtgctcgtcgtcgctggtggccctgcacctggcctgccagagcctgcgcctggggagctgcgacgtggccctggctggcggcatcaacctgctgctgatgccggagatctacatgatccgctgccaggcgcgcatgctgtcgcccagcgggcggtgcgccacgttcgacgcgtcggcggacggatacatccctggcgagggcgtgggcgtcgtcgtgctgaagcggctgcgagacgcggtccgggatggggaccgcgtcgacgccgtcatccgcggcacgcagatcaaccacgacggccgcagcagcgggctgaccgtgccgagcggggccgcgcagcgggagctgctgcgcgccacgctgcgcgacgcccgcctggagccccagcgcgtcggctacctcgaggcgcacggcaccggcacgaagctgggggaccccatcgagatggccgccatccaggacgtctactgccaggaccgcgcgcccggaaacccgctggtcatcggctcggtgaagaccaacatcggccacctggagtccgccgccggcatcagcggcctcatcaaggccgtcctcaccctcaagcacggcgccatcccgcgcaacctccacttctccgagctcaacccgcacatcacgctgcacccggcgacccggattcccaccgagctcaccccgtggccggcgggcagcggcccgagggtcgccgccgtcagctcgttcggcatgagcggcaccaacgcccacgccatcctggaggaggcccccgcgcgcgaggccgccagcgcccaggcgcccctccgcgtcacctcccggccggcgacgccgcggctcatcccgctcagcgcccaccaccccgaggtcctccaggcccatgccgccgggctgaaggaacacctggacgcccacccggacgtcaccttcgacgacctggcgcacacgctgcgccggcgcaccctccagcgccaccggctcgccctgcccgccagctcgctggccgaggtgaaggagcacctggcggcgttcacggccggcgggcaggacccccgcgtcctccagacggaggacctcttcttcaccgacccgaaggtcgtcttcgtcttcccgggccagggctcgcagtgggtgcggatggggctccagctcatggagcaggagccggagttcgccgcggagctgcgcgaggtggatgcgtggatccagaagtacgccggctggtccctgctggacgagctccgcgccgacgcgagcgcgtcccggctgagccaggtcgacgtcgtgcagccctgcatcttcgccgtgcaggtcgccctcgcccggctgtggcgctcatggggcgtcgtccccaacgccgtcatcggtcacagcatgggtgaggtggcggcggcctgcgtcgccggcatcctgtcgctcgaggacgcggtgtgcgtcatcacccgccgcagtcagatcgcgaagcgcaccagcggcaacggcgccatggcgctggtggagctgacgcgcgagcaggccgagcgggccatcgccgcgcaccaggacgtggtcgcggtcgcggcccagaacgggccccacaccgtcctcctctccggcacgccgggccccatcgagcagatcctccgcgagctgcagcagcaggacgtcttctgccgcgccatcaaggtcgactacgcctcccactccccgcagatggatccgctccggcccgagctccagcgggcgctctccggcgtgaagccccgggcggccaccgtcgccttctactcgacggtgacgggccaggtggaggacggcacgtcgcacgacgcgaactactgggtgaacaacctgcgcgaccccgtcctcttctggccgggcatgcagaagctcgtcgaggacggacacaacatcttcctggagctcagcccccaccccgtcctcatccccgccatctcccagggcctgcagcacctgggcgcccgggcgctgagcctcccgtcgctccaccgcgagaagccggaggccgagtgcctgttcgagtcgcgcgcccggctcttctgcgccggcggcaggctggacctcgcgaaggaggacgtcccgggcgcccgggtcctcgccctgcccggccacccctggcgtgagcagcgcttctggtacacgcccagcgccccctcggcgcacccgcaggcccgcggcggcagccggctgctgccccgcatgtggacggcgggctccggggagcggcgcgtcttcgagggcgacgtcggccccggcgtcatgccctcgctggcggaccaccgcgtcaatgggatgcagctcttccccggcgccggctatgtcgagctggtgctcgaggccgccgaggcgctcgggctcgagggccggacgcacatcgactcgctcgacatccgcgaggggctggtgctccacgagggcgcggcgcgcgtcgtccaggtggagctgacgcccgtggccgcgcgctccttcgggttcgtcatcagcagccgcgaggccgacggcgccgcctggacgcgccatgccgacggcgtggtgacgcgcgaggaggccggggcggcgccgggcgcggagcccggcgcgctggaggccttgcgcgcgcggctcgcctcgcccctgtcggcggaggaccactacctccgcacccgggagcgggggctggagtacggccccgcgtaccaggccatcaagcacatcggccgcgggcgtggcgaggccctggcggagctgcacgtgccccaggccgtcgcggaggagctgagcgggtgccagctccaccccagcctcctggacgccgggttccagctcatcgacacggcgctggccgggtcgggcctcgtggaggagggagacgtctacctgcccgccgccctccgcggggtgcggttccaccgcaagccggcccccggacagacgctccatgcgcacctggggttgagcccggagaagccgcgcgaggcgtcggtgctggaggcgcggctggcgctcctggatgaccagggcgctcccgtcctcacggccgagcggttcatcatccgccgcgtcgagacccacgccgccgcctgggaggggctgccgggcgagtggttctaccgcctgcgctggcagccgctggagggctggcagccggaggccgccggggcgaggcgcgacgaggcctggctcctgttcgcggacgaccagggcgtggccgcggcggtggccgaccggctcgcgccctcgggcgtgaggaccacgctggtgcgcaaggcgcccggcgccgtcacgcgggcggggagcggctggacgctcgacccgagcacgccggagtccttccgcgagctgctcgccgcggtgccgccgggctccttcacccgcgtgctctacttctggggactggacgcgcccgcggtggaccccgagccggacgccctggacgcgcggctgcccgagacggaggccctcatctacctggtccaggcgctcgccgcccgggaggcgcagccggggccgcgcctgtggctcgtcaccgcgggcgccctccccggcgccgacggcgaggccgtgtcgcccatgcagaccccgctctgggccctgggccgcgtcctggactacgagcacccggagctgaagaccgtctcctgcgagctggagcgtcacgtcacggagaagcacgtcgccgtgctgacctcgctgctcctcgcgcccgaggtgcccgaccaggtgcgcgtgcgcgccgacggcccggccgcggcgcggctggcccgctacgcgcccccggccccccgggacacgccgcaccgcgtgtcgccgcacgaggcggccgtggtggtgtcgggcaacggccccgcgcccgcggagctgtccgtcgccaggcgacgggcccccgcggcccacgaggtcgaggtcctggtcacccacgcgcacctcgcctgggcgcccgcgtcccccgacgcgggcggcctcgtgccgggagggttcttcgcgggccgcgtcctccggctcggaggcgaggtccgcgggctctccgtgggccaggccatcgtggccccggcgggcggcgagctccgctcgattcgcacggtggacgccggtggcgtgcgcccgctgccggagcaggccgaccccgcgcgggcgctctgcgcgctggggacccgggccctggcgcggcacctcctgcgccagctcgcccccgtcgagcgaggccagacgctgctgctgctcggtggcgacctccccctcctccagtccctcgcgcgggtggccacgggccacggcgcgcgcgtcctcgccctggtccgggacggcgcgcacgcgcccgcgctccaggaggcgggcgccgaggtggtgggccggctggacgagcccggcgcgttcgagcggctgcgcgagcgcgccggtgcctcgggcgtcgacgtcctcatcaacctcgcgcccggcgccgcggtaccgcgggcgctcgacgtcttccgggcgggcggcgtcttcctggacctgacgcgcgcgtccccggtcggcccgaaggagctgcccgcctcggcgacgttcctctccctggggctcgcaccgctctacgcgcgcctggcgccgcccacgccggaagccatcgccgaggccagcgcgcccgcggaggcccctgcgcgcgtgtggagcctccaggagctgacgcagccgcgggcggaggcgccgctcgacggccaggcccacgtcctcggcgtgtcgtccgaacccgtgacggtgggggccgggagcgccccgctcgcgctctccgacaccgcgacgtacctcatcaccggcggcctcggcggcctcggcctccgggtggcgagcctgctggtggaccacggcgtgcgccacctggcgctcgtcggccgctcgccgccgagcgcgcaggcccggagcaccctccgtgagctggagcagcgcggcgtccgggtccaggtgttcgccgccgacgtcgcccggcgcgaggacctgctcggggtggtgaaggccctccaggccggcgacgccccgctccggggggtcatccactgcgccggcgtcctccaggacggcgcgctcctcggcctggaccgcgagcggttccgctcggtcctcggcagcaagctgagcggcgcgtggaacctgcacgccctcacccgggacctgccgctcgaccacttcgtgctcttctcctcggtgtcctcgatgctgggctcccccggccagggcaactacgccgcggcgaacgcgttcatggacggcctcgccgcctggcggcgcgcccgggggctgcccgcgctcagcgtcaactggggcgccttcagcgacatcggcctggcggccgtcggcgagcggcgcggagaccgcctggccgacaacggcatccccggcatccctcccgagaagggcatgaaggcgctcgagctggcgctgcgctggggcgagacgtccctgggcttcttccccttccccttcctcatctggcggaagctcaacccgcagattgcccgcaacacgctcttcaccgacatccgcgcggagagccgctaccgcgaggaggccagcggcgagcagcccatcgccgaggcgctcgccctcgcggacccggccgcccgccggagccacctcgacggcttcctgaagcagcagctctcgcgcgtactcaaggtgccggcggagcaggtccgcgacggcctcaccttcaaggagatgggcttcgactcgctgatggccatcgagctgcgcaaccggctggaggccggcctcacggtgtccctgtcgtcgacgctcgtctggcgctacccgacgctcgaggagatgtcgcggttcctgctccagctcgtcggcggcgggagcgagacgccctcggacgcacggccggcgtccgccgccccggcgcgccggggccgctggctggtgcgtgagaagccgagtccctccgcgaagctccggctgctctgcttcccgcacggcggcgccggcgcctcggccttccagggctggtggcagcagttcccggacacggtggaggtcctcaccttccagccgccggggcgcgaggagcggctccaggaggagcccgagcgcgacatgggggcgtatgcctctgccatcgtcgaggagctccagccgctgctcgacctgcccttcgccgtgttcggctacagcctcggcggcctcaccgggttcgccaccgtggccgagctgcggcggcgcaaccgcgtcaccccccggcacctgttcgtgtcgtccgccttcgccccgcacatccccttctccgagacgacgctggcccgggtctccacccagacctcggcgctgaaggccatggccttctaccagtccacgccggactcggtgttcaaggacccggagatgcaggcgctgctggcgcgctcgatggacgcggacaactccgtggtcgagtcctaccgcttcggcgcggagaagccgctggactgccccatcaccgccttcgcgggccggcaggacgacctcgctccgctccacgagcagctgcgctggaaggagctcaccacccaggccttcgagctgcggccgttcgagggcgaccacttcttcttcctgagagacaagcgcccggtgatggacgagctgcggcgccacctgctctccctcatcgcggactcccgggacggctcgtcccgctgaccccgcgcctcccccttcctgttcgaagcgagaacacacccatggatgcctcctcctcccgccccttcgagtcactggacgcccttcaggcgtgggtcgtcacctggctgtgcgagcatgcccggattccccagcaggccatcggcctcgacgtccgcctgagggacctcggcctcgactcggcccgggccacgaagctggtggccgcgctctccgagcgcttcggccggacgctcccgctgacgctgctgtgggactacccgaccgtcacggagctgtcccggcacctgatgaagcccgcgacggcggcggcggccggcgccacgccccgggccgagcgtcgcgcgagcggcgctccggaggaccgcgccatcgccgtcatcggcatggcctgccggttcccgggcggcgcggacacgcccgaggccttctgggagctgctggcgaaggggctcgatgtggtggggccggtgcccgcggaccgctacgacgcggaggccttctacgaccgtgacccgcgggcccgcggcaaggccgtcacccggtggggcggcttcctggagcggaccgacaccttcgacccctacttcttcggcatctctccgcgagaggccatcgagatggacccgcagcagcgcatgctgctggagctggcctgggaggcgttcgaggcggcgggcctgacgaaggacgggctccgggaccgcgcggtcggcatcttcgtcggcgccatgtggaatgactacgcccggctgtggcggggcggccccggcgccatcgagcagttcaccgcgaccggccaggacgacagcatcatctccgcccgcatctcctacgtcttcggcctgcgcggcccgagcctgacgctgaacaccgcctgctcgtcctcgctggtgtccgtgcacatggcctgccagagcctgctggcgggtgactccgagctggccctggcgggtggcgtcaacctcatccacggcccggacagcaccatcgccatgtcgaagttcggcggcatggcgccggacgggaagtgcaaggccttcgacgcgcgtggcaacggctacgtccgcggcgagggcgcgggcgtcgtgctgctcaagccgctgcaccgcgcgctcgaggacggcgacgtcatccacgcggtcatccgcggctcggccgtcaacaacgacggcttctccaacggcctgacctcgcccagcccggccgcccaggccgcgatgctgcgcgcggcctacgccagcgcgggcgtgtccccggcgcgggtccactacgtggaggcgcacggcaccggcaccatgctgggtgaccccatcgaggcgggcgcgctcggcgaggtgctcgggcagcaccccgagcgccagcagcccctgcgcatcgggtccgcgaagacgaacatcggccacctggaggccgccgccggcatggcgggcctcatcaagaccatcgaggcgatgaagcaccggcgcctgccaccctcgctccacttcgagcagcccaaccccaacatccacttcgaggggctccgcctgcgggtgcagaccgacctctcaccgtggcccaccgaggacgactcgctgctcgcgggcgtcagctccttcggcttcggcggcaccaacgcccacgtggtgctcgagtccttccagcccgcccggccggtggccatccccttctccgcgccggacgcggaggcgctgggcgcccggctcgtcgagctgcgggccttcgcggagcagcaccccgaggcctcgctggacgacctccagcgcgcgtgggtccagcgcctcaccgaggggccggtgcgccaggtgctcctcgcccgctcccggaaggacctcctggagcagctccgcgcgccggcacggccggcggctcccgcggggcgcggcgcgacgcggccggtcctcgtcttcaccggcaacggcgcgcagtaccccggcatggcgcgggacctgatgctcggggacgcgaccttccggcaggcggtcctccgctgtgacgaggcgctgcgggcgcacggctgcgacggggtggccctggcgctcgagtccggtgacttcgacgtgcggcgggtggacctggtgcagccgctgctgtgggcggtgcaggtcggcctcgtctccctgtggcgcgcgtggggtgtggagcccgccgccgtcgtcggccacagcgtcggcgaggtggccgccgcgtgggccgcgggcgcgctcgacctggacgaggccgcgcgcgtcgtggtccaccgcagccggctgcagcaggagtgcgcggaccagggcgggatggtgctcctcgagaccgacgccgagggcgcgcgctccctcgtgggcgccacggcggcggaggggctggccgtcgccgccctcaactcgcgcggcagcaccgtcgtgagcggccccacggcgagcctccaggcgctcatcacgaaggcggaggagcgcggggtgacggcgaggacggtccgcgtcaacgtcgcgtaccacggccccgggatggacgcgctgcgcgagcggctgcgggagtccctcgcggacctgcgtccgtgccctccccgcgtcccgatgtcctcctcggtgacggggggcgcggtggggggcgcgggcctggacgccggctactgggtccgcaacatggccgaccccgtgctgttcggtgacgcggtcgacgcgctggtgagggacggccaccgcctgttcctggaggtgggcccgcaccccacgctccaccgggccgtccaggagcagctccagcacggaggcgtggccgggggcttcgtcgcgacgagcctggagcgcgagcagcacggccgcgcccgcctgctggagaacctgggccacctgttcgagcgcggtgtccgcgtggcgtggcgggcgctgacgcggggaggcgcgccggcctccgcgggccaggcgcccggccccgtcctcctccccctctccgcccacacggacgtggcgctccagcagcgcgtggaggggctgcgcgcgtacctgcgggcgaacccggaggcggagctggaggccctggcccgtaccctgggccggggacgcacccacctgtcgcaccgctgcgcgctcgtggcctcctcggccggcgagctcgcggaggtcctggagcgcgcggccggcgccgacggcgcgcccgagctcatccgccccgcggcctccctcgacggccgcgcgaaggtggccttcgtgttccccgggcagggctcccagtggctcggcatgggccgccagctcctggagacggagcccgtcttccgcaaggcgctccatgagtgggaggccgtgctgggagagttcaccgactggtcgctgctggagcagctccacgccgacgccgcgacgtcccgcctgggcgaggtggacgtcatccagccgaccctcttcgccatccaggtcgcgctggcggcgctgtggcgctcgtggggcgtgcacccggacgccgtcatcggccacagcatgggcgaggtcgccgcggcctacgtcgcgggggcgctggacgcgcgtgacgcggcccgcatcatctgctcgcgaagccgcatcgcgaagaagacgagcggccgcggcagcatggccgtggtcgagctgtcccccgaggagtgccagcgcgccatcggcgcccgcgccgccacggtgagcctcgccgcctgcaacgggcccgcctcgtgcctgctgtcgggagagacggccgcggtggaggagattggcagggagctccaggcccggggcgtcttcttccggctcgtcaaggtggactacgcgtcccacagccctcagatggacgccatcaaggacgagctggcgcacgtccttcgcgagacgcacccgcgcccggccaccgtgccgttccactccaccgtggagaacgccctcctcgacgggacacggctggacgccggctactgggcgcgcaacctgcgcgagccggtgctcttcttccagggcatccaggcgctggaggcccgcggcttcaacgtgttcctggagctcagcccgcaccccgtcctcacctactccatctcgcagacggtgcgtccggagagcgcgctcatcctcacctcgacgaagcgcgaggaggacgagtcgcgggcgctgcgcacgtcgctgggcgcgctcttcgccgcgggcggcgccgtggactgggccgcgctgtacccgggcgtggcgacgcccgtcgagctgcccgcctatcccttccagcgggagcggttctggttcgacgaggcggaggtccagcggtccgcgcccgcgggcaccggccgccacgcgttcctcgagcgcttcctccgctcgccgtacccccaggagcgcctcatctgggagacggacctgagcctcgagcgcttcccgtacctcgtggaccatcgcgtcagcggcctcgccgtcttccccggcaccggctacatcgagtgcgtcctggaggcggcgaaggacctgtacggcgacgcacccgtcgagctgctcgaggtctccttcgagaaggccctcttcatcgaggacaccggcgcgaagacgctgcagctctgcatggagccggacggcgccggccaccgcttctccctgtcgagccacgacgcggcccagcagacgtggacccgccacgtgaatggccggctcgtgcccgccaccgccgagcaggccgcccacgcgcgccagcaggtccaggacgtgctggccggcgagccccacgcgccggtggacgtggggacgttctacgggaacctcgagcgcgcggggaacacctacggcccgaagttccaggccgtgaagtccctgcgctcgaacgccaggggcgtccgcggcgaggtcgtcatgggcgaggagctggccacccggggcacccgcatccaccccgtcgtcttcgacgcctgcctccaggtgatggccacgcccctctacatcgagaaggccgcggggggccgcgagagcgccttcatgccgacgcgggcggcgcgcatccgcttcttccgtgaggccgaccgggccgtggtcagccacgtgcgcttcctgaacggggccaccgccgacgcggccggcgacctcctcgcggacatcgacgtcacggacgggcagggccagctgctggcgcgggtggagcgcatctgcttccacctgctggagcacgccaccatggcgcagcgcccgtccaccgacatgctctaccggctgacgtgggaggaggctccctcgccggaggtcggcgcctcgctggcgcgccgctggctcgtcttcgcggacgaccagggcgtcgccgaggcggcggccgggctgctgtccgcctcggcgcatgccgtgcaccgcctccacccggcggacgccttccggcgcgacggcaacgacttccacatccgcccgaacgagcccgaggactaccgcgccgtgctccaggccctgctggcctcgcgcggccccgcgctgccgctcggcgtcctccacctgtggaacctccgcctgcccgagggcggcgccacggccgtgccggacctgcgggagatggcgaagctgggcaacctcagcctcacccacctgctgaaggcgctcgacggggccggcggctccgcgccgccgcgcgtctggattggcacgcggggcgcgtgggccctccccgggagcgagcaggggctggcgccggcccagtccgcggtctgggggctcacccgggccatcgcgcaggagcacccggccctgtggggcggcatcatcgacctggatccgctcgaggccccgcgcctccaggcggagcggctcgtcacggaggtctccgtcccccaaccggcggaggaccgcatcctctaccggcagggccggcgtcaggtgtcgcggctggtccgcgacacggcccccgtcagcgccgcgaccgccaccttccgcggcgaccgcggctacctcatcacagggggccttggtgagctgggcacgcgcctggcggcgtggatggtggcccgtggcgcgcgccacctcatcctggcggggcgcaaggcgctgccggcccggcggacgtggaagcaggacgggcacgacgcggcgacccgggcgaggattgacgccgtcctccacctggagcgcctgggcgctcacgtccacacgcccgcgctggacgtgggggacggcgcggcgctggccgccttcctcgccggctacgagcaggaggcgcggccccccatcgccggtgtcgcccacctcgcgggggtcatccggcccgagctgctggcccggctccgcgaggagtccctcgaggagtccttccagtcgaaggtctacggcgcgtggaacctgcaccaggccttcagcgggcgcgggctggacttcttcacgctgttctcatccgcctcggccgtcctgaactcgcccctgctggggagctacgccgcggccaacgccttcctggacggcctggcgcacttccgccgcgcgcggggcgagccggcccagtccctgaactggggcttctggagcgaggccggcatggccgcgcgggacctccacttcgacgcgagcgcgtccggccagcagggcatgaggagcttcagccccgcggtgggcctggacatcttcggcgggttgctgagccgcgacccggtgcagtcggtggtgatggacgtggactggaagctctggctcgaggcctacccggacgccgccgcgtcgccgctgctgcgcaccctgcgcgccagcgagggcgtggccgcgggctccgagcaggcggcgccggccgaggaggcctcgcggagcctgagggacctgccggcggaggagcgccggaaggccgtcgagggcatcctgctcaccgagctgtccgcggtcctcaagatgccggccgcgaagatttcccggacggcgctcttcgggagcatgggcctggactccatgctctcgctggagctgcgcaaccggctggagcggcgcacgggcctgacgctgccgggcaccctggcctggaactacccctccctcgagcggatggccgcgttcttcgtcgagaagctcggcggggaaggcgctggcgtggcgcggtccgagcgccagcaggccggcgccgcgagcgatggcgtggcgaaggagccggacatctcggcggaggtcatcgaggccctgatgcagctgggcgtcctcgaggcgaacgaccccgtgctccaggatccggtcgcgctcggcaacctggtgcgcgaggcccagcagtacctgaaggcgtgacgcggcgctgacggggggcgctcccggcggagcgcctcagggccgcgtcacgccctccgaggagaccggccaccgcgtcgtcgcccacaggtacagcagcagcgtcagcggctcgaagcgggcgtcggcggcctccgcgggcgcgacggtccgcgagaggctggccagccgggcgaggcgggtgaggtcggccagcgcggcccccagcgcgggaagctccagcaggaggtgggcgcgcagcgccagcgcgagcaccagcccgtcactcggcgcgggctccggctccgcgtccgcgtccgccagcagggcctccgcgagggacgcgacccagcgcgtccccttcggctgcggcccggccgggtcgagatagccctccagccgctcggcctcggacagcatgcgggcgcgcagctcgcccagccacccgggggtgagggccccgggagccacgcgcaggctctgggaggcgctgtccaggacgagggcgggctcccccagccctggcgcggacagccggtccgccagggcccgcacccactggaggtacatgccgcgcgcgagggtccgccgcccctccaccaccaccgagtccgggacgcgcgggacggacgtgagcccccgcagcgggtcctctggccagtccccgccggagccgcgggtcaccgtccgggccgcgcacgcgcactgcgggaggatgctgacctcgaagtggtctccacagtggatgacctgcagcgggaactggcggcaggtccacggcttcgcctccgcgcccccggccttgtggacctggcaggccccctgggcaaggaagggacagccatcgccctggaggatgggcgacaggacgtcgcgccggccgggattcacgggggcgaaggcgtcctcggcccgcccgggggccgccccgagcgccagccggcgcagtgtctccacggcgcgccggctctcctccaccgtcgtggggatgacgggatactgcgagcagcacgccccggagaggttgcagccgaccgacagctcgctggtggtgatgggcagccccggcgccccgggacgcccccgctcctcgggctgaagggcgtgctggaggaactggggtgtccggggcgcgaggtcccgctccgcgtcgtacggcgggagctcgggcggatgcagggggtcgtcgacctcgacgccggggccgaagggccgcagcagcaggaagcggcggagctggcctccgacccggcgatgcaggagctgtgtgtacgtcatgtagggtgaaacccccgagcaggagccccttggtaaccaccttcgcgggaggcgtccagaggctggccgccccccgggccgcgctccgggcggtgctcaggtgctcagggcccgcacgcgcgcggcgaggttctgcgtgaagagccggtagatgcgcagcgcggcggcctcgtgggtgtcgaggtagtgctcgaagtcggcgcggctgatgcgcagcgcgcgcacggaggtgcgcgcgcggacgtgcgcggagacgggggcgtcctgcaccagggagatttccccgaggtacgcgccgggccccagcgtgttgaggtgacgcgcgtcctgctcggggccgctgaagacctccacggtgccgtccagcaggacgaagaggcccgtgccccgcgcgcccttctccagcagcaccgtgcccggcgcgatgacgacctgccgcgcgagccggtagaggtccttcatgtcctccagggacagctcgccgaagatggggatggccttgaggaagcggtacccgttggcggcgggcgtcgccgcggccgtcgcgcgccgcagcagcacggcctcggcctccgcctccatgcccatgcgccgcagcagccgcgccttctccttcaccagcgcctcgtccgcccgcgcctcctcggagctgctcaccgcgtccgccagcaccgcgagcgcccggtgggtgaagccctccacgtccagcagcgtgcagacgcgcagcaccgcctccacgtaccgcgagtcctccggcttcaccccgcccagcgcctccacctcggcgtgcgcctggcccagctcgcggtagaggtccgccgcctcgaaggggcggcccaggcggaccaggcactgcgccatcgcctcgcgggcgcccaggcgccggtacacgtccagcgcgcgctccagcgctccggcccgctcgaaggccgcggcggcgcgctccgcctcgccagcgcgcaggtacgcctccgccgcctccaggtactgcccgctctggaagtagaggtccgccacggtggcgtcctcgccgtcgccctccatcaggcgggcggcgcccaggaagtcgcgcgcccgccgcagcacctccaccaggccccggcgctcccgggccggacgccgggccgcctcccggcgcaggcgctcgcgctgcgcgtctcccagttcctcgtagagctgcgccgcctgctccgtctcctggagctccaccagtgaccgcaccgcgcgcagcgccccctccggaggcgtgtcgatgcgcccgccccccgccccctggcctgacgttcctgtcatgtgctctccccttccggtcagaccgcccagcat